(19) United States
(12) Patent Application Publication
(10) Pub. No.: US 2009/0232907 A1
(43) Pub. Date: Sep. 17, 2009

(54) Compositions and methods for treatment of esophageal cancer

(75) Inventors: Eric Van Quaquebeke, St. Lambert (BE); Gentiane Simon, Frannes-lez-Buissenal (BE); Mohamed El Yazidi, Ganshoren (BE); Jerome Tuti, Jemeppe-sur-sambre (BE); Laurent Van Den Hove, Forest (BE); Francis Darro, Waterloo (BE); Robert Kiss, St-Pieters-Leeuw (BE)

Correspondence Address: Davidson, Davidson & Kappel, LLC, 485 7th Avenue, 14th Floor, New York, NY, 10018, US

(73) Assignee: Unibioscreen S.A., Brussels (BE)

(21) Appl. No.: 12/313,179

(22) Filed: Nov. 17, 2008

Related U.S. Application Data
(63) Continuation-in-part of application No. 12/227,090, filed on Nov. 5, 2008.
(60) Provisional application No. 60/746,560, filed on May 5, 2006.

(30) Foreign Application Priority Data
May 5, 2006 (GB) 0608900.7

(51) Int. Cl. A61K 33/24; A61K 31/4188; A61K 31/4166; A61K 31/473

(52) U.S. Cl. 424/649; 514/393; 514/398; 514/296

(57) ABSTRACT

Novel ureyl-substituted naphthalimide derivatives, pharmaceutically acceptable salts thereof and solvates thereof, are useful for making pharmaceutical compositions for the treatment of cell proliferative diseases such as cancer. The invention also provides methods of treating specific types of cancer such as prostate, esophageal, glioblastoma, gliosarcoma, NSCLC, head and neck, and breast with the compounds described herein alone and in combination with antineoplastic agents.

This application is a Continuation-In-Part application of U.S. patent application Ser. No. 12/227,090, filed Nov. 5, 2008, which is a U.S. national phase application of International application No. PCT/EP2007/003991, filed May 7, 2007, which claims priority to U.S. Provisional Application No. 60/746,560 filed on May 5, 2006 and GB 0608900.7, filed May 5, 2006; the disclosures of which are hereby incorporated by reference in their entireties.

### TECHNICAL FIELD

The present invention relates to novel substituted naphthalimide derivatives, methods for their production and their pharmaceutical uses as anti-tumor agents, in particular in the form of pharmaceutical compositions including them as active principles in the prevention and/or treatment of various forms of cancer.

### BACKGROUND OF THE INVENTION

Various kinds of substituted naphthalimides, including amonafide, are known in the art as having anti-tumour effect or other useful biological activity. In particular WO 2005/105753 discloses naphthalimide derivatives having a specific substitution pattern which are active in the treatment of cell proliferation diseases such as cancer.

Although the level of activity found for amonafide was and continues to be of high interest, this material does have significant deficiencies which indicate the continuing need for agents with improved properties. In the first place, amonafide was found to be too toxic for some patients: in particular it has produced substantial myelotoxicity leading to some deaths in patients receiving five daily doses of the drug. In addition, it was shown that amonafide has only moderate activity in leukemia models in mice. Also, it was shown that amonafide has no activity in human tumour xenografts in mice with colon, lung and mammary cancers. Thus, while amonafide shows significant biological activity, it does not have a substantially broad spectrum of activity in murine tumour models. Ajani et al. in Invest New Drugs (1988) 6:79-83 has shown that amonafide has poor activity when tested in primary human solid tumours in vitro.

Although the clinical activity of antiproliferative agents such as amonafide against certain forms of cancers can be shown, improvement in tumor response rates, duration of response, decrease of myelotoxicity and ultimately patient survival are still sought. There is also a need in the art for improving the efficacy of antiproliferative treatments in humans by providing suitable combinations of new drugs with conventional antineoplastic agents.

In view of the above-mentioned shortcomings of amonafide and the like, there is a need in the art for naphthalimide derivatives demonstrating a more promising activity/side effects balance.

### OBJECTS AND SUMMARY OF THE INVENTION

The present invention is based on the first unexpected finding that a naphthalimide derivative being substituted with a ureyl group at position 5 of the naphthalimidyl moiety is useful in the treatment of cell proliferation disorders, can be made in an efficient manner through a limited number of reaction steps, and does not exhibit some of the drawbacks of the previously known similar derivatives. The present invention is also based on the unexpected finding that such ureyl-substituted naphthalimide derivatives are easily accessible in good yield through hydrolysis of other known substituted naphthalimide derivatives. The present invention is also based on the unexpected finding that such ureyl-substituted naphthalimide derivatives exhibit a satisfactory chemical stability and can easily be formulated into medicaments, e.g. as a suspension in the form of nanoparticles or as a solution in the form of a salt.

It is an object of the present invention to provide a group of substituted naphthalimide derivatives represented by the structural formula (I)

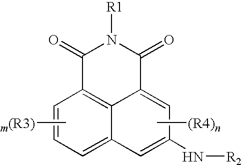

wherein:

R₁ is mono- or di-C₁₋₄ alkylamino-C₁₋₄ alkyl;

each of the substituents R₃ and R₄ is independently selected from the group consisting of hydrogen, halogen, C₁₋₄ alkyl, C₁₋₇ alkoxy, C₁₋₄ alkylthio, nitro, cyano, amino, protected amino and halo C₁₋₄ alkyl;

m is the number of substituents R₃ and ranges from 0 to 3;

n is the number of substituents R₄ and ranges from 0 to 2; and

R₂ is CONH₂;

and/or a pharmaceutically acceptable salt thereof and/or a solvate thereof and/or a metabolite thereof; for treatment of hyperproliferative diseases including cancer.

In accordance with the above group of compounds, the present invention is also directed to pharmaceutically acceptable compositions comprising one or more of the above compounds.

In accordance with the above group of compounds, the present invention is also directed to methods of treatment of hyperproliferative diseases including cancer comprising administering the pharmaceutically acceptable compositions comprising one or more of the above compounds.

In accordance with any of the above objects, the present invention is also directed to methods of treatment of cancers including esophageal cancer, gliomas, glioblastomas, gliosarcomas, NSCLC (non small cell lung cancer), head cancer, neck cancer, prostate cancer and breast cancer. In certain embodiments, the compounds of the present invention are administered as monotherapy. In still other embodiments, the compounds described herein are administered with conventional antineoplastic therapy. In still other embodiments, the compounds of the present invention combined with current antineoplastic therapy provide a synergistic effect.

In accordance with any of the above objects, the invention is also directed to methods of treatment utilizing the compounds recited herein that are well tolerated by the patient receiving treatment.

In accordance with any of the above objects, the methods of treatment of the present invention provide a significantly significant prolonged survival time.

In accordance with any of the above objects, the methods of treatment of the present invention provide a decreased tumor size, a stabilization in tumor size and/or a slowing of growth in tumor size.

In accordance with any of the above objects, the methods of treatment of the present invention provide a decrease in metastasis.

In accordance with any of the above objects, the invention is also directed to a method of treating esophageal cancer comprising administering to a patient in need thereof, N-{2-[2-(dimethylamino)ethyl]-1,3-dioxo-2,3-dihydro-1H-benzo[de]isoquinolin-5-yl}urea or a pharmaceutically acceptable salt thereof and/or a metabolite thereof in an amount effective to down-regulate one or more esophageal cancer cell pro-angiogenic chemokines.

In accordance with any of the above objects, the invention is also directed to a method wherein the chemokine is selected from the group consisting of CXCL-1, CXCL-2, CXCL-8 and combinations thereof.

In accordance with any of the above objects, the invention is also directed to a method comprising administering an antineoplastic agent.

In accordance with any of the above objects, the invention is also directed to a method wherein the antineoplastic agent is selected from the group consisting of taxol, temodal, dacarbazine, and pharmaceutically acceptable salts thereof and/or metabolites thereof.

In accordance with any of the above objects, the invention is also directed to a method wherein the antineoplastic agent is cisplatin.

In accordance with any of the above objects, the invention is also directed to a method wherein the esophageal tumor growth is slowed.

In accordance with any of the above objects, the invention is also directed to a method wherein the esophageal tumor growth is stopped.

In accordance with any of the above objects, the invention is also directed to a method wherein the esophageal tumor size is decreased.

In accordance with any of the above objects, the invention is also directed to a method wherein the patient experiences less hematotoxicity compared to treatment with a therapeutically equivalent amount of amonafide.

In accordance with any of the above objects, the invention is also directed to a method wherein the antineoplastic agent is pro-autophagic.

In accordance with any of the above objects, the invention is also directed to a method wherein the antineoplastic agent is pro-apoptotic.

In accordance with any of the above objects, the invention is also directed to a method wherein the effective amount is about at least about 10 mg/kg.

In accordance with any of the above objects, the invention is also directed to a method wherein one or more courses of treatment comprises administering one daily dose for at least about 5 consecutive days.

In accordance with any of the above objects, the invention is also directed to a method wherein one or more courses of treatment comprises administering one daily dose for at least about 3 times a week for a duration selected from the group consisting of (i) at least about 3 weeks, (ii) at least about 5 weeks and (iii) at least about 9 weeks.

In accordance with any of the above objects, the invention is also directed to a method wherein the dose of cisplatin is at least about 5 mg/kg.

In accordance with any of the above objects, the invention is also directed to a method wherein one or more courses of treatment comprises administering one daily dose per week for 5 weeks.

In accordance with any of the above objects, the invention is also directed to a method wherein the antineoplastic agent is temodal, or pharmaceutically acceptable salts thereof and/or metabolites thereof administered at a dose of at least about 80 mg/kg at least about 3 injections per week for about 3 weeks.

In accordance with any of the above objects, the invention is also directed to a method wherein the antineoplastic agent is temodal, or pharmaceutically acceptable salts thereof and/or metabolites thereof administered at a dose of 80 mg/kg at 3 injections per week for 9 weeks.

In accordance with any of the above objects, the invention is also directed to a method wherein the antineoplastic agent is dacarbazine, or pharmaceutically acceptable salts thereof and/or metabolites thereof administered at a dose of about 80 mg/kg at 3 injections per week for 3 weeks.

In accordance with any of the above objects, the invention is also directed to a method wherein the antineoplastic agent is dacarbazine, or pharmaceutically acceptable salts thereof and/or metabolites thereof administered at a dose of 80 mg/kg at 3 injections per week for 9 weeks.

In accordance with any of the above objects, the invention is also directed to a method wherein the antineoplastic agent is dacarbazine, or pharmaceutically acceptable salts thereof and/or metabolites thereof administered at a dose of about 10 mg/kg at 3 injections per week for 3 weeks.

In accordance with any of the above objects, the invention is also directed to a method wherein the antineoplastic agent is dacarbazine, or pharmaceutically acceptable salts thereof and/or metabolites thereof administered at a dose of 10 mg/kg at 3 injections per week for 9 weeks.

In accordance with any of the above objects, the invention is also directed to a method comprising administering to a patient in need thereof, a substituted naphthalimide derivative represented by the structural formula (I)

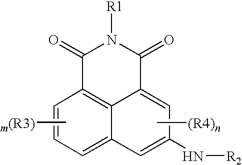

wherein:

R₁ is mono- or diC₁₋₄ alkylamino-C₁₋₄ alkyl;

each of R₃ and R₄ is independently selected from the group consisting of hydrogen, halogen, C₁₋₄ alkyl, C₁₋₄ alkoxy, C₁₋₄ alkylthio, nitro, cyano, amino, protected amino and halo C₁₋₄ alkyl;

m is the number of substituents R₃ and ranges from 0 to 3;

n is the number of substituents R₄ and ranges from 0 to 2; and

R₂ is CONH₂

and/or a pharmaceutically acceptable salt thereof and/or a solvate thereof and/or a metabolite thereof in an amount effective to down-regulate one or more esophageal cancer cell pro-angiogenic chemokines.

In accordance with any of the above objects, the invention is also directed to a pharmaceutical composition for injection comprising a therapeutically effective amount of N-{2-[2-(dimethylamino)ethyl]-1,3-dioxo-2,3-dihydro-1H-benzo[de]isoquinolin-5-yl}urea for the treatment of esophageal cancer in a pharmaceutically acceptable carrier comprising a liquid comprising an amount of lactic acid suitable for parenteral administration.

### DEFINITIONS

As used herein with respect to a substituting group, and unless otherwise stated, the term “alkyl” means straight and branched chain saturated acyclic hydrocarbon monovalent radicals having from 1 to 4 carbon atoms such as, for example, methyl, ethyl, propyl, n-butyl, 1-methylethyl (isopropyl), 2-methylpropyl (isobutyl), and 1,1-dimethylethyl (ter-butyl).

As used herein with respect to a member of a substituting group, and unless otherwise stated, the term “alkylene” means a divalent hydrocarbon radical corresponding to the above defined alkyl such as, but not limited to, methylene, bis(methylene), tris(methylene), tetramethylene, and the like.

As used herein with respect to a substituting group, and unless otherwise stated, the terms “alkoxy” and “alkylthio” refer to substituents wherein an alkyl group such as defined hereinabove is attached to an oxygen atom or a divalent sulfur atom through a single bond, such as but not limited to methoxy, ethoxy, propoxy, butoxy, isopropoxy, sec-butoxy, tert-butoxy, thiomethyl, thioethyl, thiopropyl, thiobutyl, and the like.

As used herein with respect to a substituting atom, and unless otherwise stated, the term “halogen” means any atom selected from the group consisting of fluorine, chlorine, bromine and iodine.

As used herein with respect to a substituting group, and unless otherwise stated, the term “haloalkyl” refers to an alkyl radical (such as above defined) in which one or more hydrogen atoms are independently replaced by one or more halogens (preferably fluorine, chlorine or bromine) such as, but not limited to, difluoromethyl, trifluoromethyl, trifluoroethyl, dichloromethyl and the like.

As used herein and unless otherwise stated, the term “solvate” includes any combination which may be formed by a ureyl-substituted naphthalimide (isoquinolinedione) derivative of this invention with a suitable inorganic solvent (e.g. hydrates formed from water) or a suitable organic solvent such as, but not limited to, alcohols, ketones, esters and the like.

As used herein and unless otherwise stated, the term “anti-migratory” refers to the ability of a pharmaceutical ingredient to stop the migration of cells away from the neoplastic tumor tissue and thus to reduce the colonization of new tissues by these cells.

The term “cell proliferative disorder” as used herein refers, but is not limited, to any type of cancer or other pathologic condition involving cell proliferation such as leukemia, lung cancer, colorectal cancer, central nervous system (CNS) cancer, melanoma, ovarian cancer, kidney cancer, prostate cancer, breast cancer, glioma, bladder cancer, bone cancer, sarcoma, head and neck cancer, liver cancer, testicular cancer, pancreatic cancer, stomach cancer, oesophaegal cancer, bone marrow cancer, duodenum cancer, eye cancer (retinoblastoma) and lymphoma.

### BRIEF DESCRIPTION OF THE DRAWINGS

FIG. 1 shows compound-induced hematotoxicity on platelets by a compound of this invention, as compared to amonafide.

FIG. 2 shows P-gp ATPase activity as measured by spectrophotometry for a compound of this invention, as compared to amonafide.

FIG. 3 shows (A) drug-induced pro-autophagic effects evaluated by quantification of acidic vesicular organelles (revealed as red fluorescent staining), and (B) drug-induced lysosomal membrane permeabilization (LMP) evaluated following acridine orange staining and quantification of green fluorescent staining, at different concentrations of a compound of this invention.

FIG. 4 shows senescence-associated β-galactosidase activity in DU-145 human prostate cancer cells induced by a compound of this invention, as compared to doxorubicin.

FIG. 5A shows UNBS3157 rapid hydrolysis to UNBS5162. FIG. 5B compares UNBS5162 oral bioavailability to i.v. bioavailability. FIG. 5C shows survival time of mice grafted with prostate cancer treated with UNBS5162, Taxol and both drugs together. FIG. 5D shows mean body weight of mice grafted with prostate cancer treated with UNBS5162, Taxol and both drugs together.

FIG. 6 shows quantitative determination of mRNA expression levels for CXCLs and CCL2 chemokines from human esophageal cancer cells.

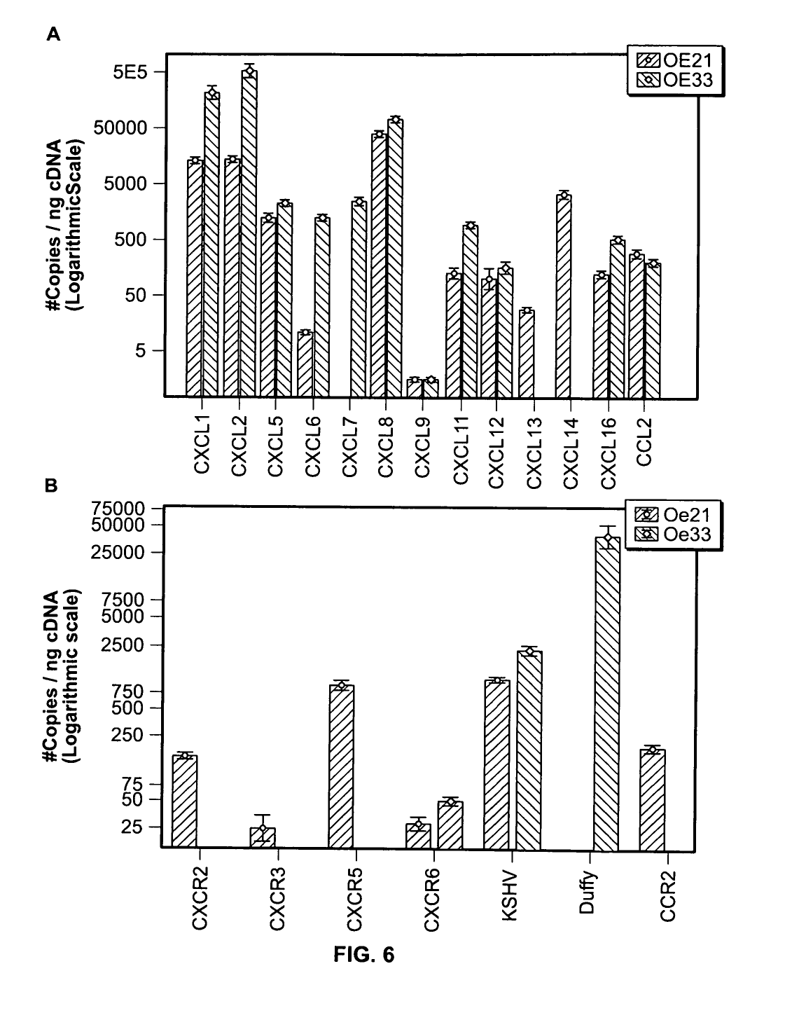

FIG. 7 shows CXCL-1 and CXCL-8 down-regulation induced by UNBS5162.

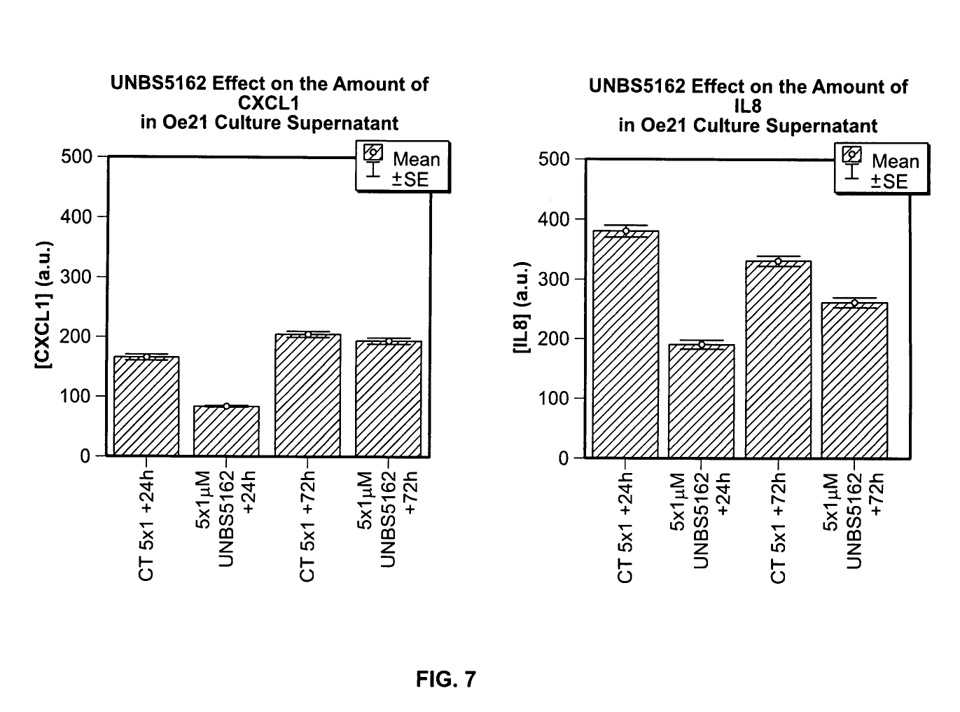

FIG. 8 shows anti-tumor effect of cisplatin, UNBS5162 and both drugs on mice grafted with esophageal cancer cells.

FIG. 9 shows UNBS5162-induced cell cycle arrest in G2 phase of glioblastoma cells.

FIG. 10 shows ELISA determination of CCL2 (A), CXCLI (B) and CXCL8 (IL-8; C) protein levels in untreated and UNBS5162-treated Hs683 cells at 1 μM either as a single treatment (grey bars) or as repeated (“chronic”; black bars) treatment (i.e. 1 μM each day for five days, “5×1”).

FIG. 11 shows Kaplan-Meier graph evidencing the anti-tumor effect of treatment comprising UNBS5162 administration after Temodal treatment.

FIG. 12 shows the Kaplan-Meier graph for the corresponding experiment. The marked prolongation of survival after treatment with combination 1 (UNBS5162 D14+Taxol D14) or combination 3 (Taxol D14+UNBS5162 D35) as evidenced by the TIC-values, was confirmed with the Kaplan-Meier (Log-rank statistics) analysis.

FIG. 13 shows mean body weight changes versus time for female B6D2F1 mice grafted subcutaneously with MXH-HI mammary tumor fragments left untreated (control) or treated i.v. (5i×3w) with UNBS5162 at a targeted dose level of 10 mg/kg and/or irradiated (1 irradiation) at a targeted dose level of 10 gray.

FIG. 14 shows mean tumor sizes evolution versus time for female B6D2F1 mice grafted subcutaneously with MXH-HI mammary tumor fragments left untreated (control) or treated i.v. (5i×3w) with UNBS5162 at a targeted dose level of 10 mg/kg and/or irradiated (1 irradiation) at a targeted dose level of 10 gray.

FIG. 15 shows the evolution of body weight in function of time and treatment with UNBS5162, radiation and both therapies combined.

### DETAILED DESCRIPTION OF THE INVENTION

In a first aspect, the present invention provides a group of substituted naphthalimide derivatives represented by the structural formula (I)

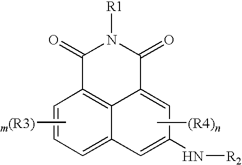

wherein:

R₁ is mono- or di-C₁₋₄ alkylamino-C₁₋₄ alkyl;

each of the substituents R₃ and R₄ is independently selected from the group consisting of hydrogen, halogen, C₁₋₄ alkyl, C₁₋₇ alkoxy, C₁₋₄ alkylthio, nitro, cyano, amino, protected amino and halo C₁₋₄ alkyl;

m is the number of substituents R₃ and ranges from 0 to 3;

n is the number of substituents R₄ and ranges from 0 to 2; and

R₂ is CONH₂;

and/or a pharmaceutically acceptable salt thereof and/or a solvate thereof and/or a metabolite thereof.

Metabolites of the derivatives represented by the structural formula (I) include, but are not limited to, the following:

mono-N-oxides and di-N-oxides thereof;

derivatives wherein R₂ is CONHOH; and

derivatives wherein R₃ and/or R₄ is hydroxyl.

Alternatively, mono- and di-N-oxides of the naphthalimide derivatives of this invention can be directly synthesized by treating a derivative represented by the structural formula (I) with an oxidizing agent such as, but not limited to, hydrogen peroxide (e.g. in the presence of acetic acid) or a peracid such as chloroperbenzoic acid.

The above defined novel compounds have in common the structural feature that the amino group of an amino-substituted naphthalimide (isoquinolinedione) such as, but not limited to, amonafide is substituted by an ureyl group or, in a metabolised form thereof, an ureyl N-oxide group.

In a preferred embodiment of this first aspect, the present invention relates to a sub-group of compounds wherein:

n=0 (when R₄ is not hydrogen), and/or

m=0 (when R₃ is not hydrogen), and/or

m=2, both substituents R₃ being adjacent and together with the carbon atoms to which they are attached forming a phenyl group, and/or

R₁ is an alkylene radical having from 1 to 3 carbon atoms and linked to a dimethylamino or diethylamino group, and/or

R₂ is CONH₂;

and/or a pharmaceutically acceptable salt thereof and/or a solvate thereof and/or a metabolite thereof.

In another preferred embodiment of this first aspect, the present invention relates to a sub-group of compounds wherein:

n=m=0 (when R₃ and R₄ are not hydrogen), and/or

R₁ is an alkylene radical having 1 or 2 carbon atoms and linked to a dimethylamino or diethylamino group, and/or

R₂ is CONH₂;

and/or a pharmaceutically acceptable salt thereof and/or a solvate thereof and/or a metabolite thereof.

In yet another preferred embodiment of this first aspect, the present invention relates to a sub-group of compounds, salts, solvates or metabolites thereof, wherein R₃ is not nitro when m equals 1. In yet another preferred embodiment of this first aspect, the present invention relates to a sub-group of compounds, salts, solvates or metabolites thereof, wherein R₃ and/or R₄ is selected from the group consisting of hydrogen, halogen, C₁₋₄ alkyl, C₁₋₇ alkoxy, C₁₋₄ alkylthio, cyano, amino, acylamino and halo C₁₋₄ alkyl.

In another preferred embodiment, the present invention relates to N-{2-[2-(dimethylamino)ethyl]-1,3-dioxo-2,3-dihydro-1H-benzo[de]isoquinolin-5-yl}urea, a salt or a metabolite thereof.

In a second aspect, the present invention provides a method for the production of ureyl-substituted naphthalimide (isoquinolinedione) derivatives represented by the structural formula (I) by hydrolysing a 5-substituted amonafide or amonafide derivative wherein the 5-substituent thereof is selected in such a way that it can be converted into an ureyl group through hydrolysis. Suitable 5-substituted amonafide derivatives for hydrolysis include, but are not limited to, compounds having the structural formula (II)

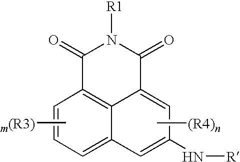

wherein:

each of m, n, R₁, R₃, and R₄ is as defined with respect to the structural formula (I), and

R′ is C₁₋₄ alkoxyamidocarbonyl or C₁ haloalkylamidocarbonyl.

Some compounds having the above structural formula (II) are already known e.g. from WO 2005/105753, but have been accessible only in very moderate yields, e.g. as a product of reacting amonafide with a C₁₋₄ alkoxycarbonyl isocyanate such as ethoxycarbonyl isocyanate or a C₁ haloalkylcarbonyl isocyanate such as trichloroacetyl isocyanate or trifluoroacetyl isocyanate. Therefore another aspect of the present invention is to design reaction conditions which permit to access these intermediates in better yields. A method for this purpose is one wherein said reaction of amonafide with a C₁₋₄ alkoxycarbonyl isocyanate or a C₁ haloalkylcarbonyl isocyanate is performed under conditions including:

the presence of a solvent, said solvent being selected from the group consisting of ethers (e.g. diethyl ether), ketones (e.g. 2-butanone or methylethylketone) and halogenated hydrocarbons (preferably having at most 2 carbon atoms and/or at least one chlorine atom, e.g. dichloromethane), and/or

a temperature below 0° C., e.g. a temperature ranging from about −30° C. to about −5° C., and/or

a molar excess of said C₁₋₄ alkoxycarbonyl isocyanate or C₁ haloalkylcarbonyl isocyanate, and/or quenching the reaction after its completion by adding water to the reaction mixture, thus avoiding (when a molar excess of C₁₋₄ alkoxycarbonyl isocyanate or C₁ haloalkylcarbonyl isocyanate is used) the formation of undesirable cyclisation by-products.

When one or more of the above reaction conditions are used, compounds having the above structural formula (II) can be obtained in significantly better yields, within the same or a shorter reaction time, than according to the prior art. The skilled person is capable of readily determining which combination of the afore-said process features, depending upon parameters such as the exact nature of R′, R₁ R₃ and R₄, is able to provide optimal yield within the shortest possible reaction time.

Hydrolysing a 5-substituted amonafide or amonafide derivative wherein the 5-substituent thereof can be converted into an ureyl group such as, but not limited to, compounds having the structural formula (II), may be performed either under acidic conditions or under basic conditions. The skilled person readily understands that this kind of hydrolysis is susceptible, depending upon parameters such as, but not limited to, pH, temperature, the kind of acid or base being used and the kind of solvent for the reaction mixture, to produce amonafide as a by-product which then has to be separated from the desired compound having the structural formula (I). The determination of optimal conditions for minimizing the formation of amonafide is within the general knowledge of the person skilled in the art. An advantage of the present invention is that it has proved quite easy to keep the proportion of residual amonafide in the final product below 3% by weight.

In a third aspect, the present invention provides a pharmaceutical composition comprising:

a therapeutically effective amount of an ureyl-substituted naphthalimide (isoquinolinedione) derivative represented by the structural formula (I), and/or a pharmaceutically acceptable salt thereof and/or a solvate thereof and/or a metabolite thereof; and

one or more pharmaceutically acceptable carriers.

In another aspect, the present invention provides combined preparations containing at least one ureyl-substituted naphthalimide (isoquinolinedione) derivative represented by the structural formula (I) and/or a pharmaceutically acceptable salt thereof and/or a solvate thereof and/or a metabolite thereof, and one or more antineoplastic drugs, preferably in the form of synergistic combinations as detailed below.

In another aspect, the invention relates to the unexpected finding that substituted naphthalimide (isoquinolinedione) derivatives represented by the general formula (I), and/or a pharmaceutically acceptable salt thereof and/or a solvate thereof and/or a metabolite thereof, have significantly higher biological activity, especially with respect to tumour cells, than amonafide while avoiding many of the above-mentioned drawbacks of amonafide. In particular, the ureyl-substituted naphthalimide derivatives according to the present invention have a significant anti-migratory effect. Migration refers to the biological process whereby cells migrate from a neoplastic tumor tissue and colonize new tissues, using blood or lymphatic vessels as major routes of migration, this biological process being also known as the metastatic process. Based on this finding, the present invention provides a method for treating and/or preventing tumours in humans. More specifically, the invention relates to a method of treatment of a host with a cellular proliferative disease, comprising contracting said host with an effective amount of an ureyl-substituted naphthalimide (isoquinolinedione) derivative represented by the structural formula (I), and/or a pharmaceutically acceptable salt thereof and/or a solvate thereof and/or a metabolite thereof.

In another embodiment, the invention provides the use of ureyl-substituted naphthalimide (isoquinolinedione) derivatives represented by the structural formula (I), and/or a pharmaceutically acceptable salt thereof and/or a solvate thereof and/or a metabolite thereof, as anti-tumour agents.

In another particular embodiment, the invention relates to a group of ureyl-substituted naphthalimide (isoquinolinedione) derivatives, as well as pharmaceutical compositions comprising them as an active principle, having the above structural formula (I) and being in the form of a pharmaceutically acceptable salt. The latter include any therapeutically active non-toxic salt which compounds having the structural formula (I) are able to form with a salt-forming agent. Such addition salts may conveniently be obtained by treating the ureyl-substituted naphthalimide (isoquinolinedione) derivatives of the invention with an appropriate salt-forming acid or base. For instance, ureyl-substituted naphthalimide (isoquinolinedione) derivatives having basic properties may be converted into the corresponding therapeutically active, non-toxic acid salt form by treating the free base form with a suitable amount of an appropriate acid following conventional procedures. Examples of such appropriate salt-forming acids include, for instance, inorganic acids resulting in forming salts such as but not limited to hydrohalides (e.g. hydrochloride and hydrobromide), sulfate, nitrate, phosphate, diphosphate, carbonate, bicarbonate, and the like; and organic monocarboxylic or dicarboxylic acids resulting in forming salts such as, for example, acetate, propanoate, hydroxyacetate, 2-hydroxypropanoate, 2-oxopropanoate, lactate, pyruvate, oxalate, malonate, succinate, maleate, fumarate, malate, tartrate, citrate, methanesulfonate, ethanesulfonate, benzoate, 2-hydroxy-benzoate, 4-amino-2-hydroxybenzoate, benzene-sulfonate, p-toluene-sulfonate, salicylate, p-aminosalicylate, pamoate, bitartrate, camphorsulfonate, edetate, 1,2-ethanedisulfonate, fumarate, glucoheptonate, gluconate, glutamate, hexylresorcinate, hydroxynaphtoate, hydroxyethanesulfonate, mandelate, methylsulfate, pantothenate, stearate, as well as salts derived from ethanedioic, propanedioic, butanedioic, (Z)-2-butenedioic, (E) -2-butenedioic, 2-hydroxybutanedioic, 2,3-dihydroxybutane-dioic, 2-hydroxy-1,2,3-propane-tricarboxylic, cyclohexane-sulfamic acid and the like.

Ureyl-substituted naphthalimide (isoquinolinedione) derivatives having the structural formula (I) having acidic properties may be converted in a similar manner into the corresponding therapeutically active, non-toxic base salt form. Examples of appropriate salt-forming bases include, for instance, inorganic bases like metallic hydroxides such as, but not limited to, those of alkali and alkaline-earth metals like calcium, lithium, magnesium, potassium and sodium, or zinc, resulting in the corresponding metal salt; nitrogen-containing organic bases such as, but not limited to, ammonia, alkylamines, benzathine, hydrabamine, arginine, lysine, N,N′-dibenzyl-ethylenediamine, chloroprocaine, choline, diethanolamine, ethylenediamine, N-methylglucamine, procaine and the like.

Reaction conditions for treating the ureyl-substituted naphthalimide (isoquinolinedione) derivatives (I) of this invention with an appropriate salt-forming acid or base are similar to standard conditions involving the same acid or base but different organic compounds with basic or acidic properties, respectively. Preferably, in view of its use in a pharmaceutical composition or in the manufacture of medicament for treating cell proliferative diseases, the pharmaceutically acceptable salt will be designed, i.e. the salt-forming acid or base will be selected, so as to impart greater water-solubility, lower toxicity, greater stability and/or slower dissolution rate to the ureyl-substituted naphthalimide (isoquinolinedione) derivative of this invention.

The present invention further provides the use of an ureyl-substituted naphthalimide (isoquinolinedione) derivative represented by the structural formula (I), or a pharmaceutically acceptable salt or a solvate thereof and/or a metabolite thereof, as a biologically-active ingredient, i.e. an active principle, especially as a medicine or a diagnostic agent or for the manufacture of a medicament or a diagnostic kit. In particular the said medicament may be for the prevention or treatment of a pathologic condition selected from the group consisting of cell proliferative disorders.

The compounds according to this invention are highly active against several types of cancers. Therefore, due to their favorable pharmacological properties, the compounds according to this invention are particularly suitable for use as medicaments or in the preparation of medicaments and combined preparations for the treatment of patients suffering from diseases associated with cell proliferation, more especially for treating cancer.

Any of the uses mentioned above may also be restricted to a non-medical use (e.g. in a cosmetic composition), a non-therapeutic use, a non-diagnostic use, a non-human use (e.g. in a veterinary composition), or exclusively an in-vitro use, or a use with cells remote from an animal.

The invention further relates to a pharmaceutical composition comprising:

(a) one or more ureyl-substituted naphthalimide (isoquinolinedione) derivative represented by the structural formula (I), and/or a pharmaceutically acceptable salt thereof and/or a solvate thereof and/or a metabolite thereof, and
(b) one or more pharmaceutically acceptable carriers.

In another embodiment, this invention provides combined preparations, preferably synergistic combinations, of one or more ureyl-substituted naphthalimide (isoquinolinedione) derivatives represented by the structural formula (I), and/or a pharmaceutically acceptable salt thereof and/or a solvate thereof and/or a metabolite thereof, with one or more biologically-active drugs being preferably selected from the group consisting of antineoplastic drugs. As is conventional in the art, the evaluation of a synergistic effect in a drug combination may be made by analysing the quantification of the interactions between individual drugs, using the median effect principle described by Chou et al. in Adv. Enzyme Reg. (1984) 22:27. Briefly, this principle states that interactions (synergism, additivity, antagonism) between two drugs can be quantified using the combination index (hereinafter referred as CI) defined by the following equation:

CIₓ=EDₓ^{1c}/EDₓ^{1a}+EDₓ^{2c}/EDₓ^{2a}

wherein EDₓ is the dose of the first or respectively second drug used alone (1a, 2a), or in combination with the second or respectively first drug (1c, 2c), which is needed to produce a given effect. The said first and second drug have synergistic or additive or antagonistic effects depending upon CI<1, CI=1, or CI>1, respectively. As will be explained in more detail herein-below, this principle may be applied to a number of desirable effects such as, but not limited to, an activity against cell proliferation.

The invention further relates to a composition or combined preparation having synergistic effects against cell proliferation and containing:

(a) one or more antineoplastic drugs, and
(b) at least one ureyl-substituted naphthalimide (isoquinolinedione) derivative represented by the structural formula (I), and/or a pharmaceutically acceptable salt thereof and/or a solvate thereof and/or a metabolite thereof, and
(c) optionally one or more pharmaceutical excipients or pharmaceutically acceptable carriers, for simultaneous, separate or sequential use in the treatment or prevention of cell proliferative disorders.

Suitable antineoplastic drugs for inclusion into the synergistic antiproliferative pharmaceutical compositions or combined preparations of this invention are preferably selected from the group consisting of alkaloids, alkylating agents (including but not limited to alkyl sulfonates, aziridines, ethylenimines, methylmelamines, nitrogen mustards and nitrosoureas), antibiotics, antimetabolites (including but not limited to folic acid analogs, purine analogs and pyrimidine analogs), enzymes, interferon and platinum complexes. More specific examples include acivicin; aclarubicin; acodazole; acronine; adozelesin; aldesleukin; altretamine; ambomycin; ametantrone; aminoglutethimide; amsacrine; anastrozole; anthramycin; asparaginase; asperlin; azacitidine; azetepa; azotomycin; batimastat; benzodepa; bicalutamide; bisantrene; bisnafide; bizelesin; bleomycin; brequinar; bropirimine; busulfan; cactinomycin; calusterone; caracemide; carbetimer; carboplatin; carmustine; carubicin; carzelesin; cedefingol; chlorambucil; cirolemycin; cisplatin; cladribine; crisnatol; cyclophosphamide; cytarabine; dacarbazine; dactinomycin; daunorubicin; decitabine; dexormaplatin; dezaguanine; diaziquone; docetaxel; doxorubicin; droloxifene; dromostanolone; duazomycin; edatrexate; eflomithine; elsamitrucin; enloplatin; enpromate; epipropidine; epirubicin; erbulozole; esorubicin; estramustine; etanidazole; ethiodized oil I 131; etoposide; etoprine; fadrozole; fazarabine; fenretinide; floxuridine; fludarabine; fluorouracil; fluorocitabine; fosquidone; fostriecin; gemcitabine; Gold 198; hydroxyurea; idarubicin; ifosfamide; ilmofosine; interferon α-2a; interferon α-2b; interferon α-n1; interferon α-n3; interferon β-1a; interferon γ-1b; iproplatin; irinotecan; lanreotide; letrozole; leuprolide; liarozole; lometrexol; lomustine; losoxantrone; masoprocol; maytansine; mechlorethamine; megestrol; melengestrol; melphalan; menogaril; mercaptopurine; methotrexate; metoprine; meturedepa; mitindomide; mitocarcin; mitocromin; mitogillin; mitomalcin; mitomycin; mitosper; mitotane; mitoxantrone; mycophenolic acid; nocodazole; nogala-mycin; ormaplatin; oxisuran; paclitaxel; pegaspargase; peliomycin; pentamustine; peplomycin; perfosfamide; pipobroman; piposulfan; piroxantrone; plicamycin; plomestane; porfimer; porfiromycin; prednimustine; procarbazine; puromycin; pyrazofurin; riboprine; rogletimide; safingol; semustine; simtrazene; sparfosate; sparsomycin; spirogermanium; spiromustine; spiroplatin; streptonigrin; streptozocin; strontium 89 chloride; sulofenur; talisomycin; taxane; taxoid; tecogalan; tegafur; teloxantrone; temoporfin; teniposide; teroxirone; testolactone; thiamiprine; thioguanine; thiotepa; tiazofurin; tirapazamine; topotecan; toremifene; trestolone; triciribine; trimetrexate; triptorelin; tubulozole; uracil mustard; uredepa; vapreotide; verteporfin; vinblastine; vincristine; vindesine; vinepidine; vinglycinate; vinleurosine; vinorelbine; vinrosidine; vinzolidine; vorozole; zeniplatin; zinostatin; zorubicin; and their pharmaceutically acceptable salts.

Other suitable anti-neoplastic compounds for inclusion into the synergistic antiproliferative pharmaceutical compositions or combined preparations of this invention include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; anti-androgens such as, but not limited to, benorterone, cioteronel, cyproterone, delmadinone, oxendolone, topterone, zanoterone; anti-estrogens such as, but not limited to, clometherone; delmadinone; nafoxidine; nitromifene; raloxifene; tamoxifen; toremifene; trioxifene and their pharmaceutically acceptable salts; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin; azasetron; azatoxin; azatyrosine; baccatin III derivatives; balanol; batimastat; BCR/ABL antagonists; benzochlorins; benzoylstaurosporine; β-lactam derivatives; β-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-aminotriazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; clomifene and analogues thereof; clotrimazole; collismycin A and B; combretastatin and analogues thereof; conagenin; crambescidin 816; cryptophycin and derivatives thereof; curacin A; cyclopentanthraquinones; cycloplatam; cypemycin; cytarabine; cytolytic factor; cytostatin; dacliximab; dehydrodidemnin B; deslorelin; dexifosfamide; dexrazoxane; dexverapamil; didemnin B; didox; diethylnorspermine; dihydro-5-azacytidine; dihydrotaxol; dioxamycin; diphenyl spiromustine; docosanol; dolasetron; doxifluridine; droloxifene; dronabinol; duocarmycin SA; ebselen; ecomustine; edelfosine; edrecolomab; elemene; emitefur; epristeride; estrogen agonists and antagonists; exemestane; filgrastim; finasteride; flavopiridol; flezelastine; fluasterone; fluorodaunorunicin; forfenimex; formestane; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idoxifene; idramantone; ilomastat; imidazoacridones; imiquimod; immunostimulant peptides; insulin-like growth factor-1 receptor inhibitor; interferon agonists; iobenguane; iododoxorubicin; ipomeanol; irinotecan; iroplact; irsogladine; isobengazole; isohomohalicondrin B; itasetron; jasplakinolide; kahalalide F; lamellarin-N; leinamycin; lenograstim; lentinan; leptolstatin; leukemia inhibiting factor; leuprorelin; levamisole; liarozole; lissoclinamide; lobaplatin; lombricine; lonidamine; lovastatin; loxoribine; lurtotecan; lutetium texaphyrin; lysofylline; mannostatin A; marimastat; masoprocol; maspin; matrilysin inhibitors; matrix metalloproteinase inhibitors; merbarone; meterelin; methioninase; metoclopramide; MIF inhibitors; mifepristone; miltefosine; mirimostim; mitoguazone; mitolactol; mitonafide; mitotoxin fibroblast growth factor-saporin; mofarotene; molgramostim; human chorionic gonadotrophin monoclonal antibody; mopidamol; mycaperoxide B; myriaporone; N-acetyldinaline; N-substituted benzamides; nafarelin; nagrestip; naloxone; pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; octreotide; okicenone; onapristone; ondanestron; ondansetron; oracin; osaterone; oxaliplatin; oxaunomycin; palauamine; palmitoylrhizoxin; pamidronic acid; panaxytriol; panomifene; parabactin; pazelliptine; peldesine; pentosan; pentostatin; pentrozole; perflubron; perillyl alcohol; phenazinomycin; phenylacetate; phosphatase inhibitors; picibanil; pilocarpine; pirarubicin; piritrexim; placetin A and B; plasminogen activator inhibitor; propyl bis-acridone; prostaglandin J2; proteasome inhibitors; protein kinase C inhibitors; protein tyrosine phosphatase inhibitors; purine nucleoside phosphorylase inhibitors; purpurins; pyrazoloacridine; raltitrexed; ramosetron; ras farnesyl protein transferase inhibitors; ras inhibitors; ras-GAP inhibitors; retelliptine; rhenium 186 etidronate; rhizoxin; retinamide; rohitukine; romurtide; roquinimex; rubiginone B1; ruboxyl; saintopin; sarcophytol A; sargramostim; sizofuran; sobuzoxane; sodium borocaptate; sodium phenylacetate; solverol; somatomedin binding protein; sonermin; sparfosic acid; spicamycin D; splenopentin; spongistatin 1; squalamine; stem-cell division inhibitors; stipiamide; stromelysin inhibitors; sulfinosine; suradista; suramin; swainsonine; tallimustine; tamoxifen; tauromustine; tazarotene; tecogalan; tellurapyrylium; telomerase inhibitors; temozolomide; tetrachlorodecaoxide; tetrazomine; thaliblastine; thiocoraline; thrombopoietin; thymalfasin; thymopoietin receptor agonist; thymotrinan; thyroid stimulating hormone; tin ethyl etiopurpurin; titanocene; topsentin; tretinoin; triacetyluridine; tropisetron; turosteride; tyrosine kinase inhibitors; tyrphostins; ubenimex; urogenital sinus-derived growth inhibitory factor; urokinase receptor antagonists; variolin B; velaresol; veramine; verdins; verteporfin; vinxaltine; vitaxin; zanoterone; zilascorb; and their pharmaceutically acceptable salts.

Synergistic activity of the pharmaceutical compositions or combined preparations of this invention against cell proliferation may be readily determined by means of one or more tests such as, but not limited to, the measurement of the radioactivity resulting from the incorporation of ³H-thymidine in culture of tumour cell lines. For instance, different tumour cell lines may be selected in order to evaluate the anti-tumour effects of the tested compounds, such as but not limited to:

RPMI1788: human Peripheral Blood Leucocytes (PBL) Caucasian tumor line,

Jurkat: human acute T cell leukemia,

EL4: C57BI/6 mouse lymphoma, or

THP-1: human monocyte tumour line.

Depending on the selected tumour cell line, different culture media may be used, such as for example:

for RPMI1788 and THP-1: RPMI-1640+10% FCS+1% NEAA+1% sodium pyruvate+5×10⁻⁵ mercapto-ethanol+antibiotics (G-418 0.45 μg/ml);

for Jurkat and EL4: RPMI-1640+10% FCS+antibiotics (G-418 0.45 μg/ml).

In a specific embodiment of the synergy determination test, the tumour cell lines are harvested and a suspension of 0.27×10⁶ cells/ml in complete medium is prepared. The suspensions (150 μl) are added to a microtiter plate in triplicate. Either complete medium (controls) or the tested compounds at the test concentrations (50 μl) are added to the cell suspension in the microtiter plate. The cells are incubated at 37° C. under 5% CO₂ for about 16 hours. ³H-thymidine is added, and the cells incubated for another 8 hours. The cells are harvested and radioactivity is measured in counts per minute (CPM) in a β-counter. The ³H-thymidine cell content, and thus the measured radioactivity, is proportional to the proliferation of the cell lines. The synergistic effect is evaluated by the median effect analysis method as disclosed herein-before.

The pharmaceutical composition or combined preparation with synergistic activity against cell proliferation according to this invention may contain the ureyl-substituted naphthalimide (isoquinolinedione) derivative having the structural formula (I), and/or a pharmaceutically acceptable salt thereof and/or a solvate thereof and/or a metabolite thereof, over a broad content range depending upon the precise contemplated use and the expected effect of the preparation. Generally, the ureyl-substituted naphthalimide (isoquinolinedione) derivative content of the combined preparation is within the range of about 0.1 to about 99.9% by weight, preferably from 1 to 99% by weight, more preferably from 5 to 95% by weight.

The pharmaceutical compositions and combined preparations according to this invention may be administered orally or in any other suitable fashion. Oral administration is preferred and the preparation may have the form of a tablet, aqueous dispersion, dispersable powder or granule, emulsion, hard or soft capsule, syrup, elixir or gel. The dosing forms may be prepared using any method known in the art for manufacturing these pharmaceutical compositions and may comprise as additives sweeteners, flavoring agents, coloring agents, preservatives and the like. Carrier materials and excipients are detailed hereinbelow and may include, inter alia, calcium carbonate, sodium carbonate, lactose, calcium phosphate or sodium phosphate; granulating and disintegrating agents, binding agents and the like. The pharmaceutical composition or combined preparation of this invention may be included in a gelatin capsule mixed with any inert solid diluent or carrier material, or has the form of a soft gelatin capsule, in which the ingredient is mixed with a water or oil medium. Aqueous dispersions may comprise the biologically active composition or combined preparation in combination with a suspending agent, dispersing agent or wetting agent. Oil dispersions may comprise suspending agents such as a vegetable oil. Rectal administration is also applicable, for instance in the form of suppositories or gels. Injection (e.g. intravenously, intramuscularly or intraperitoneally) is also applicable as a mode of administration, for instance in the form of injectable solutions or dispersions, depending upon the disorder to be treated and the condition of the patient.

Unless otherwise stated, the term “pharmaceutically acceptable carrier or excipient” as used herein in relation to pharmaceutical compositions and combined preparations means any material or substance with which the active principle(s), i.e the ureyl-substituted naphthalimide of this invention and optionally the antineoplastic drug, may be formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders.

Suitable pharmaceutical carriers for use in the said pharmaceutical compositions of the invention, and efficient ways for their formulation, are well known to those skilled in the art of pharmacology. There is no particular restriction to their selection within the present invention although, due to the usually low or very low water-solubility of the pteridine derivatives of this invention, special attention will be paid to the selection of suitable carrier combinations that can assist in properly formulating them in view of the expected time release profile. Suitable pharmaceutical carriers include additives such as wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, dissolving, spray-drying, coating and/or grinding the active ingredients, in a one-step or a multi-steps procedure, with the selected carrier material and, where appropriate, the other additives such as surface-active agents. The pharmaceutical compositions of the present invention may also be prepared by micronisation, for instance in view to obtain them in the form of microspheres usually having a diameter of about 1 to 10 μm, namely for the manufacture of microcapsules for controlled or sustained release of the biologically active ingredient(s). Suitable surface-active agents for use in the pharmaceutical compositions of the present invention are preferably non-ionic, cationic and/or anionic materials having good emulsifying, dispersing and/or wetting properties. Such suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable from coconut oil or tallow oil. Synthetic surfactants include sodium or calcium salts of polyacrylic acids; fatty sulphonates and sulphates; sulphonated benzimidazole derivatives and alkyl-arylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecyl-benzene sulphonic acid or dibutyl-naphtalenesulphonic acid or a naphthalene-sulphonic acid/formaldehyde condensation product. Also suitable for carrying out the invention are the corresponding phosphates, e.g. salts of phosphoric acid ester and an adduct of p-nonyl-phenol with ethylene and/or propylene oxide, or phospholipids. Suitable phospholipids for this purpose include, but are not limited to, the natural (originating from animal or plant cells) or synthetic phospholipids of the cephalin or lecithin type such as e.g. phosphatidyl-ethanolamine, phosphatidylserine, phosphatidylglycerine, lysolecithin, cardiolipin, dioctanyl-phosphatidylcholine, dipalmitoylphoshatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants for carrying out this invention include, but are not limited to, quaternary ammonium salts, preferably halides, having four hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2^{nd} ed. (Hanser Verlag, Vienna, 1981) and “Encyclopedia of Surfactants” (Chemical Publishing Co., New York, 1981).

Structure-forming, thickening or gel-forming agents may also be included into the pharmaceutical compositions and combined preparations of the invention. Suitable such agents include in particular, but are not limited to, highly dispersed silicic acid, such as the product commercially available under the trade name Aerosil; bentonites; tetraalkyl ammonium salts of montmorillonites (e.g., products commercially available under the trade name Bentone), wherein each of the said alkyl groups may contain from 1 to 20 carbon atoms; cetostearyl alcohol and modified castor oil products (e.g. the product commercially available under the trade name Antisettle).

Gelling agents which may also be included into the pharmaceutical compositions and combined preparations of the present invention include, but are not limited to, cellulose derivatives such as carboxymethylcellulose, cellulose acetate and the like; natural gums such as arabic gum, xanthum gum, tragacanth gum, guar gum and the like; gelatin; silicon dioxide; synthetic polymers such as carbomers, and mixtures thereof in any suitable proportions. Gelatin and modified celluloses represent a preferred class of gelling agents.

Other optional excipients which may also be present in the pharmaceutical compositions and combined preparations of the present invention include, but are not limited to, additives such as magnesium oxide; azo dyes; organic and inorganic pigments such as titanium dioxide; UV-absorbers; stabilizers; odor masking agents; viscosity enhancers; antioxidants such as, for example, ascorbyl palmitate, sodium bisulfite, sodium metabisulfite and the like, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid: flavoring agents such as natural vanillin; buffers such as citric acid and acetic acid; extenders or bulking agents such as silicates, diatomaceous earth, magnesium oxide or aluminum oxide; densification agents such as magnesium salts; and mixtures thereof.

Additional ingredients may be included in order to control the duration of action of the biologically-active ingredient in the compositions and combined preparations of the invention. Control release compositions may thus be achieved by selecting appropriate polymer carriers such as for example polyesters, polyamino-acids, polyvinyl-pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethyl-cellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition or combined preparation of the invention may also require protective coatings.

Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol, complexing agents such as cyclodextrins and the like, and mixtures thereof.

Since, in the case of combined preparations including the ureyl-substituted naphthalimide (isoquinolinedione) derivative having the structural formula (I), and/or a pharmaceutically acceptable salt thereof and/or a solvate thereof and/or a metabolite thereof, and an antineoplastic drug, both ingredients do not necessarily bring out their synergistic therapeutic effect directly at the same time in the patient to be treated, the said combined preparation may be in the form of a medical kit or package containing the two ingredients in separate but adjacent form. In the latter context, each ingredient may therefore be formulated in a way suitable for an administration route different from that of the other ingredient, e.g. one of them may be in the form of an oral or parenteral formulation whereas the other is in the form of an ampoule for intravenous injection or an aerosol.

The present invention further relates to a method for preventing or treating a cell proliferative disorder in a patient, preferably a mammal, more preferably a human being. The method of this invention consists of administering to the patient in need thereof an effective amount of an ureyl-substituted naphthalimide (isoquinolinedione) derivative having the structural formula (I), and/or a pharmaceutically acceptable salt thereof and/or a solvate thereof and/or a metabolite thereof, optionally together with an effective amount of an antineoplastic drug, or a pharmaceutical composition comprising the same, such as disclosed above in extensive details. The effective amount of the ureyl-substituted naphthalimide (isoquinolinedione) derivative is usually in the range of 0.01 mg to 20 mg, preferably 0.1 mg to 5 mg, per day per kg bodyweight for humans. Depending upon the pathologic condition to be treated and the patient's condition, the said effective amount may be divided into several sub-units per day or may be administered at more than one day intervals. The patient to be treated may be any warm-blooded animal, preferably a human being, suffering from said pathologic condition.

The following examples are intended to illustrate several embodiments of the present invention, including the preparation, pharmaceutical formulation and biological evaluation of the ureyl-substituted naphthalimides, without limiting its scope in any way.

### Example 1

### Preparation of ethyl({2-[2-(dimethylamino)ethyl]-1,3-dioxo-2,3-dihydro-1H-benzo[de]isoquinolin-5-yl}amino)carbonylcarbamate

1.086 g of amonafide were dissolved in 80 mL of 2-butanone at −20° C. under nitrogen atmosphere. Then 880 mg of ethoxycarbonyl isocyanate (2 molar equivalents) dissolved in 2 mL of 2-butanone were carefully added during 5 minutes by using a dropping funnel. Reaction temperature was maintained at −20° C. during 25 minutes under stirring. The reaction mixture was then warmed up to 45° C. during 40 minutes, after which time 250 μL of water was added. After this reaction quenching step, the precipitate formed was filtrated at 40° C. on paper. After drying, 1.1 62 g of the expected product (structural formula below) was obtained (yield: 76%). High performance liquid chromatography (hereinafter referred as HPLC) showed a purity above 95.6%. A slight amount of amonafide (about 2%) was still present.

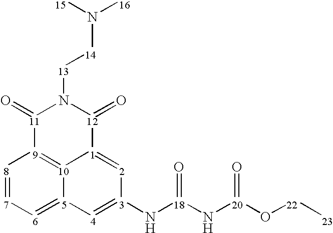

The desired product was characterized by:

proton nuclear magnetic resonance (300 MHz, CDCl₃), showing the same peaks as in example 4 of WO 2005/105753, and

electron-spray ionisation mass spectrum: showing a peak at M+H⁺=399; and the presence of an adduct at 2M+H⁺=797.

### Example 2

### Preparation of N-{2-[2-(dimethylamino)ethyl]-1,3-dioxo-2,3-dihydro-1H-benzo[de]isoquinolin-5-yl}urea

100 mg of the compound of example 1 were dissolved in 100 mL of NaOH 0.1 M. The reaction mixture was warmed up to reflux and maintained at this temperature during 1 hour. The mixture was analysed by HPLC, showing the presence of the expected urea (structural formula below) as the major product (yield 76%).

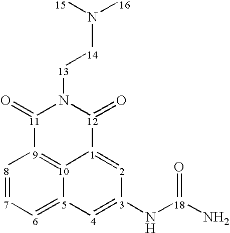

This product was characterised by the following techniques:

proton nuclear magnetic resonance (RMN ¹H, 300 MHz, DMSO) showing peaks at: 9.40 (NH-17, bs); 8.53 (H-2, d, J=1.8); 8.48 (H-4, d, J=1.8); 8.26-8.32 (H-6 and H-7, m); 6.18 (NH2-19, bs); 4.14 (H-14, t, J=6.6); 2.51 (H-13, m); and 2.21 (H-15 and H-16, s) ppm;

¹³C NMR (75.4 MHz, DMSO, TMS as internal standard) showing peaks at 37.5 (CH2, C-13); 49.9 (2×CH3, C-15 and C-16); 57.0 (CH, C14); 119.0 (CH, C-arom); 122.2 (C, C-arom); 122.9 (C, C-arom); 123.5 (C, C-arom); 123.9 (CH, C-arom); 127.8 (CH, C-arom); 128.6 (CH, C-arom); 132.7 (C, Carom); 133.8 (CH, C-arom); 140.3 (C, C-arom); 156.5 (C, C-18); and 163.8 (C, C-12); 164.0 (C, C-11) ppm; and

electron-spray ionisation mass spectrum: showing a peak at M+H⁺=327 and an adduct at 2M+H⁺=653.

### Example 3

### Effect on Overall Cell Growth

Tests were performed in order to rapidly, i.e. within 5 days, measure the effect of the compound of example 2 on the overall cell growth. The test measures the number of metabolically active living cells that are able to transform the yellow product 3-(4,5-dimethylthiazol-2-yl)-2,5-diphenyl tetrazolium bromide (herein referred as MTT) into the blue product formazan dye by mitochondrial reduction. The amount of formazan obtained at the end of the experiment, measured by means of a spectrophotometer, is directly proportional to the number of living cells. Optical density determination thus enables a quantitative measurement of the effect of the investigated compounds as compared to the control condition (untreated cells) and/or to other reference compounds.

Six human cancer cell lines described in table 1 were used in the following MTT tests. These cell lines cover six histological cancer types, being prostate, glioma, pancreas, colon, lung and breast cancers. Cells were allowed to grow in 96 well micro-wells with a flat bottom with an amount of 100 μl of cell suspension per well with 1,000 to 4,000 cells/well depending on the cell type used. Each cell line was seeded in a well known MEM 10% serum culture medium.

TABLE 1

| Cell line | ATCC code | tissue | literature reference |
|---|---|---|---|
| PC3 | CRL-1435 | Prostate | Invest. Urol. 17; 16-23, 1979; Cancer Res. |
| | | | 40: 524-534, 1980 |
| U-373MG | HTB-17 | Giloma | Acta Pathol. Microbial. Scand. 74: |
| | | | 465-486, 1968 |
| BxPC3 | CRL-1687 | Pancreas | Cancer Invest. 4: 15-23, 1986; Clin. Lap. |
| | | | Med. 2: 567-578, 1982 |
| LoVo | CCL-229 | Colon | Exp. Cell Res. 101: 414-416, 1976; J. Natl. |
| | | | Cancer Inst. 61: 75-83, 1978; Cancer Res. |
| | | | 39: 2630-2636, 1979 |
| A549 | CCL-185 | Lung | J. Natl. Cancer Inst. 51: 1417-1423, 1973; |
| | | | Int. J. Cancer 17: 62-70, 1976 |
| MCF-7 | HTB-22 | Breast | J. Natl. Cancer Inst. 51: 1409-1416, 1973 |

The detailed experimental procedure was as following: after a 24-hour period of incubation at 37° C., the culture medium was replaced by 100 μl of fresh medium in which the tested compound was previously dissolved, at the following molar concentrations: 10⁻⁹ M, 5·10⁻⁹ M, 10⁻⁸ M, 5·10⁻⁸ M, 10⁻⁷ M, 5·10⁻⁷ M, 10⁻⁶ M, 5·10⁻⁶ M and 10⁻⁵ M. Each experiment was repeated 6 times.

After 72 hours of incubation at 37° C. with (experimental conditions) or without (control condition) the compound to be tested, the medium was replaced by 100 μl MTT dissolved in RPMI (1640 without phenol red) at a concentration of 1 mg/ml. The micro-wells were subsequently incubated during 3 hours at 37° C. and centrifuged at 400 g during 10 minutes. MTT was removed and formazan crystals formed were dissolved in 100 μl DMSO. The micro-wells were shaken for 5 minutes and read on a spectrophotometer at wavelengths of 570 nm (maximum formazan absorbance) and 630 nm (back-ground noise).

For each experimental condition, the mean optical density was calculated, as well as the percentage of remaining living cells in comparison with the control.

Table 2 below shows the IC₅₀ values for the compound of example 2. IC₅₀ represents the range of molar concentrations at which the tested compound inhibited by 50% the overall tumor cells growth.

TABLE 2

| Cell line | IC₅₀(M) |
|---|---|
| PC3 | 10⁻⁵-5.10⁻⁶ |
| U-373MG | 10⁻⁵-5.10⁻⁶ |
| BxPC3 | 10⁻⁵-5.10⁻⁶ |

### Example 4

### Effect on Cell Migration

Cells of different cancer lines, i.e. U-373 MG (Glioma cancer) and A549 (Lung cancer) were seeded on culture flask 48 hours before the migration experiment. On the test day, cells were treated with or without the compound of example 2 in closed Falcon dishes containing a buffered medium at a controlled temperature (37.0±0.1° C.) for 12 or 22 hours, as noted in the right column of table 3. The compound of example 2 was used at three non-cytotoxic concentrations (10⁻⁶ M, 10⁻⁷M, 10⁻⁸ M). Migration of the cells was observed by means of a CCD-camera mounted on a phase-contrast microscope. Statistical analysis of the migration, with the non-parametric Mann-Whitney test, was established for 25% and 50% of the most motile cells and for the entire cell population. Table 3 below shows the anti-migratory effect of the tested compound.

TABLE 3

| Cell line | Maximum effects | Conditions |
|---|---|---|
| U-373MG | −29% | 22 hours on the 25% of |
| | p < 0.001 | the most motile cells, |
| | | at 10⁻⁷ M |
| U-373MG | −24% | 22 hours on the 50% of |
| | p < 0.001 | the most motile cells, |
| | | at 10⁻⁷ M |
| U-373MG | −20% | 22 hours on the 100% of |
| | | cells, at the 10⁻⁷ M |

The data of table 3 demonstrate that the compound of example 2 induced a decrease in the migration level of U-373 MG cancer cells at the non-cytotoxic concentrations used in this study. In particular, this compound shows a statistically significant inhibition of cell migration.

### Example 5

### Nano-Particles Suspension Formulations

A nanoparticles suspension is used for the formulation of the compound of example 2. For this approach selected excipients (in particular tension-active agents) including polysorbate 80 (Tween 80), Texapon K12 (SDS), PVA (Polyvinyl alcohol), Lutrol F68 (Poloxamer 188), Lutrol F127 (Poloxamer 407), Hydroxypropyl-β-cyclodextrine, Sodium taurocholate and other phopholipids (Lipoid S PC-3 and Phopholipon 90H) are used.

After selecting the excipients and their amounts the suspension containing the compound of example 2 is prepared by simply adding the designed quantity thereof into the desired volume of water. The suspension is then submitted to turax at 24000 rpm at low temperature for preliminary particle size reduction. The suspension is then submitted to an emulsiflex homogenisator at high pressure. Three cycles of homogenisation at different pressures may be used to obtain the expected size particle, e.g. the first cycle is performed at 7000 psi during 7 minutes, the second cycle at 12000 psi during 8 minutes and finally the last cycle at about 21000-24000 psi during 30 minutes. A determination of the particle size distribution is then made by Lazer diffraction, 5 measures being made with 20 seconds between each measure. The average of these 5 measures represents the particle size distribution of the suspension.

### Example 6

### Preparation of 2,2,2-trichloro-N-[({2-[2-(dimethylamino)ethyl]-1,3-dioxo-2,3-dihydro-1H-benzo[de]isoquinolin-5-yl} amino)carbonyl]acetamide

A 3-necked 12 L round bottomed flask equipped with mechanical stirrer, reflux condenser, cooling ice bath, dropping funnel and temperature controller was charged under nitrogen with amonafide (90 g) and 3.6 L of methylethylketone (MEK). The resulting suspension was cooled to −10° C. and a solution of 120 g of trichloroacetylisocyanate (0.64 mole) in 600 mL MEK was added drop-wise over 35 min, while maintaining the temperature bellow -5° C. The reaction mixture was stirred between −10° C. and −5° C. for 3 h, followed by the slow addition of 2.8 mL of water. The mixture was allowed to warm at room temperature and the resulting solids isolated by filtration, washed on the filter with 100 mL of MEK, and air dried for two days to give 147 g of the desired compound (yield: 97%; purity: 98.1%). The characterizing spectra of this compound were the same as described in example 2 of WO 2005/105753.

### Example 7

### Alternative Preparation of N-{2-[2-(dimethylamino)ethyl]-1,3-dioxo-2,3-dihydro-1H-benzo[de]isoquinolin-5-yl}urea

A 3-necked 22-L round bottomed flask equipped with mechanical stirrer, reflux condenser, and temperature controller was charged with the compound of example 6 (250 g) and 7.5 L of a 5% solution of K₂CO₃ in water. The mixture was cooled to 10° C. with (ice bath), and then 7.5 L of methanol (MeOH) was added in one portion. The temperature rose to 20° C. The flask was removed from the ice bath and stirring was continued at ambient temperature until most of the starting material dissolved (about 30-45 min). The mixture was quickly filtered (clarified), to remove the small amounts of unreacted materials and other mechanical impurities. The mixture was stirred at room temperature for 2 hours, and 4 L of MeOH was charged in one portion. The mixture was heated at 54-56° C. for 3 hours. Reaction progress was monitored by HPLC to ensure completion. The reaction mixture was cooled (ice bath) and kept at 8-10° C. for 2 hours. The obtained solid was isolated by filtration, washed on the filter (2×100 mL of water), and then dried in air for 2 days to give 156 g (yield: 90%) of the desired compound (HPLC purity: 99.47%). The characterizing spectra of this compound were the same as described in example 2 herein-above.

### Example 8

### Lactic Acid Based Solution Formulation

A liquid solution of the compound produced according to example 2 or example 7 was obtained as follows.

First a 2% by volume Lactic Acid solution was made as follows: to a 50 mL volumetric flask, 40 mL of 0.9% NaCl solution for injection and, using a Class A-TD Pipette, 1.18 mL of lactic acid, 85% ACS reagent were added. The volume was then adjusted with 0.9% NaCl solution for injection to 50 mL and the whole was mixed by inversion.

To a 25 mL volumetric flask, 700 mg of the compound of examples 2 and 7 were weighed accurately. To this specified quantity, 10 mL of 0.9% NaCl solution for injection and 8.89 mL of the aforementioned aqueous 2% Lactic Acid solution were added. The solution obtained was stirred vigorously and sonicated for 10 minutes. The pH of the solution was between 6.4-6.6. The pH was then adjusted to 5.75 by careful addition of small portions (20 μL) of the aforementioned aqueous 2% Lactic Acid solution. 0.9% NaCl solution for injection was used to adjust to final volume of 25 mL. At that point dissolution of the compound of the invention was observed to be complete by visual examination and the solution was sterilized by passing the solution through a pre-sterilized syringe filter (e.g., Millipore filter-Durapore (PVDF), 0.22 μm), thus resulting into a 28 mg/mL solution.

From this stock 28 mg/mL solution, the diluted solutions presented in the following table were obtained in 10 mL volumetric flasks by following dilution steps with 0.9% NaCl for injection. In the table 4, the indicated dose corresponds to the assumption that the dosing volume for intravenous injection is 5 mL per kg.

TABLE 4

| | | | | | |
|---|---|---|---|---|---|
| Compound | 28 mg/mL | 24 mg/mL | 20 mg/mL | 16 mg/mL | 12 mg/mL |
| Conc. | | | | | |
| Dose | 140 mg/kg | 120 mg/kg | 100 mg/kg | 80 mg/kg | 60 mg/kg |
| Volume of | NA | 8.571 mL | 7.143 mL | 5.714 mL | 4.886 mL |
| 28 mg/mL | | | | | |
| stock | | | | | |
| solution | | | | | |
| Volume | NA | Adjust to | Adjust to | Adjust to | Adjust to |
| (adjusted to | | 10 mL | 10 mL | 10 mL | 10 mL |
| 10 mL) | | | | | |
| with 0.9% | | | | | |
| NaCI for | | | | | |
| injection | | | | | |
| Target | 8 mg/mL | 4 mg/mL | 2 mg/mL | 1 mg/mL | 0.5 |
| Conc. | | | | | mg/mL |
| Doses | 40 mg/kg | 20 mg/kg | 10 mg/kg | 5 mg/kg | 2.5 mg/kg |
| Volume of | 2.857 mL | 1.429 mL | 0.714 mL | 0.357 mL | 0.179 mL |
| 28 mg/mL | | | | | |
| stock | | | | | |
| solution | | | | | |
| Volume | Adjust to | Adjust to | Adjust to | Adjust to | Adjust to |
| (adjusted | 10 mL | 10 mL | 10 mL | 10 mL | 10 mL |
| to 10 mL) | | | | | |
| with 0.9% | | | | | |
| NaCI for | | | | | |
| injection | | | | | |

### Example 9

### Hematotoxicity of N-{2-[2-(dimethylamino)ethyl]-1,3-dioxo-2,3-dihydro-1-H-benzo[de]isoauinolin-5-yl}urea

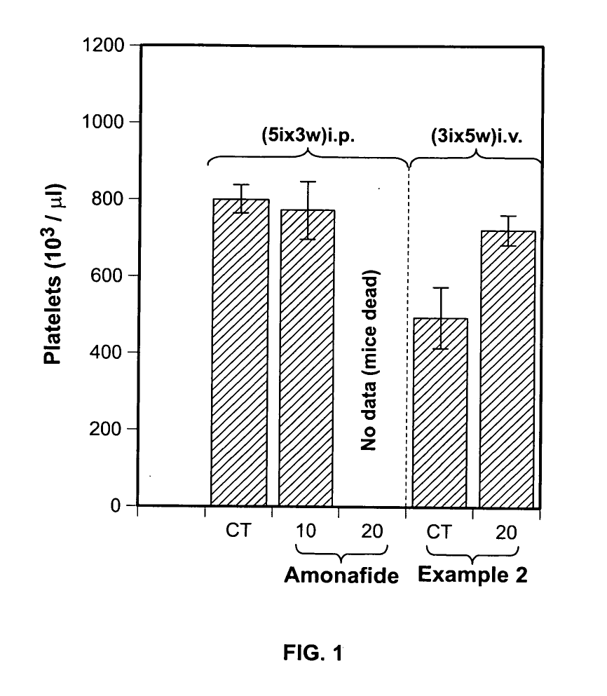

We have determined the compound-induced potential hematotoxicity of the compound produced according to example 2 or example 7 on platelets, red and white blood cells in comparison of the effect of amonafide on platelets, red and white blood cells. The effect of amonafide was evaluated at 10 mg/kg and 20 mg/kg, by the intra-peritoneal administration to mice. The administration schedule was five times a week for three consecutive weeks. The effect of the compound produced according to example 2 or example 7 was evaluated at 20 mg/kg, by the intra-venous administration to mice. The administration schedule was three times a week (on Mondays, Wednesdays and Fridays) for five consecutive weeks. The animals were sacrificed 3 days after the last injection. There were 10 mice per group. FIG. 1 illustrates results of this assay for compound-induced hematotoxicity on platelets. FIG. 1 shows that the mice tolerated 15 chronic administrations of amonafide at a dose of 10 mg/kg, while all animals died before receiving the complete set of 15 administrations of amonafide at a dose of 20 mg/kg. In contrast, FIG. 1 shows that the mice tolerated 15 chronic administrations of the compound produced according to example 2 or example 7 at a dose of 20 mg/kg. Thus, unlike amonafide, the compound produced according to example 2 or example 7 was found not to provoke hematotoxicity at therapeutic doses in these experimental conditions.

### Example 10

### Interaction of N-{2-[2-(dimethylamino)ethyl]-1,3-dioxo-2,3-dihydro-1H-benzo[de]isoquinolin-5-yl}urea with P-glycoprotein

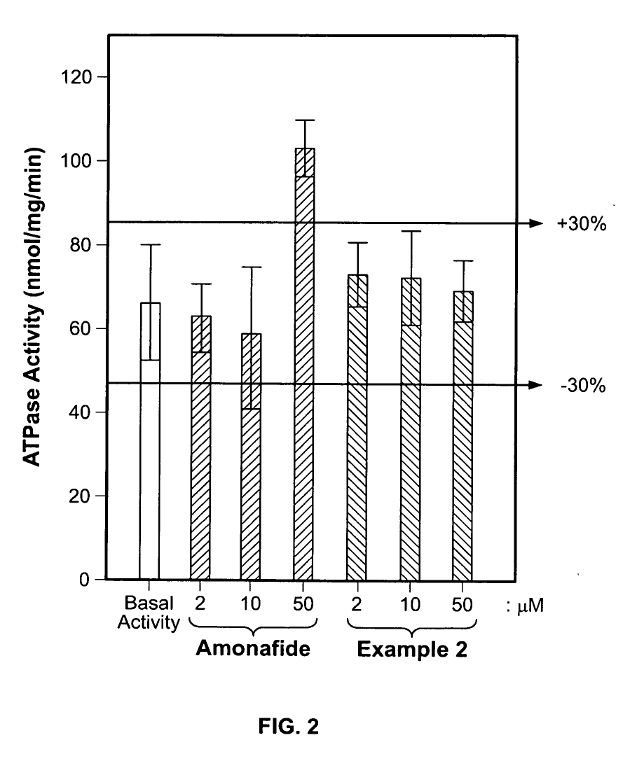

In order to test drug interaction with P-glucoprotein (herein-after referred as P-gp) we used an assay based on the study of modulation of ATPase activity from enriched P-gp membrane vesicle preparation (the following kit has been used: P-gp Drug interaction Assay Kit commercially available from SPI BIO France). P-gp ATPase activity was measured by a spectrophotometric method based on monitoring of ADP formation in the vesicle suspension medium. The basal ATPase activity was defined as the activity determined in the absence of any added drug. Modulation of basal activity was performed by adding amonafide or the compound produced according to example 2 or example 7 at different concentrations (2, 10, and 50 μM, respectively). The data shown in FIG. 2 indicate that, while amonafide when assayed at 50 μM significantly alter the ATPase activity, the compound of this invention does not affect ATPase activity, even.

### Example 11

### Inducing Effect of N-{2-[2-(dimethylamino)ethyl]-1,3-dioxo-2,3-dihydro-1H-benzo[de]isoquinolin-5-yl}urea on autophagy-related cell death in human cancer cells

A hallmark of topoisomerase P-targeting drugs is the induction of apoptosis; this is the consequence of an intracellular increase in the level of DNA damages by stabilization of the cleavable complex and/or a failure to achieve a complete chromosome segregation as a result of inhibition of topoisomerase II strand-passage activity. Amonafide is a topoisomerase II inhibitor and does induce apoptosis, a feature that we did not observe in human PC-3 (see Table 1) and DU-145 (ATCC Number: HTB-81) prostate cancer cells with the compound produced according to example 2 or example 7.

We used flow cytometry (according to the protacol found in Mijatovic et al. Neoplasia 2006) to determine the percentages of PC-3 and DU-145 positive cells for both Annexin V and propidium iodide and we observed that a maximum of 10% only of PC-3 or DU-145 cells underwent apoptotic processes following a treatment with 10 μM of the compound produced according to example 2 or example 7.

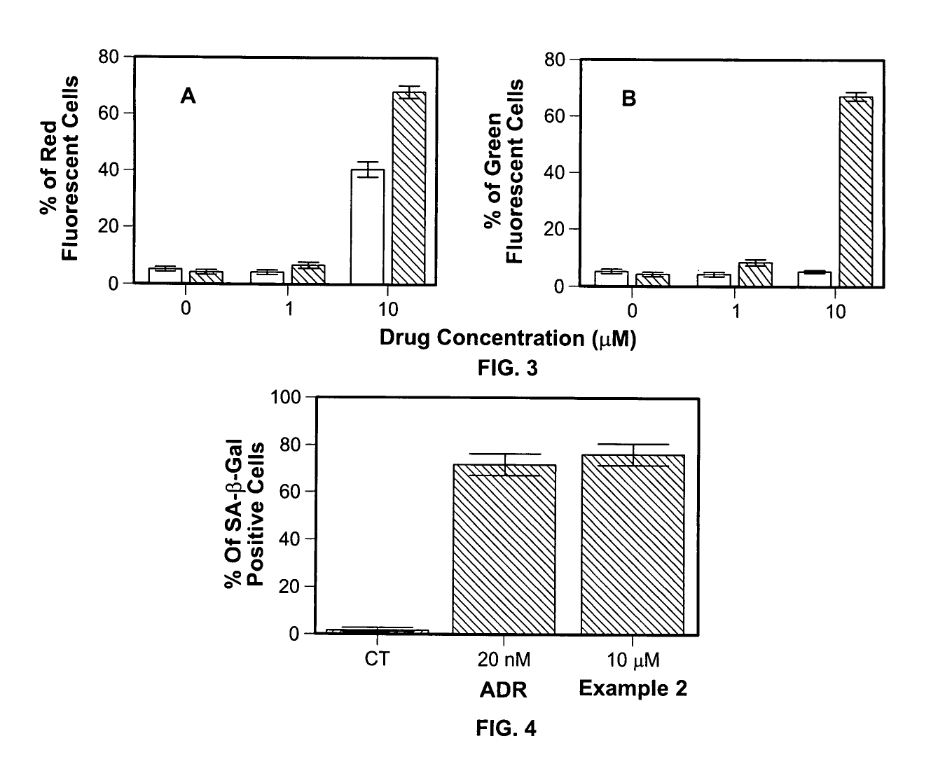

We observed pro-autophagic effects in PC-3 and in DU-145 cells treated with this compound. We quantified acidic vesicular organelles (revealed as red fluorescent staining) (according to the protocol found in Kanzawa et al. Cell Death Differ 2004), following acridine orange staining of PC-3 (gray bars) or DU-145 (black bars) cells after they have been treated with 0 (Control, untreated cells), 1 μM or 10 μM, and the consequent results are shown in FIG. 3A.

It is well known that lysosomes control cell death at several levels. In response to endogenous or exogenous stress (including chemotherapy), lysosomal membrane permeabilization (LMP) can occur, leading to the release of catabolic hydrolases that can mediate caspase-dependent apoptosis, caspase-independent apoptosis-like cell death or even necrosis following high levels of LMP. We thus quantified the “leakage” of acidic vesicular organelles (revealed as green fluorescent staining) (according to the protocol found in Nylandsted, J. et al. Heat Shock Protein 70 Promotes Cell Survival by Inhibiting Lysosomal Membrane Permeabilization, J Exp Med. (2004) 16; 200(4):425-35 and in Mijatovic et al., Neoplasia 2006) following treatment for 72 hours of PC-3 (gray bars) or DU-145 (black bars) cells after they have been treated with 0 (Control. untreated cells), 1 μM or 10 μM of the compound produced according to example 2 or example 7, and the consequent results are shown in FIG. 3B. We observed no LMP following treatment for 72 hours of PC-3 cells, while a marked drug-induced LMP process appeared in DU-145 cells when treated with 10 μM of a compound of this invention.

### Example 12

### Inducing Effect of N-{2-[2-(dimethylamino)ethyl]-1,3-dioxo-2,3-dihydro-1H-benzo[de]isoquinolin-5-yl}urea on Senescence in DU-145 Human Prostate Cancer Cells

This feature that the compound produced according to example 2 or example 7 induced non-apoptotic cell deaths has been furthermore observed at the morphological level by means of cellular imaging in human PC-3 and DU-145 prostate cancer cells treated with 10 μM of this compound for 6 days (cells seeded in Falcon flasks (25 cm²) and analysed for 6 days with quantitative video microscopy).

Senescence can be considered to be a type of “living cell death” because, although senescent cells maintain the integrity of their plasma membranes, they undergo permanent growth arrest and lose their clonogenicity. Senescence may act as a natural barrier to cancer progression.

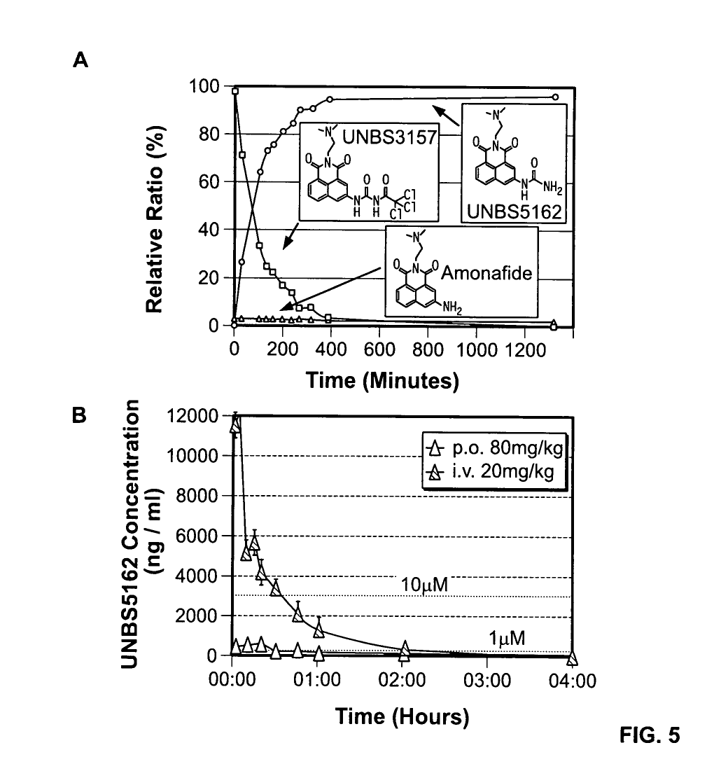

Features typical of senescence have been induced by the compound of example 2 in human DU-145 prostate cancer cells. A senescent cell is known to typically show morphological changes, such as a flattened cytoplasm and increased granularity. The induction of senescence-associated β-galactosidase activity is a specific event occurring in cells undergoing senescence, a feature that was observed once more in the current study (according to the protocol found in Dimri et al., A biomarker that identifies senescent human cells in culture and in aging skin in vivo, Proc Natl Acad Sci U S A. (1995) 92(20):9363-7), as evidenced in FIG. 4. Moderate doses (nM range) of doxorubicin (ADR) are known to induce senescence in wild-type human cancer cells. We therefore used doxorubicin as a positive control in our experiment. As shown in FIG. 4, 10 μM of the compound of this invention induced a similar percentage of senescence-associated β-galactosidase positive staining as 20 nM doxorubicin in DU-145 cells.

### Example 13

### Identification of Genes Targeted by N-{2-[2-(dimethylamino)ethyl]-1,3-dioxo-2,3-dihydro-1H-benzo[de]isoauinolin-5-yl}urea

At the biochemical level, senescence is accompanied by changes in metabolism, a feature that we also observed in the current study when performing genomic analyses on PC-3 cells treated in vitro with the compound of example 2.

At the genetic level, we also observed alterations to chromatin structure and gene-expression patterns in PC-3 cells when treating them with this compound.

We performed a first experiment of evaluation of gene targets by means of Affymetrix whole genome microarray using human PC-3 cancer cells grown in vitro and treated with the compound of the invention (N-{2-[2-(dimethylamino)ethyl]-1,3-dioxo-2,3-dihydro-1H-benzo[de]isoquinolin-5-yl}urea) either one time at 1 μM or 10 μM, or 5 times a week (one time a day during five consecutive days) at 1 μM (Full genome analyses were performed at the VIB MicroArray Facility (UZ Gasthuisberg, Catholic University of Leuven, Belgium) using Affymetrix Human Genome U133 set Plus 2.0 (High Wycombe, United Kingdom). The most salient data that we obtained are reported in table 5 and indicate that this compound, when assayed as a single high dose in vitro (acute in vitro treatment), markedly modified the nuclear organization and biogenesis by increasing significantly the levels of expression, at least at the mRNA level, of various types of histones. The second set of genes targeted by this compound belongs to a category of genes labeled “amino acid metabolism” (Table 5).

In the process of identifying senescence-associated genes in prostate cancer cells, the prior art teaches significant suppression of the ets homologous factor (EHF) in cancer cells in a state of DNA damage-induced senescence, and has shown that EHF provides substantial drug resistance in PC-3 prostate cancer cells by inhibiting senescence and cell cycle arrest. Interestingly, we found EHF to also be a target for the compound of the invention.

The E2F family of transcription factors is known to play an important role in cell cycle progression. E2F-1, in heterodimeric complex with another protein DP-1, is normally inactive because it is bound to hypophosphorylated pRb. When cells progress from the G1 to the S phase, pRb becomes hyperphosphorylated and releases the bound E2F-1/DP-1 heterodimer, which subsequently activates the transcription of genes involved in DNA such as TS and DHFR. The loss of functional pRb can give rise to increased free E2F-1 levels, and subsequently increased levels of TS and DHFR. As revealed by the genomic Affymetrix approach, we found that the treatment of PC-3 cells with 1 μM of the compound of example 2 one time a day during five consecutive days decreased by two times the mRNA levels of E2F-1.

During the initial phases of senescence, Rb might control the nucleation of heterochromatin at specific sites throughout the genome, which then spreads by the action of histone methyltransferases and recruitment of HP1 proteins. We have found that the compound of example 2 markedly increased the levels of heterochromatin in PC-3 cells through an increase of histones H1, H2 and H3, at least at the mRNA levels, in PC-3 cells (Table 5). In contrast, this compound decreased by 2.6 times the levels of mRNA expression of H2AFY.

TABLE 5
Gene Targets Affected by N-{2-[2-(dimethylamino)ethyl]-1,3-dioxo-2,3-dihydro-1H-
benzo[de]isoquinolin-5-yl}urea in vitro Treatment of PC-3
Human Prostate Cancer Cells

| | | | | | | | Boot | | Expression | Expression | CT |
|---|---|---|---|---|---|---|---|---|---|---|---|
| | Gene | List | | Population | Population | EASE | strap | | Level: | Level: | versus |
| System | Category | Hits | Total | Hits | Total | Score | Score | Genes | 10 μM | 5 × 1 μM | 1 μM |
| Biological | Nucleosome | 11 | 75 | 56 | 10401 | 5 × 10⁻¹² | 0.001 | H2AFY | 2.6 | NA | 1.7 |
| process | assembly | | | | | | | | | | |
| | | | | | | | | HIST1H3H | 0.3 | 0.6 | NA |
| | | | | | | | | HIST1H2BD | 0.3 | 0.3 | NA |
| | Chromatine | 11 | 75 | 85 | 10401 | 4 × 10⁻¹⁰ | 0.001 | HIST1H2AC | 0.4 | 0.2 | 1.0 |
| | assembly/ | | | | | | | | | | |
| | disassembly | | | | | | | | | | |
| | | | | | | | | HIST1H2BC | 0.4 | 0.7 | NA |
| | | | | | | | | HIST1H2BG | 0.4 | 0.5 | NA |
| | | | | | | | | HIST1H3D | 0.4 | 0.6 | 0.9 |
| | DNA | 11 | 75 | 160 | 10401 | 2 × 10⁻⁷ | 0.001 | HIST2H2AA | 0.4 | 0.5 | NA |
| | packaging | | | | | | | | | | |
| | | | | | | | | H2BFS | 0.5 | 0.5 | NA |
| | Nuclear | 11 | 75 | 180 | 10401 | 5 × 10⁻⁷ | 0.001 | HIST1H2BK | 0.5 | 0.3 | NA |
| | organization | | | | | | | | | | |
| | and | | | | | | | | | | |
| | biogenesis | | | | | | | | | | |
| | | | | | | | | HIST2H2BE | 0.5 | 0.3 | NA |
| KEGG | Amino | 8 | 15 | 236 | 1571 | 0.002 | 0.006 | ASNS | 3.6 | 0.9 | 1.3 |
| Pathway | acid | | | | | | | | | | |
| | metabolism | | | | | | | | | | |
| | | | | | | | | SARS | 2.6 | 0.9 | 1.0 |
| | | | | | | | | PHGDH | 2.4 | 1.0 | 1.4 |
| | | | | | | | | ALDH1A3 | 2.3 | 3.7 | 0.7 |
| | | | | | | | | CBS | 2.2 | 1.0 | 1.0 |
| | | | | | | | | BCAT1 | 2.1 | 0.8 | NA |
| | | | | | | | | CTH | 2.0 | 0.9 | 1.1 |
| | | | | | | | | SAT | 0.5 | 0.8 | 1.1 |

^{a}NA: Not available

In Examples 14-18 below, UNBS5162 has been studied with respect to specific tumor cells. The rational, methods, results and conclusions from the studies are discussed below.

### Example 14 N-{2-[2-(dimethylamino)ethyl]-1,3-dioxo-2,3-dihydro-1H-benzo[de]isoquinolin-5-yl}urea (UNBS5162) in Prostate Cancer

In Example 14, UNBS5162 was studied to evaluate the efficacy and the subsequent mode of action in prostate cancer based on applicants' publication Neoplasia. 2008 June; 10(6): 573-586, the disclosure of which is hereby incorporated by reference in its entirety. The effects mediated by UNBS5162 in vitro and in vivo in the models of prostate cancer were analyzed. The data are presented below.

Several naphthalimides have been evaluated clinically as potential anticancer agents. The UNBS3157, a naphthalimide that belongs to the same class as amonafide, was designed to avoid the specific activating metabolism that induces amonafide's hematotoxicity. The current study shows that UNBS3157 rapidly and irreversibly hydrolyzes to UNBS5162 without generating amonafide. In vivo UNBS5162 after repeat administration significantly increased survival in orthotopic human prostate cancer models. Results obtained by the National Cancer Institute (NCI) using UNBS3157 and UNBS5162 against the NCI 60 cell line panel did not show a correlation with any other compound present in the NCI database, including amonafide, thereby suggesting a unique mechanism of action for these two novel naphthalimides. Affymetrix genome-wide microarray analysis and enzyme-linked immunosorbent assay revealed that in vitro exposure of PC-3 cells to UNBS5162 (1 μM for 5 successive days) dramatically decreased the expression of the proangiogenic CXCL chemokines. Histopathology additionally revealed antiangiogenic properties in vivo for UNBS5162 in the orthotopic PC-3 model. In conclusion, the present study reveals UNBS5162 to be a pan-antagonist of CXCL chemokine expression, with the compound displaying antitumor effects in experimental models of human refractory prostate cancer when administered alone and found to enhance the activity of taxol when coadministered with the taxoid. The aim of the present study was to investigate the overall mechanism of action of UNBS5162 in the specific context of human prostate cancer, both in vitro and in vivo, given that recent research and clinical data continue to emphasize the potential of amonafide and its derivatives to combat prostate cancers.

### Materials and Methods

Compounds

UNBS3157, UNBS5181, UNBS5162, and amonafide were prepared at Unibioscreen as set forth above. Reference drugs were obtained as follows: taxol (Paclitaxel; S.A. Bristol-Myers Squibb, Brussels, Belgium), mitoxantrone (Sigma, Bornem, Belgium), doxorubicin (Adriamycin; Pfizer Pharmacia, Puurs, Belgium), and temozolomide (TMZ; Schering Plough, Brussels, Belgium).

### Evaluation of In Vitro Cell Proliferation By Means of the MTT Colorimetric Assay

The overall growth of human cancer cell lines was determined by means of the calorimetric MTT (3-[4,5-dimethylthiazol-2-yl]-diphenyl tetrazolium bromide; Sigma) assay, as detailed previously.

### Flow Cytometry Analysis of Cell Cycle Kinetics

The cell cycle kinetics of prostate cancer cells left untreated or incubated with UNBS5162 were determined by flow cytometry analysis of propidium iodide (PI) nuclear staining, using previously detailed methodology. Each sample was evaluated in triplicate. Flow cytometry was undertaken using an Epics XL.MCL flow cytometer and the FACScan/CellQuest software system (Becton Dickinson, Miami, Fla.).

### Flow Cytometry Analysis for Apoptosis Determination

The determination of the percentage of cells undergoing apoptosis was performed using an Annexin V-FITC Apoptosis Detection Kit (Sigma) following the manufacturer's instructions. Each sample was evaluated in triplicate.

Flow Cytometry Analysis for Autophagy Determination

Autophagic effects of UNBS5162 were determined by quantifying acidic vesicular organelles (revealed as red fluorescence) after acridine orange (Sigma) staining of PC-3 or DU-145 cells. The cytoplasm and nucleus fluoresce green in acridine orange-stained cells, and the acidic compartments fluoresce red. The intensity of the red fluorescence is proportional to the degree of acidity and the volume of acidic vesicular organelles, including autophagic vacuoles. To quantify the development of acidic vesicular organelles, the cells were stained with acridine orange for 15 minutes and removed from the plate with trypsinization. Cells were then analyzed by flow cytometry. Each sample was evaluated in triplicate.

Cell Senescence Analysis

After the indicated treatments, cells were washed in PBS, fixed for 3 to 5 minutes (at room temperature) in 2% formaldehyde/0.2% glutaraldehyde, washed and incubated at 37° C. (in the absence of CO₂) with fresh senescence-associated β-Gal (SA-β-Gal) staining solution: 1 mg/ml of 5-bromo-4-chloro-3-indolyl P3-d-galactoside (X-Gal; Sigma). Staining was evident within 2 to 4 hours and maximal after 12 to 16 hours. To detect lysosomal β-Gal, the citric acid/sodium phosphate used was pH 4.0. As described in the study of Dimri et al. [Proc Natl Acad Sci USA. 1995; 92:9363-9367.], after fixing and staining with X-Gal, the number of cells positive for the SA-β-Gal activity (intense blue staining) was then counted independently by two different individuals (on 400 cells/plate). Representative photographs (original magnifications, x20) of stained cells from different experimental treatments were taken. As a positive control for SA-β-Gal expression, Adriamycin-treated cells were used.

Total RNA Extraction

Total RNA was extracted using the TRIzol isolation reagent (Life Technologies, Inc., Merelbeke, Belgium) according to the manufacturer's instructions. The RNA extracted was treated with DNase I (Life Technologies, Inc.) to eliminate any remaining genomic DNA. The quality and integrity of the extracted RNA were assessed using both the BioAnalyzer 2100 (Agilent, Toulouse, France) and gel electrophoresis.

Quantitative (Real-time) Reverse Transcription-Polymerase Chain Reaction (RT-PCR)

Reverse transcription reactions were carried out in a thermal cycler (Thermocycler; Westburg, Leusden, The Netherlands). The purification of the cDNAs produced was undertaken using the High Pure PCR Product Purification Kit (Roche Diagnostics, Mannheim, Germany) in accordance with the manufacturer's instructions. Quantitative PCR reactions were performed with 50 ng of purified cDNA in a LightCycler thermocycler instrument (Roche Diagnostics) using LCFastart DNA Master SYBR Green 1 (Roche Diagnostics). After amplification, data analysis was carried out by means of the “Fit points” algorithm of the LightCycler quantification software. A standard curve enabled cDNA quantification of samples to be effected. The primers used were provided by Invitrogen and selected using the HYBSIMULATOR software (Advanced Gene Computing Technology, Irvine, Calif.). The primers used were as follows: ets homologous factor (EHF): forward: 5′-GGTGTAATGAATCTCAACCC-3′; reverse: 5′-CGAACTCTTGGAAAGGGA-3′; E2F1: forward: 5′-AGGAAAAGGTGTGAAATCCC-3′; reverse: 5′-GGATGTGGTTCTTGGACTT-3′.

### Genomic Analysis

PC-3 prostate cancer cells were either left untreated (control) or treated with UNBS5162 at 1) 1, 2) 10, or 3) 1 μM once a day for 5 consecutive days (with a washout process and renewal of culture medium before each new addition of compound, i.e., “5×1” μM). Cells were scraped into cold PBS buffer (for RNA extraction) 72 hours after the last addition of UNBS5162 into the PC-3 culture medium. Full genome-wide analyses were performed at the VIB MicroArray Facility (UZ Gasthuisberg, Catholic University of Leuven, Leuven, Belgium) using the Affymetrix Human Genome U133 set Plus 2.0 (High Wycombe, UK).

Microarray Data Analysis

In addition to R, an open-source software environment for statistical computing, a set of functions called BioConductor was used for the analysis and comprehension of the genomic data. The quality controls in the Affymetrix microarray experiments were performed with the Simpleaffy package and agreed with Affymetrix guidelines. The background correction, expression quantification and normalization were performed using Robust Multichip Analysis. To select differentially expressed genes between two experimental conditions, probes for which no overlap occurred between intervals in the expression values obtained for each condition were first identified. The fold change between two experimental conditions was computed for each of these probes (without any value overlap) as the ratio between the two nearest unlog expression values observed for the two different conditions (i.e., the ratio closest to 1 between any two values from the two different conditions). Probes for which these ratios were above 2.0 or below 0.50 were then selected. The annotations of the genes finally selected in this way were retrieved from the Affymetrix Web site through the BioConductor package ghgu133plus2.h. The EASE software package downloaded from http://david.niaid.nih.gov/david/ease.htm was used to gather biologic information on the genes detected as over-expressed or downregulated by the microarray analysis. This software package was then used to rank functional clusters by means of the statistical overrepresentation of individual genes in specific categories relative to all the genes in the same category on the microarray.

Western Blot Analysis

Cell extracts were prepared by the lysis of subconfluent PC-3 cells directly in boiling lysis buffer (10 mM Tris, pH 7.4, 1 mM Na₃O₄V, 1% SDS, pH 7.4). Approximately 40 μg of extracted proteins (evaluated by the bicinchoninic acid protein assay; Pierce, Perbio Science, Erembodegem, Belgium) was then loaded onto a denaturing polyacrylamide gel. The proteins submitted to Western blot analyses were detected using primary antibodies provided by: 1) CST Technologies, Bioke, Leiden, The Netherlands: Rb (1:2000) and p-Rb (1:1000); 2) BD Transduction Laboratories, Erembodegem, Belgium: E2F1 (1:400); and 3) AbCam, Cambridge, UK:tubulin (1:3000, used as a loading control). The antibody for LC-3 protein (1:500) was kindly provided by Dr. Yasuko Kondo. Western blot analyses were performed as detailed previously.

Enzyme-Linked Immunosorbent Assays (ELISAs)

Two different Quantikine ELISA kits (R&D Systems, Abingdon, UK) for the quantification of human CXCL1 and CXCL8 (IL-8) were used in this study in accordance with the manufacturer's instructions. Cell culture supernatants were collected after different treatments and periods, with multiple aliquots taken and stored at −20° C. until analysis.

Assessment of Potential Hematotoxicity of UNBS5162

Investigation of the hematotoxic potential of UNBS5162 was undertaken by HemoGenix (Colorado Springs, Colo.). The effects of UNBS5162 on stem and progenitor cell populations from murine and human bone marrow were assessed using HALO (Hematopoietic/Hematotoxicity Assay through Luminescence Output) technology. Stem and hematopoietic progenitor cell populations were isolated from mouse and human bone marrow samples, plated at a concentration of 1.0×10⁴ cells/well and cultured for 5 and 7 days, respectively, in the presence of growth factors and increasing concentrations of UNBS5162 ranging from 0.5 nM to 50 μM. The proliferative or growth potential of cell populations was determined from intracellular ATP (iATP) concentrations and a luciferin/luciferase bioluminescence signal detection system. All samples were assayed in eight replicates. The results are presented as estimated inhibitory concentration (IC) values.

In Vivo Experiments Using Human Prostate Cancer PC-3 and DU-145 Xenografts

Orthotopic xenografts were obtained by injecting 2.5×10⁶ human PC-3 or DU-145 cells into the prostate of 6-week-old male nu/nu mice (n=9 animals per treatment group). All grafts were performed under anesthesia [saline/Rompun (Bayer, Leverkusen, Germany)/Imalgene (Merial, Lyons, France); 5:1:1 by volume]. The end point in these orthotopic experiments was the survival period of the tumor-bearing mice after the administration of UNBS3157, UNBS5162, or reference anticancer agents (taxol, mitoxantrone, and amonafide). However, for ethical reasons, animals were killed when 20% of body weight had been lost compared to that determined at the time of tumor grafting. All animals were weighed three times a week. Autopsies and histologic diagnoses were performed on each mouse to confirm the presence of tumor development; 100% was achieved. In the case of UNBS5162 experiments in the PC-3 model, after the sacrifice of animals, tumors were removed from both drug-treated [10 mg/kg, intravenous (i.v.)] and vehicle-treated mice, fixed in buffered formalin, embedded in paraffin, and 5-μm-thick sections taken. These histologic slides were then stained with hematoxylin and eosin for blood vessel counts.

All the in vivo experiments described in the current study were performed on the basis of authorization No. LA1230509 of the Animal Ethics Committee of the Belgian Federal Department of Health, Nutritional Safety, and the Environment.

In Vitro Characterization of UNBS3157

To assess the in vitro stability of UNBS3157, 4.7 mg of the compound (Batch No. 27) was added to a 100-ml volumetric flask containing 25 ml of a mixture of physiological saline/DMSO (95:5 v/v). The volume was adjusted to 100 ml with further saline/DMSO (95:5 v/v) to give a final compound of UNBS3157 of 10⁻⁴ M. The solution was placed in a thermostat-controlled water bath maintained at 37° C. One-milliliter aliquots of incubate were taken at times 0, 30, 105, 135, 160, 200, 240, 270, 320, 390, and 1320 minutes and were analyzed as described below; thereafter, the levels of UNBS3157, UNBS5162, and amonafide were determined. The kinetics of UNBS3157 degradation in vitro were determined by HPLC-UV (250 μm) analysis, using an Atlantis (Waters Corp, Milford, Mass.) dC18 5 μm, 4.6×150 mm analytical column, and a binary gradient system involving the following: mobile phase A, 0.1% aqueous formic acid; and mobile phase B, 0.05% formic acid in acetonitrile. The following gradient was applied at room temperature and pressure:

100% A/0% B to 80% A/20% B in 6 minutes

80% A/20% B to 0% A/100% B in 3 minutes

0% A/100% B to 100% A/0% B in 7 minutes.

UNBS3157, UNBS5162, and amonafide had retention times of 11.25, 6.05, and 5.76 minutes, respectively. The relative amounts of each compound were determined by comparison of peak areas assuming the same response coefficient for all compounds. The starting material UNBS3157 was determined to be 98.6% pure but contained 1.4% residual amonafide resulting from incomplete conversion to UNBS3157 during the synthetic process. The level of UNBS5162, if present in the starting material, was below the limit of detection of the method.

Determination of UNBS5162 Mouse Pharmacokinetics

Mouse in-life phase. Female B6D2F1 mouse (Charles River, L'Arbresle, France) were administered a single i.v. bolus injection of 20 mg/kg or a single oral dose of 80-mg/kg UNBS5162 as a solution (formulated in 0.5% lactic acid). Dosing volume was 10 ml/kg body weight. The i.v. injection was performed through the tail vein, and the oral dose was given by gavage. Blood sampling for pharmacokinetic analysis was performed by cardiac puncture after Nembutal intraperitoneal injection. The blood was collected over Li-heparin at 0.05, 0.08, 0.17, 0.25, 0.33, 0.5, 0.75, 1, 2, 4, 7, 16, and 24 hours after dose. Five animals were used per time point. Blood was kept on ice for a maximum of 2 hours before isolating plasma by centrifugation, and the resulting plasma was stored at approximately -70° C. until analysis. The samples were subsequently analyzed for UNBS5162 by liquid chromatography-mass spectrometry.

Bioanalytical Method.

Plasma concentrations of UNBS5162 were determined using liquid chromatography-mass spectrometry. The assay was shown to be linear, precise, and accurate within an analytical range from 10 to 1000 ng/ml (for the i.v. analytical batch) and from 5 to 500 ng/ml [for the per os (p.o., i.e. oral) analytical batch]. Briefly, solid phase extraction was performed using SPE Oasis HLB columns of 1 ml (Waters). UNBS5162 and the internal standard UNBS5181 were eluted using methanol, evaporated to dryness and reconstituted in starting solvent, a mixture (90:10 v/v) of 0.05% aqueous formic acid (mobile phase A) and 0.05% formic acid in acetonitrile (mobile phase B). Liquid chromatographic separation was effected using an Atlantis T3 column (50×2.1 mm; 3 μm), with an isocratic method with a 90:10 v/v ratio of mobile phases A/B at a flow rate of 250 μl/min for 12.5 minutes, followed by 2 minutes of 100% mobile phase B and then 4.5 minutes reconditioning with the starting solvent at a flow rate of 250 μl/min. Compound detection and quantification were performed by positive ion electrospray ionization on a QTOF Ultima mass spectrometer (Micromass, Manchester, UK).

Statistical Analyses

Data are expressed as means±SEM. Data obtained from independent groups were compared by the nonparametric Kruskall-Wallis (more than two groups) or Mann-Whitney U tests (two groups). The standard survival time analyses were carried out using the Kaplan-Meier curves and the log rank test. The statistical analysis was performed using Statistica software (Statsoft, Tulsa, Okla.).

### Results

### UNBS3157 Displays Antitumor Activity In Vivo in Orthotopic Human Prostate Cancer Models

The anticancer activity of UNBS3157 versus that of amonafide, mitoxantrone, and taxol, the latter two drugs approved for the treatment of hormone refractory prostate cancer, has been compared in the two orthotopic models of human hormone refractory prostate carcinoma developed in our group, namely PC-3 and DU-145.

In the PC-3 model, mitoxantrone failed to contribute any therapeutic benefit while revealing itself to be highly toxic at 2.5 mg/kg i.v. UNBS3157 displayed appreciable activity against PC-3 prostate carcinoma when administered orally at 160 mg/kg (% T/C: 151%, P=0.03; but not at the lower dose of 40 mg/kg (% T/C: 100%; data not shown). Amonafide (% T/C: 103% and 107%) at 40 mg/kg p.o. (higher doses were toxic; data not shown) was not active orally in this aggressive prostate cancer model.

In the DU-145 model, amonafide was inactive at 20 mg/kg i.v. and at lower doses, whereas UNBS3157 at 20 mg/kg i.v. (% T/C: 143%, P=0.01) and taxol also at 20 mg/kg i.v. (% T/C: 146%, P 0.007) contributed a therapeutic benefit. Furthermore, both amonafide (% T/C: 138%, P=0.04) and UNBS3157 (% T/C: 164%, P=0.03) contributed a significant therapeutic benefit when administered orally at 40 mg/kg but not at lower doses (data not shown). The therapeutic benefit contributed by taxol at 20 mg/kg i.v. did not significantly (P>0.05) differ from that contributed by UNBS3157 at 40 mg/kg P.O.

UNBS3157 is Hydrolyzed to UNBS5162 with No Generation of Amonafide

UNBS3157 is rapidly and extensively hydrolyzed in saline in vitro to UNBS5162 (90% in 270 minutes), without production of amonafide. Indeed, the level of amonafide remains constant at 1.4% during the 22-hour incubation period. UNBS5162 must therefore be considered the major in vitro hydrolysis product of UNBS3157. Of note, 5% DMSO (used for compound solubilization in different activity assays) did not increase the rate of hydrolysis.

UNBS5162 Displays Weak In Vitro Antiproliferative Activity

UNBS3157 and UNBS5162 display weak antiproliferative activity in vitro. Indeed, the mean antiproliferative activity IC₅₀ values determined against nine human cancer cell lines investigated (using the MTT calorimetric assay) were 19.8 and 17.9 μM for UNBS3157 and UNBS5162, respectively. IC₅₀ (μM) values for in vitro growth inhibition of human cancer cell lines by UNBS3157 and UNBS5162 in provided in Table 6 below.

TABLE 6
IC₅₀ (μM) Values for In Vitro Growth Inhibition of
Human Cancer Cell Lines By UNBS3157 and UNBS5162

| | Cell Lines | | | | | | | | |
|---|---|---|---|---|---|---|---|---|---|
| Compounds | PC-3 | DU-145 | U373-MG | Hs683 | HCT-15 | LoVo | MCF-7 | A549 | Bx-PC-3 |
| UNBS3157 | 15.1 | 23 | 3.9 | 9.9 | 29.3 | 26.4 | 44.3 | 8.7 | 17.2 |
| UNBS5162 | 17.3 | 16 | 4.7 | 8.5 | 28.8 | 8.9 | 46.5 | 21.2 | 9.1 |

IC₅₀ values correspond to concentrations that reduced by 50% the global growth of the tested cell lines after 3 days of culturing in presence of the compounds.

IC₅₀ values reported are the means calculated from six separate determinations. The SEM values are not reported for the sake of clarity. Additionally, it should be noted that the highest SEM value calculated was less than 5% of its mean value.

UNBS5162 Mouse Pharmacokinetics

The pharmacokinetic profiles of UNBS5162 in female mice after i.v. (20 mg/kg) and oral (80 mg/kg) administration were studied. Below limit of quantification values were included in the pharmacokinetic calculations as O, Systemic exposure after oral administration of 80 mg/kg was relatively low (Cₘₐₓ=510 ng/ml and AUC_{0-∞}=886 ng·h/ml) reflected in an absolute bioavailability calculated to be only 3.84%. The volume of distribution (V_{d}) and the total clearance were estimated to be 18.9 L/kg and 3.47 L/h per kilogram, respectively. The half-life after i.v. administration of 20-mg/kg UNBS5162 was estimated to be 3.8 hours. Post-i.v. UNBS5162 plasma levels of 10 μM are only maintained for approximately 30 minutes, whereas 1-μM levels are sustained for maximally 2 hours. Pharmacokinetic parameters in female mice after single i.v. and oral administration of UNBS5162 is provided in Table 7 below.

TABLE 7
Pharmacokinetic Parameters in Female Mice After Single
i.v. and Oral Administration of UNBS5162

| | Tₘₐₓ | Cₘₐₓ | AUC_{0-∞} | T_{1/2} | V_{d} | Cl (L/h per | F |
|---|---|---|---|---|---|---|---|
| Route/dose | (h) | (ng/ml) | (ng · h/ml) | (h) | (L/kg) | kilogram) | (%) |
| i.v./20 mg/kg | NA | 12,009 | 5767 | 3.8 | 18.9 | 3.47 | NA |
| p.o./80 mg/kg | 0.3 | 510 | 886 | NC | NA | NA | 3.84 |

AUC indicates area under the curve;
Cl, clearance from plasma;
Cₘₐₓ, maximum concentration;
F, absolute bioavailability;
NA, not applicable;
NC, not calculable;
T_{1/2}, half-life time;
Tₘₐₓ, time to maximum concentration;
V_{d}, volume of distribution.

UNBS5162 Increases the Therapeutic Benefits of Taxol In Vivo in the Orthotopic Human PC-3 Prostate Cancer Model

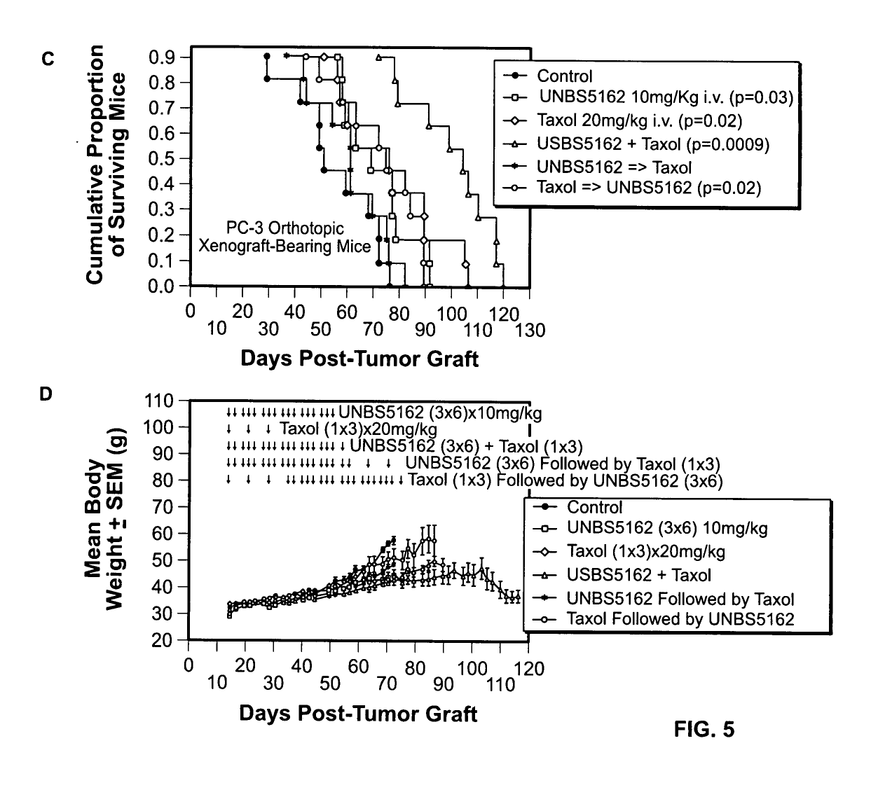

Given that UNBS3157 is rapidly hydrolyzed to UNBS5162 FIG. 5A and the latter is poorly systemically available after the oral dose FIG. 5B, open triangles), the anticancer activity of UNBS5162 was assessed by the i.v. route only. Although 1) UNBS5162 displays weak antiproliferative activity in vitro (see Table 7), 2) UNBS5162 plasma concentrations only range between ˜5.0 and 0.5 μM up to 2 hours after dose when administered i.v. at 20 mg/kg to mice (FIG. 5B), repeat i.v. administration of the compound at 10 mg/kg (three times a week for 6 consecutive weeks) contributed therapeutic benefits that were similar to repeat i.v. administration (once a week for 6 consecutive weeks) of 20-mg/kg taxol in the PC-3 orthotopic model (FIG. 5C). Preliminary data indicated that 10-mg/kg UNBS5162 was a dose as efficacious as 20 mg/kg. We thus decided to use the 10-mg/kg dose for chronic administration in xenograft studies in vivo, while keeping the 20-mg/kg dose in the pharmacokinetics study. Administering UNBS5162 before or after taxol did not modify the therapeutic benefit contributed by taxol alone. In sharp contrast, administering UNBS5162 at the same time as taxol to PC-3 orthotopic tumor-bearing mice significantly (P<0.01) increased the therapeutic benefit contributed by taxol alone (FIG. 5C). It is important to emphasize that compound treatment began not on engraftment but after the tumors had taken and showed considerable growth. Thus, the obtained data relate to decreases in tumor growth and metastatic processes in these orthotopic models. Combined treatment with taxol and UNBS5162 did not contribute higher toxicity (as monitored by mouse weight measurements three times a week during the duration of the experiment and through observation of mouse behavior) than single treatment with UNBS5162 or taxol alone (FIG. 5D).

Furthermore, in an evaluation of potential hematotoxicity, UNBS5162 at concentrations higher than 1 μM was toxic, as indicated by inhibited proliferation of murine and human hematopoietic stem and progenitor cells. The data are reported in Table 8 below.

TABLE 8
Hematoxicity Assay: Estimated IC Values

| | CFC-GEMM | BFU-E | GM-CFC | Mk-CFC |
|---|---|---|---|---|
| Mouse | | | | |
| IC₅₀ (μM) | 6.22 | 5.53 | 7.04 | 5.94 |
| IC₇₅ (μM) | 7.48 | 7.03 | 9.09 | 7.53 |
| IC₉₀ (μM) | 8.96 | 9.11 | 12.0 | 9.61 |
| Human | | | | |
| IC₅₀ (μM) | 2.57 | 3.6 | 3.74 | 4.05 |
| IC₇₅ (μM) | 5.67 | 8.16 | 8.12 | 9.22 |
| IC₉₀ (μM) | 12.8 | 17.6 | 19.9 | 20.1 |

BFU-E indicates burst-forming unit-erythroid; CFC-GEMM, colony-forming cell - granulocyte, erythroid, macrophage, megakaryocyte; GM-CFC, granulocyte-macrophage colony-forming cell; Mk-CFC, megakaryocyte colony-forming cell.

Characterization of UNBS5162 Mechanism of Action with Respect to Cell Proliferation and Cell Death

Use was made of computer-assisted phase-contrast microscopy (quantitative videomicroscopy) in the attempt to elucidate an overall picture of UNBS5162's mechanism of action. Six days of observation revealed that 10 μM UNBS5162 prevented PC-3 cell population development in vitro compared with control conditions. In addition, 10 μM UNBS5162 caused a marked enlargement in PC-3 cells by the end of the 6-day treatment period compared with the start of the experiment. Similar features were observed on treating DU-145 prostate cancer cells with 10 μM UNBS5162. However, at 1 μM, UNBS5162 induced no detectable changes in PC-3 and DU-145 cell dynamics as revealed by quantitative videomicroscopy.

Flow cytometry analysis revealed that treatment of PC-3 and DU-145 cells with 10 μM UNBS5162 for 72 hours markedly blocked PC-3 cells in their G2 cell cycle phase and to a lesser extent in DU-145 cells. Indeed, when treated with 10 μM UNBS5162, the percentage of PC-3 cells in the G₂/M phase markedly increased; accordingly, the percentage of cells in the G1 phase diminished. However, UNBS5162 at 1 μM did not significantly (P>0.05) modify PC-3 or DU-145 cell cycle kinetics. Furthermore, chronic treatment of PC-3 cells with 1 μM UNBS5162 for 5 days (“5×1”: cells treated in vitro for 24 hours with 1 μM UNBS5162 and the culture medium replaced by fresh medium containing 1 μM UNBS5162 every 24 hours for a total of 5 consecutive days) or 3 weeks (“3w1”: cells treated in vitro for 24 hours with 1 μM UNBS5162 and the culture medium replaced by fresh medium containing 1 μM UNBS5162 every 24 hours for a total of 3 consecutive weeks) did not notably modify PC-3 cell cycle kinetics. In addition to cell cycle arrest evidenced by flow cytometry, cellular imaging studies showed that UNBS5162 induced delayed growth and modified cellular morphology in human PC-3 and DU-145 prostate cancer cells, suggesting that this compound might be able to induce senescence; a permanent cell growth arrest. The literature reports that although specific mechanisms are as yet unknown, the senescence response seems to result in a reorganization of chromatin, at least some aspects of which require pRb activity. Replicatively, senescent cells develop dense foci of heterochromatin, which coincide with pRbdependent heterochromatic repression of genes encoding cyclins and other positive cell cycle regulators. Many of these repressed genes are activation targets of E2F transcription factors, some of which are converted to transcriptional repressors when complexed with pRb. The E2F family of transcription factors plays an important role in cell cycle progression. E2F-1, in heterodimeric complex with another protein, DP-1, is normally inactive because it is bound to hypophosphorylated pRb. When cells progress from the G1 to the S phase, pRb becomes hyperphosphorylated and releases the bound E2F-1/DP-1 heterodimer. Treatment of PC-3 cells with 10 μM UNBS5162 completely abolished Rb protein expression after 48 and 72 hours of treatment. This resulted in the complete dephosphorylation of pRb at the p^{Ser795} position and at positions p^{Ser780} and p^{Ser807/11}, with the further consequence of a dramatic decrease in E2F1 expression at both the protein and mRNA levels. Very similar features were observed in DU-145 prostate cancer cells, but less marked, particularly at the level of cell cycle kinetics and with respect to a decrease but not the complete disappearance of Rb protein and E2F1 expression. UNBS5162 at 1 μM induced no marked modifications in Rb, pRb, and E2F1 protein expression.

The enlargement of PC-3 cells revealed by quantitative videomicroscopy on treatment with 10 μMUNBS5162 prompted an investigation whether the compound at this concentration could induce senescence in these cells. Human PC-3 and DU-145 prostate cancer cells cultured in 0 or 10 μM UNBS5162 or 20 nM Adriamycin for 72 hours were evaluated by SA-β-Gal staining. The data clearly indicate that 10 μM [but not 1 μM or 5×1 μM] UNBS5162 induced marked expression of SA-β-Gal in DU-145 but not in PC-3 cells. “5×1 μM” indicates that tumor cells were treated in vitro for 24 hours with 1 μM UNBS5162 and the culture medium was replaced by fresh medium containing 1 μM UNBS5162 every 24 hours for a total of five consecutive days, with determination of senescence being performed 72 hours after the fifth treatment of cells with UNBS5162. TMZ, as an inducer of autophagy but not of senescence, was used as a negative control. Moderate concentrations of doxorubicin (ADR, 30 nM) induce senescence in wild type and in p53-mutated human cancer cells; accordingly, the compound was used as a positive control in our experiments and was found to be active at 20 and 50 nM. Limited SA-β-Gal expression was observed in PC-3 prostate cancer cells stimulated for 72 hours with either 10 μM UNBS5162 or with Adriamycin. A possible reason why PC-3 cells do not stain for SA-β-Gal is that p53 is deleted in these cells, whereas it is mutated in DU-145 cells.

In the process of identifying senescence-associated genes in prostate cancer cells, the data show that 72 hours after treatment with UNBS5162 at 10 μM but not at 1 μM, there was a marked sustained decrease in EHF mRNA levels in DU-145 but not in PC-3 prostate cancer cells. However, UNBS5162 at 1 μM markedly decreased EHF mRNA expression in a transient manner in DU-145 cells.

Amonafide and several analogues are known topoisomerase II inhibitors that induce apoptosis. We have already demonstrated that UNBS3157, the precursor of UNBS5162, is not a top( ) II poison but is a weak DNA-intercalating agent that does not induce apoptosis in prostate cancer cells. Furthermore, it is important to emphasize that the data received from the National Cancer Institute (NCI) clearly indicate that UNBS5162 and UNBS3157 have a markedly distinct profile to amonafide. In this study, using flow cytometry, we show that UNBS5162 does not induce real (early) apoptosis in PC-3 or in DU-145 cells. UNBS5162 induces late apoptotic and necrotic events in DU-145 cells that could have resulted from compound-induced proautophagic effects or senescence observed in this cell line. Indeed, using flow cytometry techniques for quantification of acidic vesicular organelles (autophagic vacuoles), it was possible to observe that UNBS5162 at 10 μM had a proautophagic effect in both cell lines. These cancer cell lines were then further evaluated to quantify the expression of light chain 3 cytosolic protein (LC3-I) and its processed light chain 3 membrane-bound form (LC3-II); a specific marker of autophagy. An immunoblot analysis technique was used to assess for autophagy as indicated by the LC3-II marker. UNBS5162 at 10 μM induced the up-regulation of LC3-II protein in the DU-145 cell line only; a feature that could partly explain why UNBS5162 induced weaker proautophagic effects in PC-3 cells. Although these data suggested that UNBS5162 induces autophagy-related effects in DU-145 and PC-3 cells, they did not confirm that UNBS5162 actually kills cancer cells by means of autophagy-related cell death. The possibility remained at this stage of our investigations that human prostate cancer cells might be defending themselves against the adverse effects of UNBS5162 by activating autophagy-related mechanisms of defense. Indeed, cells that undergo excessive autophagy are induced to die in a nonapoptotic manner, but cancer cells including human prostate cancer cells can also undergo autophagy to combat adverse events including chemotherapy and radiotherapy. The cellular imaging experiments described below strongly suggest that UNBS5162 does not kill PC-3 and DU-145 cells but rather irreversibly block their proliferation. Thus, the proautophagic effects observed in PC-3 and DU-145 cells when treating them with UNBS5162 correspond to autophagy-related defense mechanisms of these cell lines to the compound, rather than to actual UNBS5162-induced autophagy-related cell death. Furthermore, it is important to emphasize that chronic treatment of PC-3 cells with 1 μM UNBS5162 did not induce cell death either through apoptosis or autophagy-related processes.

In summary, at 10 μM UNBS5162 markedly impairs PC-3 tumor cell growth kinetics, without inducing senescence, whereas the reverse feature is observed with respect to DU-145 cells. The data is summarized in Table 9 below.

TABLE 9
Summary of 10 μM UNBS5162 Effects In Vitro
on Cell Proliferation and Cell Death.

| | PC-3 | DU-145 |
|---|---|---|
| | (p53 Null) | (p53 Mutated) |
| Growth arrest | | |
| Quantitative | ++ | + |
| videomicroscopy | | |
| G₂ phase blockade (FCM) | +++ | + |
| pRb expression (WB) | +++ | + |
| E2F1 expression (WB) | ++ | + |
| Senescence (SA-β-Gal) | − | +++ |
| Cell death | | |
| Apoptosis | − | − |
| Autophagy | − | + |

FCM indicates flow cytometry; WB, Western blot.

This difference might result from their respective p53 status and/or the extent of p16 expression, which is a positive regulator of pRb and tumor suppressor in its own right, as reported in the literature. At 1 μM, UNBS5162 induces no such antitumor effects. Thus, the data obtained in vitro when human prostate cancer cells are treated once with either 1 or 10 μM UNBS5162 cannot explain the activity obtained in vivo with the 10-mg/kg i.v. UNBS5162 regimen, which is likely to be associated with UNBS5162 plasma levels markedly less than 1 μM a short time after dosing. In contrast, chronic treatment with 5×1 μM UNBS5162 in vitro reconcile well with the data obtained in vivo. The presence or otherwise of active UNBS5162 metabolites in vivo has to be confirmed, and to this effect, an investigation of the compound's metabolism is currently ongoing.

Discussion

We recently reported that unlike amonafide, UNBS3157 does not display a mechanism of action characteristic of an intercalating agent. The NCI recently investigated UNBS3157 (coded as D-742814 by the NCI) and UNBS5162 (coded as D-742815) and compared their potential mechanism of action to those of approximately 750,000 compounds already available in their database. The NCI concluded that, whereas the mechanisms of action of UNBS3157 and UNBS5162 were quite comparable (correlation coefficient 0.78); they were distinct from those of the 750,000 compounds (unpublished data). The NCI 60 Cell Line Panel analysis indicated that UNBS3157 and UNBS5162 might have the profile of a multidrug resistance P-glycoprotein (MDR-Pgp) substrate. On investigation, it has been confirmed that even at 100 μM, UNBS3157 and UNBS5162 do not affect Pgp ATPase activity (data not shown).

Affymetrix genome-wide microarray analysis and ELISAs have revealed that in vitro incubation of UNBS5162 (1 μM five times a week) with human PC-3 prostate cancer cells dramatically decreased (at both mRNA and protein levels) the expression of the proangiogenic CXCL1, CXCL2, CXCL3, CXCL5, CXCL6, and CXCL8 chemokines, whereas acute administration of 10 μM did not. Data obtained in PC-3 cells were reproduced in DU-145 cells. Histopathologic analysis additionally revealed anti-angiogenic properties in vivo for UNBS5162 in the orthotopic PC-3 model.

It should be recalled that a complex network of chemokines and their receptors influences the development of primary tumors and metastases. Chemokine signaling results in the transcription of target genes that are involved in cell invasion, motility, interactions with the extracellular matrix, and survival of both normal and cancer cells. The small (8-10 kDa) chemokines are classified into four highly conserved groups, namely, CXC, CC, C, and CX3C, based on the position of the first two cysteines that are adjacent to the amino terminus. More than 50 chemokines have been discovered so far, and there are at least 18 human seven-transmembrane domain chemokine receptors. CXC chemokines are a unique cytokine family that exhibit, on the basis of structure/function and receptor binding/activation, either angiogenic or angiostatic biologic activity in the regulation of angiogenesis. The glutamic acid-leucine-arginine (ELR+) CXC chemokines [such as GRO-α/N51/KC(CXCL1), Gro-β/MIP-2α (CXCL2), and IL-8 (CXCL8)] are potent promoters of angiogenesis and mediate their angiogenic activity through signal coupling of CXCR2 on the endothelium. The proangiogenic members of the CXC chemokines are directly chemotactic to endothelial cells and cancer cells, which display the receptors for these CXCL chemokines, and they stimulate angiogenesis in vivo. By contrast, members of the CXC chemokine family (ELR.), such as platelet factor-4 (PF4; CXCL4) and interferon-inducible CXC chemokines, are potent inhibitors of angiogenesis and use CXCR3 on the endothelium to mediate their angiostatic activity. A number of studies have demonstrated that proangiogenic chemokines mediate the tumorigenicity of prostate cancer cells, due at least partly to constitutively activated nuclear factor-κB/p65 (Rel A) in human prostate adenocarcinoma, as reported in the literature. Also, it has been demonstrated that CXCL8 is not detectable in androgen-responsive prostate cancer cells but is highly expressed in androgen-independent metastatic cells, and it functions in androgen independence, tumor growth, chemoresistance, metastases, and angiogenesis. Furthermore, CXCL1, CXCL3, CXCL5, and CXCL6 also directly influence the biologic behavior of human prostate cancer cells. As revealed in the present study and by additional unpublished data from our laboratory, CXCL9, 10, and 11, which exert rather antiangiogenic effects, are not expressed or are only very weakly expressed in human PC-3 and DU-145 prostate cancer cells. In contrast, the data from the present study show that CXCL1, CXCL2, CXCL3, CXCL6, and CXCL8 are expressed at very high basal levels in human prostate cancer cells and that UNBS5162 administered in vitro in a metronomic approach almost completely abolished their expression, with impairment of in vivo angiogenesis as a consequence. The fact that the antitumor effects of UNBS5162 are more pronounced when administered repeatedly at low doses rather than acutely at high doses must be considered in the light of the studies published by Kerbel et al. with respect to the fact that metronomic chemotherapy can actually be more effective than high dose monotherapy. The present study demonstrates that metronomic delivery of a compound, i.e., UNBS5162, even in vitro, targets clusters of genes that are totally different to those targeted by an acute high dose of the same compound. Repeat in vivo i.v. administration of UNBS5162 despite an apparent low plasma drug exposure after 1 to 2 hours also markedly increased the therapeutic benefits of taxol.

Chemokines (including CXCL chemokines) and their receptors are involved in malignant progression, and a better understanding of chemokine signaling in this process could lead to new therapeutic strategies for cancer. As the chemokine network is complex, it is unlikely that an individual chemokine antagonist would have a sufficiently powerful action in cancer. Small-molecule antagonists exist for several chemokine receptors. The present study shows that UNBS5162 is a pan-antagonist of CXCL chemokine expression that displays antitumor effects in experimental models of human refractory prostate cancers. The manner in which UNBS5162 antagonizes CXCL chemokine expression remains unknown, but the present study strongly suggests that this antagonism does not occur at the level of CXCL chemokine receptors.

### Example 15N-{2-[2-(dimethylamino)ethyl]-1,3-dioxo-2,3-dihydro-1H-benzo[de]isoquinolin-5-yl}urea (UNBS5162) in Esophageal Cancer

In Example 15, UNBS5162 was studied to evaluate the efficacy and the subsequent mode of action in esophageal cancer. The effects mediated by UNBS5162 in vitro and in vivo in the models of esophageal cancer were analyzed. The data are presented below.

### Rationale:

Data obtained on prostate cancer models revealed UNBS5162 as potent inhibitor of proangiogenic CXC chemokines and CCL2 chemokine (Mijatovic et al., Neoplasia 2008.

It has been reported that many cancers have a complex chemokine network affecting the transcription of target genes involved in cell invasion, motility, interactions with the extracellular matrix and the survival of cancer cells.

Among others, Wang et al. (Cancer Research 66, 2006) reported that CXCLI-CXCR2 and CXCL2-CXCR2 signalling contributes significantly to esophageal cancer cell proliferation and that this autocrine signalling pathway may be involved in esophageal tumorigenesis.

Data obtained on different xenograft nude mice models indicated that combining conventional chemotherapy with UNBS5162 markedly improves the survival of tumor-bearing mice (Van Quaquebeke et al., J Med Chem 2007 [appendix no. 1]; Mijatovic et al., Neoplasia 2008 [appendix no. 2]; UNBS5162-NSCLC report; UNBS5162-glioma report).

Collectively, our results obtained to date emphasize the role of proangiogenic chemokines in esophageal cancer biology and bring a novel proposal for combine therapy involving chemokine inhibition to improve clinical outcomes.

### Results

The Expression Status and Role of CXCLs and CCL2 Chemokines and their Receptors in Esophageal Cancer

The expression status and role of CXCLs and CCL2 chemokines and their receptors in esophageal cancer have remained undefined. Accordingly, using standard and quantitative RT-PCR methods, the expression of chemokines and their receptors was examined in two established cell lines (OE21 [ECACC code 96062201] and OE33 [ECACC code 96070808]) and in four samples of esophageal cancer resection. The primers and the PCR conditions for each target evaluated in the present study are set forth in Table 10 below.

TABLE 10

The primers and the PCR conditions for each target evaluated
in the present study

| | | | |
|---|---|---|---|
| | | Fragment | Annealing |
| Name | Primer sequence | size | temperature |

| | | | | |
|---|---|---|---|---|
| CXCL1 | Forward | 5′-agggtatgattaactctacctg-3′ | 407 pb | 57.3° C. |
| | Reverse | 5′-ccattaaacaaggcagtatgc-3′ | | |
| CXCL2 | Forward | 5′-gtcaaacccaagttagttca-3′ | 340 pb | 57° C. |
| | Reverse | 5′-cagtatgccttacaagaaagac-3′ | | |
| CXCL3 | Forward | 5′-agcttatcagcgtatcattgac-3′ | 391 pb | 58.2° C. |
| | Reverse | 5′-ccctaacagtgatccactaa-3′ | | |
| CXCL5 | Forward | 5′-agagtagaacctgggttaga-3′ | 333 pb | 58° C. |
| | Reverse | 5′-cctacaagccttttcacaag-3′ | | |
| CXCL6 | Forward | 5′-ttgaaccctttggcaattg-3′ | 262 pb | 57.5° C. |
| | Reverse | 5′-gggtaaagagtaacatattccc-3′ | | |
| CXCL7 | Forward | 5′-cttgtaggcagcaactca-3′ | 249 pb | 58° C. |
| | Reverse | 5′-gcatacaagtcactgtctaga-3′ | | |
| CXCL8 | Forward | 5′-tgggtgcagagggttgtg-3′ | 526 pb | 60° C. |
| | Reverse | 5′-cagactagggttgccagattta-3′ | | |
| CXCL9 | Forward | 5′-gctttctaagatctaacaagatagc-3′ | 406 pb | 58.2° C. |
| | Reverse | 5′-ggaactagggagtttcatga-3′ | | |
| CXCL10 | Forward | 5′-atgaatcaaactgcgattctgatt-3′ | 296 pb | 58° C. |
| | Reverse | 5′-ttaaggagatcttttagacatttc-3′ | | |
| CXCL11 | Forward | 5′-ggttaccatcggagtttaca-3′ | 332 pb | 58.8° C. |
| | Reverse | 5′-ccctacatattgatgtgctacatg-3′ | | |
| CXCL12 | Forward | 5′-atgaacgccaaggtcgtggtc-3′ | 266 pb | 60° C. |
| | Reverse | 5′-cttgtttaaagctttctccaggtact-3′ | | |
| CXCL13 | Forward | 5′-ccctagacgcttcattga-3′ | 322 pb | 60° C. |
| | Reverse | 5′-ctcatgccttatttgtatggg-3′ | | |
| CXCL14 | Forward | 5′-aagcttccgcttagaggt-3′ | 367 pb | 60° C. |
| | Reverse | 5′-cctaaggtttttgctgacagt-3′ | | |
| CXCL 16 | Forward | 5′-ctgactcagocaggcaatgg-3′ | 379 pb | 55° C. |
| | Reverse | 5′-tgagtggactgcaaggaaggtgga-3′ | | |
| CXCR1 | Forward | 5′-ggctgctggggactgtctatgaat-3′ | 382 pb | 57° C. |
| | Reverse | 5′-gcccggccgatgttgttg-3′ | | |
| CXCR2 | Forward | 5′-gtaattacagttacagctctaccc-3′ | 517 pb | 60° C. |
| | Reverse | 5′-gctaacattggatgagtagacg-3′ | | |
| CXCR3 | Forward | 5′-tggacatcctcatggacctg-3′ | 319 pb | 62° C. |
| | Reverse | 5′gaagtcagactgtgggcgaa-3′ | | |
| CXCR4 | Forward | 5′-atcttcctgcccaccatctactccatcatc-3 | 370 pb | 57° C. |
| | Reverse | 5′-atccagacgccaacatagaccaccttttca-3′ | | |
| CXCR5 | Forward | 5′-agctatagacccgaggaa-3′ | 463 pb | 57.5° C. |
| | Reverse | 5′-agcttgcgaggagatact-3′ | | |
| CXCR6 | Forward | 5′-gtcatatccatcttctaccataagt-3′ | 401 pb | 58.8° C. |
| | Reverse | 5′-aattgcctcgtcatggtaa-3′ | | |
| KSHV | Forward | 5′-tgttaccttctgaaactgtacc-3′ | 295 pb | 60° C. |
| | Reverse | 5′-ggtgtaaattcaggagaaatcg-3′ | | |
| DUFFY | Forward | 5-cttcctatggtgtgaatgattc-3′ | 180 pb | 57.5° C. |
| | Reverse | 5′-aagagaggtctgaaaagcat-3′ | | |
| CCL2 | Forward | 5-taacccagaaacatccaattc-3′ | 402 pb | 57° C. |
| | Reverse | 5′-gctaggggaaaataagttagc-3′ | | |
| CCR2 | Forward | 5′-gagtcaacccaatagttgttg-3′ | 226 pb | 60° C. |
| | Reverse | 5′-acactcgaatgtgattaaacg-3′ | | |

Using standard PCR technique based on these conditions, we were able to determine the pattern of the expression of chemokines and their receptors in two established human esophageal cancer cell lines (OE21 [ECACC code 96062201] and OE33 [ECACC code 96070808]) and in four samples of human esophageal cancer resection (CSI-4).

Total RNA was extracted using the TRIzol isolation reagent (Life Technologies, Inc., Merelbeke, Belgium) according to the manufacturer's instructions. The RNA extracted was treated with DNase I (Life Technologies, Inc.) to eliminate any remaining genomic DNA. All reverse transcription and PCR reactions were carried out in a thennal cycler (Thermocycler, Westburg, Leusden, The Netherlands). The purification of the cDNAs produced was carried out using the High Pure PCR Product Purification Kit (Roche Diagnostics, Mannheim, Germany) in accordance with the manufacturer's instructions. The integrity of the cDNA was confirmed by an analysis of B-actin gene expression. All of the PCR analyses were performed on the basis of the same quantity of purified cDNA (total amount 20 ng).

TABLE 11
Qualitative determination of mRNA expression levels for CXCLs
and CCL2 chemokines and their receptors (CXCL-Rs and CCR2)
in 4 human clinical samples of esophagus cancers and 2 human
established cell lines from esophagus cancers

| CXCL | CXCL-R | CS1 | CS2 | CS3 | CS4 | OE21 | OE33 |
|---|---|---|---|---|---|---|---|
| CXCL1 | | + | ++ | ++ | ++ | + | + |
| | CXCR2 | − | ++ | ++ | ++ | ++ | + |
| | KSHV | + | + | + | + | + | + |
| | Duffy | + | + | + | + | − | − |
| CXCL2 | | ++ | ++ | ++ | ++ | ++ | ++ |
| CXCL3 | | − | − | − | − | − | + |
| CXCL5 | | − | ++ | + | + | − | + |
| CXCL6 | | − | + | + | + | − | + |
| | CXCR1 | − | + | + | − | − | − |
| CXCL7 | | + | + | + | + | − | ++ |
| CXCL8 | | ++ | ++ | ++ | ++ | ++ | ++ |
| CXCL9 | | ++ | ++ | ++ | ++ | + | − |
| | CXCR3 | − | ++ | + | ++ | + | − |
| CXCL10 | | − | − | − | − | − | − |
| CXCL11 | | + | ++ | + | + | + | + |
| CXCL12 | | + | ++ | + | − | ++ | ++ |
| | CXCR4 | ++ | ++ | + | − | − | − |
| CXCL13 | | + | ++ | ++ | ++ | + | − |
| | CXCR5 | + | + | + | + | ++ | − |
| CXCL14 | | − | − | ++ | ++ | ++ | − |
| CXCL16 | | ++ | ++ | + | ++ | ++ | ++ |
| | CXCR6 | + | + | − | ++ | ++ | + |
| CCL2 | | ++ | ++ | ++ | ++ | ++ | + |
| | CCR2 | + | ++ | + | ++ | + | − |

−: negative,
+: weakly positive;
++: positive

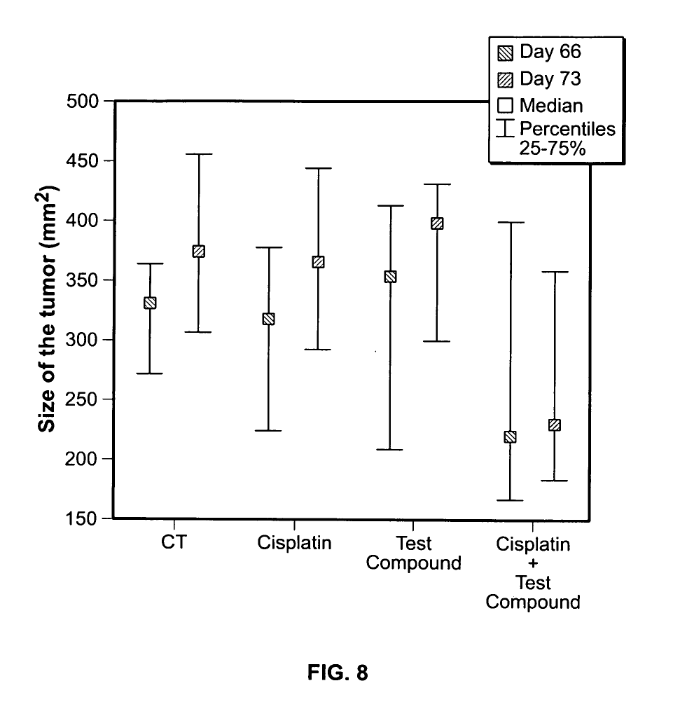

These analyses revealed a highly similar expression pattern in the cell lines and the surgical specimens. Chemokines and receptors for which RNA expression was evidenced by means of standard PCR were further analyzed by means of quantitative RT-PCR according to the methodology fully described in Lefranc et al., Neuropathol Appl Neurobiol. 31; 2005. These results are presented on FIG. 8A (chemokines) and 2B (receptors).

These analyses revealed a marked over-expression of pro-angiogenic chemokines, especially CXCL-I, CXCL-2 and CXCL-8. The involvement/role of these chemokines and their receptors in esophageal cancer is being further investigated using a siRNA approach followed by cellular imaging analysis (real-time video-microscopy) in order to evidence the effect of knocking-down the expression of these proteins.

It should be recalled that a complex network of chemokines and their receptors influences the development of primary tumors and metastases (Balkwill Nat. Rev. Cancer 2004; Fernandez & Lolis Annu. Rev. Pharmacol. Toxicol 2002). Chemokine signalling results in the transcription of target genes that are involved in cell invasion, motility, interactions with the extra-cellular matrix and survival of both normal as well as cancer cells (Balkwill Nat. Rev. Cancer 2004; Fernandez & Lolis Annu. Rev. Pharmacol. Toxicol 2002). CXC chemokines are a unique cytokine family that exhibit, on the basis of structure/function and receptor binding/activation, either angiogenic or angiostatic biological activity in the regulation of angiogenesis (Strieter et al., Eur. J. Cancer 2006). The glutamic acid-leucine-arginine (ELR+) CXC chemokines (such as GRO-α/N51/KC(CXCL1), Gro-β/MIP-2α (CXCL2), IL-8 (CXCL8)) are potent promoters of angiogenesis, and mediate their angiogenic activity via signal-coupling of CXCR2 on the endothelium. By contrast, members of the CXC chemokine family (ELR−), such as platelet factor-4 (PF4; CXCL4) and interferon-inducible CXC chemokines are potent inhibitors of angiogenesis, and use CXCR3 on the endothelium to mediate their angiostatic activity.

Our data obtained in the study involving prostate cancer cells showed that CXCL1, CXCL2, CXCL3, CXCL6 and CXCL8 are expressed at very high basal levels in human prostate cancer cells and that UNBS5162 administered in vitro in a metronamic approach almost completely abolished their expression, with as a consequence impairment of in vivo angiogenesis. In line with this, we aimed to analyse if UNBS5162 is able to, down-regulate CXCL-1 and CXCL-8 expression in human esophageal cancer cells.

### CXCL Chemokine Down-Regulation Mediated by UNBS5162

Using UNBS5162, it was possible to demonstrate by means of an ELISA assay, a marked decrease in CXCL-1 and CXCL-8 in OE21 cells.

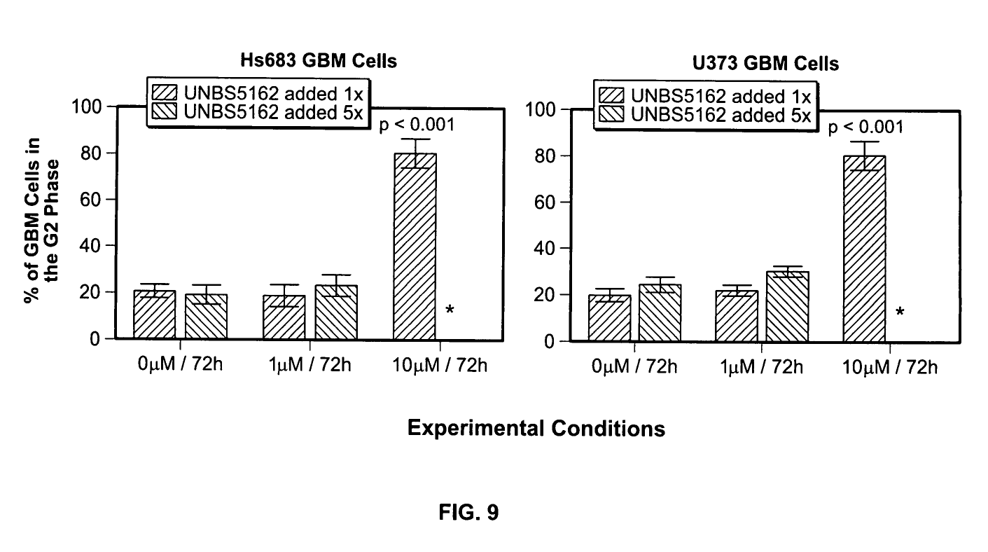

ELISA determination of CXCL1 and CXCL8 (IL-8) protein levels in untreated and 5×1 μM UNBS5162-treated OE21 cells. “5×1 μM” means that tumor cells were treated in vitro for one day with 1 μM UNBS5162 and then the culture medium was replaced by fresh medium containing 1 μM UNBS5162; this procedure of renewal of cell medium containing 1 μM UNBS5162 was repeated for five consecutive days, with determination of chemokine concentration being performed 24 h (+24 h) or 72 h (+72 h) after the 5th (last) treatment of tumor cells with UNBS5162. The concentrations of the two specific chemokines (expressed as pg/mL) were normalized by the cell number determined in each sample. Each sample was assessed in triplicate. The data are presented as means (grey columns)±SEM (thin bars) in FIG. 9.

UNBS5162 Alone and in Combination with Cisplatin Tested for In Vivo Anti-Tumor Effects in S.C. Metastatic OE21 Xenograft Nude Mice Model

To enable the achievement of pertinent in vivo studies, a highly metastatic s.c. model was developed by grafting OE21 cells subcutaneously into nude mice. Data obtained on different xenograft nude mice models indicated that combining conventional chemotherapy with UNBS5162 markedly improves the survival of tumor-bearing mice. In line with this, an experiment combining cisplatin to UNBS5162 was performed.

The treatment protocol included following conditions:

UNBS5162: N-{2-[2-(dimethylamino)ethyl]-1,3-dioxo-2,3-dihydro-1H-benzo[de]isoquinolin-5-yl}urea

### Route: i.v.

Dose (mg/kg): 10 mg/kg (1.35 eq lactic acid)
Dose volume: 200 μL /mouse
Schedule: 3 injection(s) per week; 5 weeks (3i×5w)

### Compound Cisplatine:

### Route: i.p.

Dose (mg/kg): 5 mg/kg (saline)
Dose volume: 200 μL/mouse
Schedule: 1 injection per week; 5 weeks (1×5)

### Groups:

### Control (5×5)=>CTRL

Cisplatine (1×5)×5 mg/kg i.p.=>group 1
UNBS5162 (3×5)×10 mg/kg i.v.=>group 3
Cisplatine (1×3)×5 mg/kg i.p.=>1 w without treatment=>UNBS5162 (3×5)×to mg/kg i.v.=>group

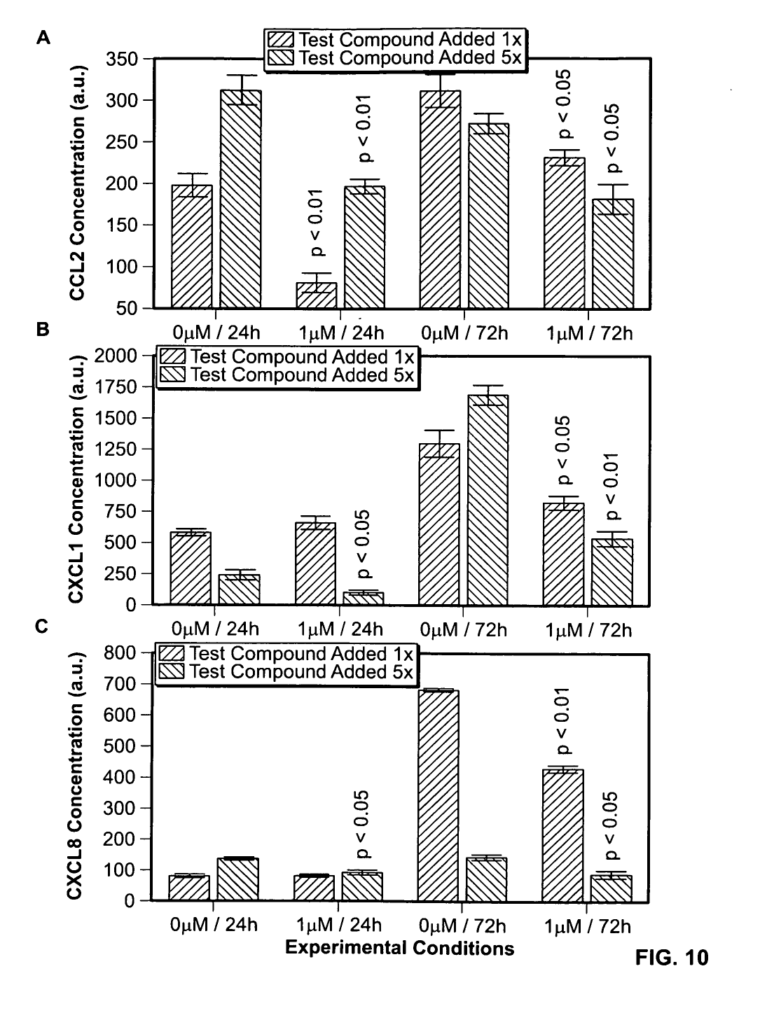

As shown on FIG. 10, treatment of these mice with UNBS5162 enhanced the activity of cisplatin when the two compounds were co-administered, as evidenced by a decrease in tumor size.

In order to strengthen the correlation between chemokine expression and anti-tumor activity in vivo, immunostaining of excised tumors is presently ongoing and comprise labeling for CXCL-I, CXCL-2 and CXCL-8.

In order to further improve therapeutic benefit in this aggressive model, a novel set of experiments is now being conducted combining different chemotherapeutic agents with UNBS5162. Our preliminary results indicated that esophageal tumors might better respond to pro-autophagic rather than to pro-apoptotic treatment. In line with this, a new experiment has been designed involving the combinations between UNBS5162 with pro-autophagic compounds such as Temodal (Kanzawa et al., Cell Death Differ. 2004; Lefranc et al., Oncologist. 2007) and Dacarbazine.

The treatment protocol includes following conditions:

Temodal (Temodal 250 mg; Schering Plough, Belgium):
Vehicle: saline
Route: p.o.
Dose (mg/kg): 80 mg/kg
Dose volume: 200 μL/mouse
Schedule: 3 injections/week; 3 or 9 weeks (3i×3w) or (3i×9w)

Dacarbazine (Dacarbazine Medac 500 mg; PCR Pharmachemie, Teva Pharma, Belgium):
Vehicle: saline
Route: i.p.
Dose (mg/kg): 80 mg/kg
Dose volume: 200 μL /mouse
Schedule: 3 injections/week; 3 or 9 weeks (3i×3w) or (3i×9w)

UNBS5162:
Vehicle: 1.35 eq lactic acid (L6661-500mL, Sigma-Aldrich)
Route: Lp.
Dose (mg/kg): 10 mg/kg (1.35 eq lactic acid)
Dose volume: 200 μL /mouse
   Schedule: 3 injections/week; 3 or 9 weeks (3i×3w) or (3i×9w)

### Groups:

Control (3i×9w)=>CTRL
Temodal D14 (3i×9w)×80 mg/kg p.o.=>group 1
Dacarbazine D14 (3i×9w)×80 mg/kg i.p.=>group 2
UNBS5162 D14 (3i×9w)×10 mg/kg i.p.=>group 3
Temodal D14 (3i×3w)=>UNBS5162 035 (3i×3w)=>UNBSI450 056 (3i×3w)=>group 4
Dacarbazine D14 (3i×3w)=>UNBS5162 035 (3i×3w)=>UNBSI450 056 (3i×3w)=>group 5
Temodal D14 (3i×3w)=>UNBS1450 035 (3i×3w)=>UNBS5162 056 (3i×3w)=>group 6
Dacarbazine D14 (3i×3w)=>UNBSI450 035 (3i×3w)=>UNBS5162 056 (3i×3w)=>group 7

### Parameters to be Monitored:

Body weight (3 times per week)
Tumor size (2 times per week)
Table of sacrifice or death

### Data Analysis:

Statistical comparisons between the control group and the treated groups are performed with the non-parametric Mann-Whitney U-test. Survival analysis is done either by calculating the % T/C-value or either with Kaplan-Meier analysis and Log-rank statistics. All statistical analyses are carried out using Statistica software (Statsoft, Tulsa, Okla.). The cut-off level for statistical significance is set at p-value ofp <0.05.

### Histology:

Tumor+liver+lungs are carefully isolated, processed in paraffin blocs and stored before histological analysis.

Comparable findings as found in the above examples are expected.

### Example 16

### UNBS5162 N-{2-[2-(dimethylamino)ethyl]-1,3-dioxo-2,3-dihydro-1H-benzo[de]isoquinolin-5-yl}urea

as potential treatment for glioblastoma (GBM) and gliosarcoma was studied.

Rationale:

Invasive GBM cells show an increase in proliferation rates and a resistance to apoptosis. Despite this resistance to apoptosis being closely linked to tumorigenesis, tumor cells can still be induced to die by non-apoptotic mechanisms such as autophagy (Lefranc et al., J. Clin. Oncol 2005; The Oncologist 2007)

Data obtained on prostate cancer models revealed that UNBS5162, when used at single dose of 10 μM, induces cell cycle arrest in G2 phase and induces rather senescence and proautophagic cell death than apoptosis (Mijatovic et al., Neoplasia 2008).

The mechanism of action of UNBS5162 depends on the administration scheme, i.e. when administered repeatedly at low doses (5×1 μM) or acutely at high doses (10 μM) (Mijatovic et al., Neoplasia 2008).

Data obtained on prostate cancer models revealed UNBS5162 as potent inhibitor of proangiogenic CXC chemokines and CCL2 chemokine when used repeatedly at 1 μM, mimicking thus chronic drug administration (Mijatovic et al., Neoplasia 2008).

It has been reported that many cancers have a complex chemokine network affecting the transcription of target genes involved in cell invasion, motility, interactions with the extracellular matrix and the survival of cancer cells.

Data obtained on different xenograft nude mice models indicated that combining conventional chemotherapy with UNBS5162 markedly improves the survival of tumor-bearing mice (Van Quaquebeke et al., J Med Chem 2007; Mijatovic et al., Neoplasia 2008).

Recent reports include demonstrating chemokine involvement in radioresistance and their upregulation by radiotherapy (Bastianutto et al. Cancer Res. 67, 2007; Klopp et al., Cancer Res. 67, 2007). Tamatani et al. (Int J Oncol 31, 2007; Int J Cancer. 108, 2004) demonstrated that that the combination of radiotherapy and cepharanthin (a biscoclaurine alkaloid extracted from the roots of Stephania cepharantha hayata reported to be able to inhibit NF-kappa B activation and IL-6 and IL-S (CXCL8) production) could enhance radiosensitivity in the treatment of human oral cancer.

Data obtained on the syngeneic MXT-HI mammary tumor after subcutaneous inoculation into conventional mice revealed that irradiation (1×10 gray) combined with UNBS5162 treatment (10 mg/kg i.v.; 5i×3w) significantly increased the survival of subcutaneous MXT-HI-bearing mice (Kaplan-Meier (Log-rank statistics) analysis: p=0.001).

Results

UNBS5162 at 10 uM Induces Cell Cycle Arrest in G2 Phase and Provokes Rather Pro-Autophagic Cell Death than Apoptosis in Human Glioblastoma Cell Lines

The cell cycle kinetics of glioma Hs683 (A TCC code HTB-138) and U373 (A TCC code HTB-17) cells left untreated or incubated with UNBS5162 as indicated were determined by flow cytometry analysis of propidium iodide (PI) nuclear staining, using previously detailed methodology (Mijatovic et al. Neoplasia 2006, 2008). Each sample was evaluated in triplicate. Flow cytometry was undertaken using an Epics XL.MCL flow cytometer and the F ACScan/CellQuest software system (Becton Dickinson, Miami, Fla., USA).

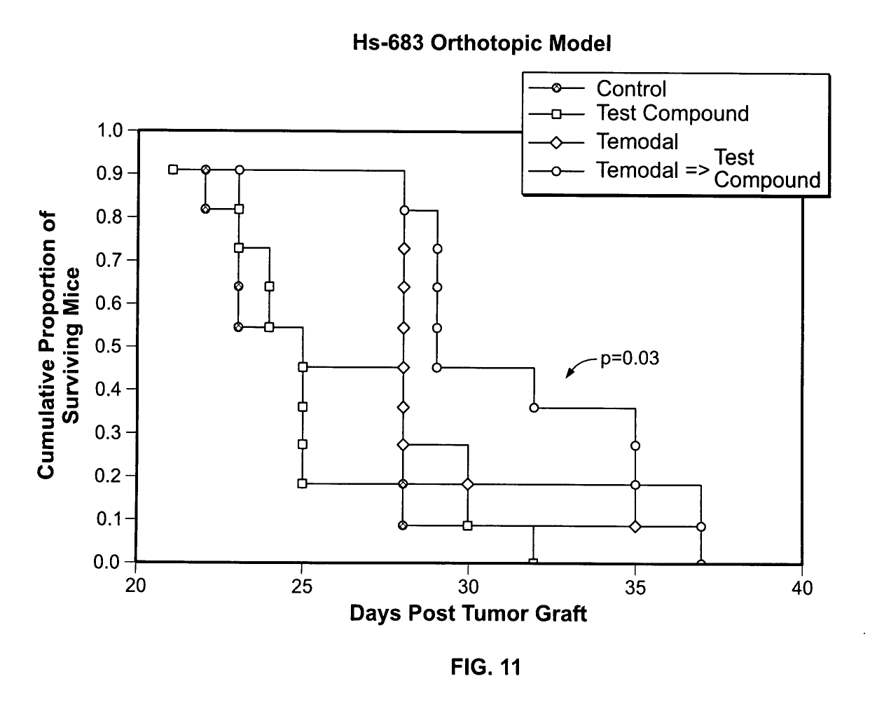

This analysis revealed that treatment of glioma cells with 10 μM UNBS5162 for 72 h markedly blocked both Hs683 and U373 cells in their G2 cell cycle phase. Conversely, UNBS5162 at 1 μM did not significantly modify glioma cell cycle kinetics. Furthermore, chronic treatment of glioma cells with 1 μM UNBS5162 for five days did not notably modify glioma cell cycle kinetics. Finally, chronic treatment of glioma cells with 10 μM UNBS5162 for five days induced the death of all assayed cells before the end of experiment. The results are set forth in FIG. 11.

UNBS5162 Induces Apoptosis in Glioma Cells Only when Assayed at 10 μM for 72 h

UNBS5162-induced apoptotic effects as evaluated by flow cytometry with Annexin V-FITC staining of Hs683 or U373 cells either left untreated (0 μM) or treated with 1 and 10 μM UNBS5162 for 72 h, either as a single treatment (gray bars) or as repeated (“chronic”; black bars) treatment (i.e. 1 μM or 10 μM each day for five days, “5×1”). The data are presented as means±SEM. “*” means that all GBM cells died before the end of the experiment.

UNBS5162 induces moderate apoptotic events in glioma cells only when they have been treated with 10 μM for 72 h. These apoptotic events could have resulted from UNBS5162-induced pro-autophagic effects or senescence observed in these cell lines (Lefranc et al., EANO 2008).

The expression status of CXCLs and CCL2 chemokines and their receptors in glioblastoma cells has remained undefined. Accordingly, using standard and quantitative RT-PCR methods, the expression of chemokines and their receptors was examined in four established human glioma cell lines [Hs683 (ATCC code HTB-138), U373 (ATCC code HTB-17), T98G (ATCC code CRL-1690) and U87 (ATCC code HTB-14)], four glioblastoma primocultures we established from surgical resections performed by Dr F. Lefranc at Erasmus University Hospital, Brussels, Belgium [GBM-PS, GBM-P16, GBM-P17, GBM-P19], and in eleven samples of glioma resection [three samples of normal brain (NI, N2, N3) and eight glioblastoma samples (GBM1-8)].

Using standard PCR technique based on conditions detailed in Table 10 above, we were able to determine the pattern of the expression of chemokines and their receptors in four established human glioma cell lines and in four glioblastoma primocultures established from surgical resections.

Total RNA was extracted using the TRIzol isolation reagent (Life Technologies, Inc., Merelbeke, Belgium) according to the manufacturer's instructions. The RNA extracted was treated with DNase I (Life Technologies, Inc.) to eliminate any remaining genomic DNA. All reverse transcription and PCR reactions were carried out in a thermal cycler (Thermocycler, Westburg, Leusden, The Netherlands). The purification of the cDNAs produced was carried out using the High Pure PCR Product Purification Kit (Roche Diagnostics, Mannheim, Germany) in accordance with the manufacturer's instructions. The integrity of the cDNA was confirmed by an analysis of β-actin gene expression. All of the PCR analyses were performed on the basis of the same quantity of purified cDNA (total amount 20 ng).

The results are summarized in the Table 12 below.

TABLE 12
Qualitative determination of mRNA expression levels for CXCLs
and CCL2 chemokines and their receptors (CXCL-Rs and CCR1)
in four established human glioma cell lines and in four
glioblastoma primocultures established from surgical resections

| | | GBM- | GBM- | GBM- | GBM- | | | | |
|---|---|---|---|---|---|---|---|---|---|
| CXCL | CXCL-R | PS | P16 | P17 | P19 | Hs683 | T98G | U87 | U373 |
| CXCL1 | | ++ | ++ | − | − | ++ | − | ++ | − |
| | CXCR1 | + | − | + | + | + | − | + | − |
| | KSHV | + | − | + | + | + | + | + | + |
| | DUFFY | − | + | − | − | + | − | − | − |
| CXCL2 | | ++ | + | − | ++ | ++ | ++ | ++ | ++ |
| CXCL3 | | + | + | − | + | ++ | + | + | + |
| CXCL4 | | − | − | − | − | − | − | − | − |
| CXCL5 | | − | ++ | − | + | ++ | + | ++ | − |
| CXCL6 | | + | − | − | − | − | − | − | − |
| | CXCRI | − | − | − | − | ++ | − | − | − |
| CXCL7 | | − | − | − | − | ++ | − | ++ | − |
| CXCL8 | | ++ | ++ | ++ | ++ | ++ | ++ | ++ | ++ |
| CXCL9 | | − | − | − | − | − | − | − | − |
| | CXCR3 | − | − | − | − | + | − | − | − |
| CXCL10 | | − | + | − | − | + | − | + | − |
| CXCL11 | | − | ++ | + | − | + | − | + | − |
| CXCL12 | | | + | + | + | + | + | ++ | + |
| | CXCR4 | ++ | ++ | ++ | + | ++ | − | − | ++− |
| CXCL13 | | − | − | − | − | − | − | − | − |
| | CXCR5 | − | − | − | − | − | − | − | − |
| CXCL14 | | − | − | + | − | − | ++ | + | + |
| CXCL16 | | ++ | + | − | ++ | ++ | ++ | − | ++ |
| | CXCR6 | − | − | + | − | − | − | + | − |
| CCL2 | | + | ++ | ++ | ++ | ++ | ++ | ++ | ++ |
| | CCR2 | − | − | − | + | − | − | + | + |

−: negative,
+: weakly positive;
++: positive by PCR

These analyses revealed a heterogeneous expression pattern both among established cell lines as well as among primocultures. Chemokines for which RNA expression was evidenced by means of standard PCR were further analyzed by means of quantitative RT-PCR according to the methodology fully described in Lefranc et al., Neuropathol Appl Neurobiol. 31; 2005 These analyses revealed that (i) primocultures express less CXCL-1,-2,-3 and -8 that established cell lines, (ii) among the established cell lines, U373 express very low levels of investigated chemokines, (iii) in contrast to surgical specimens from normal brain, glioblastomas express high levels of CXCL chemokine RNA.

This analysis evidenced very high CCL2 mRNA levels in all tested glioma cells, in contrast to CCR2, detected only in Hs683 and T98G cell lines.

As already emphasized, data obtained in the study involving prostate cancer cells showed that CCL2, CXCL 1, CXCL2, CXCL3, CXCL6 and CXCL8 are expressed at very high basal levels in human prostate cancer cells and that UNBS5162 administered in vitro in a metronomic approach almost completely abolished their expression, with as a consequence impairment of in vivo angiogenesis (Mijatovic et al., Neoplasia 2008). In line with this, a study was conducted to determine if UNBS5162 is able to down-regulate CCL2, CXCL-I and CXCL-8 expression in human glioma cells.

UNBS5162-Mediated CXCL Chemokine Down-Regulation

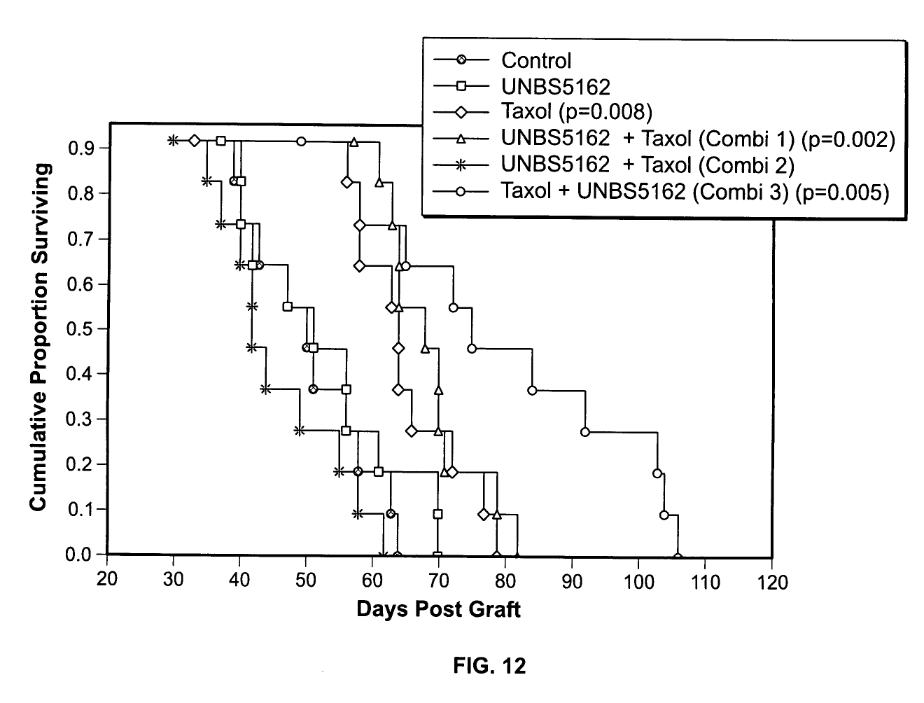

Using UNBS5162, it was possible to demonstrate by means of an ELISA assay, a marked UNBS5162-mediated decrease in CCL2, CXCL-I and CXCL-8 production in Hs683 cells. The results are set forth in FIG. 12.

UNBS5162-Mediated In Vivo Anti-Tumor Effects in Orthotopic Hs683 Xenograft Nude Mice model—Glioma Cells

In order to evaluate UNBS5162-mediated in vivo anti-tumor effect in glioma models, an orthotopis Hs683 model was used that was previously evidenced as expressing pro-angiogenic chemokines at the most high level. Data obtained on different xenograft nude mice models indicated that combining conventional chemotherapy with UNBS5162 markedly improves the survival of tumor-bearing mice. In line with this, an experiment combining Temodal to UNBS5162 was performed.

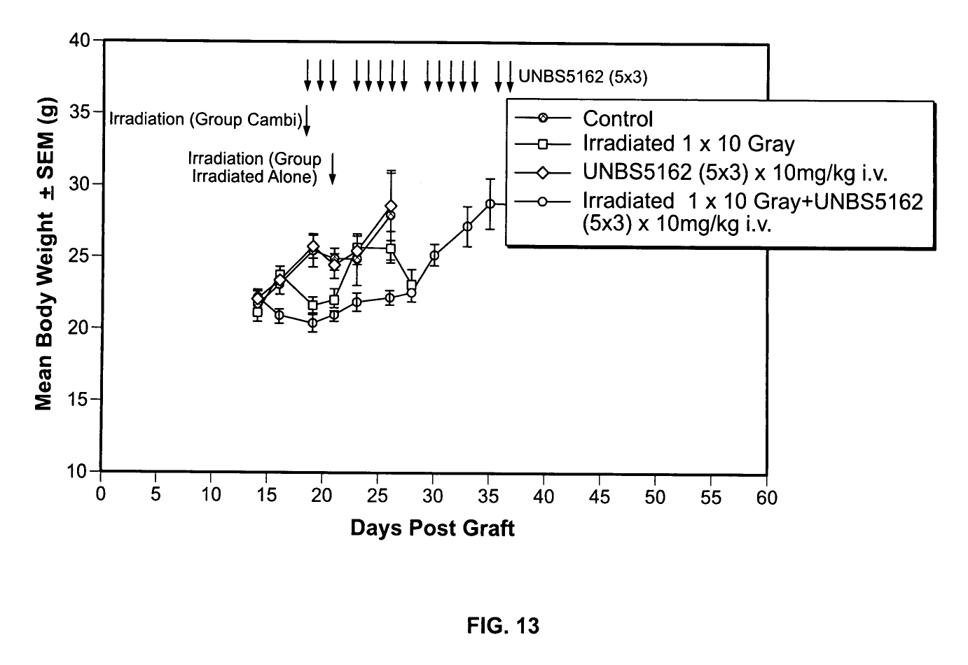

The experiment comprised 4 groups of II mice, one group with mice receiving the vehicle only (Ct group; black dots) and 3 groups receiving the treatments as follows: UNBS5162 alone dosed i.v. at 10 mg/kg (in lactic acid vehicle), one times a week for three weeks (squares); Temodal (Temodal 250 mg, Schering Plough, Belgium) dosed alone p.o. at 40 mg/kg three times a week for three weeks (rhombs) and Temodal administered p.o. at 40 mg/kg three times a week for three weeks followed by UNBS5162 injected i.v. at 10 mg/kg once a week for three weeks (open circles). As shown on FIG. 13, the treatment comprising a combination of Temodal followed by UNBS5162 significantly prolonged the survival period of tumor bearing mice. Thus, combining pro-autophagic therapy with an inhibitor of pro-angiogenic chemokines might represent a new manner to treat glioblastoma, at least some types that express high levels of pro-angiogenic chemokines.

### UNBS5162 as Potential Radiosensitizer in Rat Gliosarcoma Model: Ongoing In Vivo Study

The purpose of the present study is to evaluate the in vivo efficacy of treatment withUNBS5162 after gamma-knife irradiation on the rat 9 L gliosarcoma orthotopic tumor model.

The treatment protocol includes following conditions:

Gamma-Knife (Elekta Instrument, Sweden):
Dose: 70 Gy (GRAY)
1 irradiation D10 post-graft (˜20 minutes)
Irradiation source: cobalt

UNBS5162:
Vehicle: 1.35 eq lactic acid (L6661-500 mL, Sigma-Aldrich).
Route: i.p.
Dose (mg/kg): 10 mg/kg (1.35 eq lactic acid)
Dose volume: 1 ml/rat
Schedule: 5 injections/week for 3 weeks (5i×3w)

Groups:

Control

Control=>CTRL

Gamma-Knife=>group I

UNBS5162 (5i×3w)×10 mg/kg i.p.=>group 2
Gamma-Knife+UNBS5162 (5i×3w)×10 mg/kg i.p.=>group 3
   All treatments start on Day 10 post-tumor graft.

Parameters to be Monitored:

Body weight (3 times per week)

Survival (Kaplan-Meier)

Table of sacrifice or death

Data Analysis:

Statistical comparisons between the control group and the treated groups are performed with the non-parametric Mann-Whitney U-test. Survival analysis is done either by calculating the % T/C-value or either with Kaplan-Meier analysis and Log-rank statistics. All statistical analyses are carried out using Statistica software (Statsoft, Tulsa, Okla.). The cut-off level for statistical significance is set at a p-value of p<0.05.

### Histology:

Tumor (brains) are carefully isolated, processed in paraffin blocs and stored before histological analysis.

Comparable findings as found in the above examples are expected.

### Example 17

UNBS5162 N-{2-[2-(dimethylamino)ethyl]-1,3-dioxo-2,3-dihydro-1 H-benzo[de]isoquinolin-5-yl}urea enhances Taxol-mediated anti-tumor activity when administered at the same time or after Taxol treatment —NSCLC model.

The purpose of the present study was to evaluate the in vivo efficacy of UNBS5162 on human A549 NSCLC orthotopic nude mice model. The endpoint in this experiment was the survival period of the tumor-bearing mice following treatment, as indicated.

The experiment comprised 6 groups of II mice, one group with mice receiving the vehicle only (Ct group) and 5 groups receiving the treatments as indicated in the Table 13. UNBS5162 was dosed i.v. at 10 mg/kg (in lactic acid vehicle). Taxol (Paclitaxel 6 mg/mL, Bristol-Myers Squibb) was dosed i.v. at 20 mg/kg.

TABLE 13
Summary of the experiment

| | | Administration | | |
|---|---|---|---|---|
| Compound | Route | schedule | Dose (mg/kg) | T/C (%) |
| UNBS5162 | i.v. | (3i × 6 w) (a) | 10 (a) | 102 |
| Taxol | i.v. | (1i × 3 w) (b) | 20 (b) | 128 |
| UNBS5162 + Taxol | i.v. | (a)D14 + (b)D14 | 10 (a) + 20 (b) | 136 |
| (combination I) | | | | |
| UNBS5162 + Taxol | i.v. | (a)D14 + (b)D56 | 10 (a) + 20 (b) | 84 |
| (combination 2) | | | | |
| Taxol + UNBS5162 | i.v. | (b)D14 + (a)D35 | 10 (a) + 20 (b) | 150 |
| (combination 3) | | | | |

Table 13 presents the treatment schedule and the results in terms of T/C values. The T/C-values were calculated by dividing the day of sacrifice of the median mouse in a treated group T by the day of sacrifice of the median mouse in the control group C, the latter said to be 100%. A T/C value of 130% or higher is considered to be relevant. In contrast, a T/C-value of 75% or less is considered to indicate toxicity of treatment.

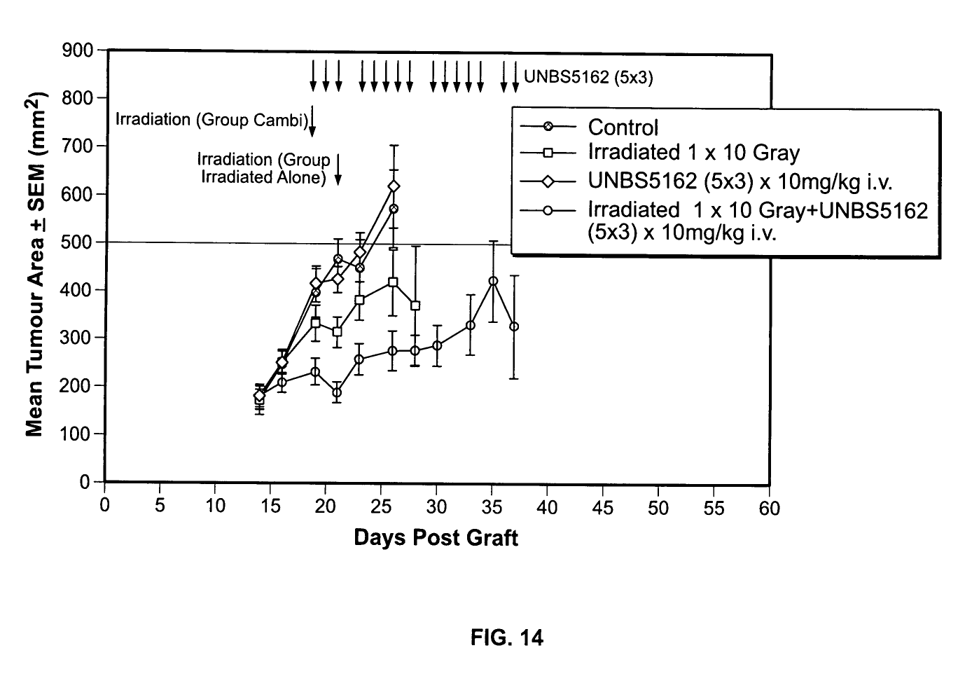

A prolongation of survival of tumor-bearing mice was observed with UNBS5162 in combination with Taxol. Combination 1 (UNBS5162 D14+Taxol D14, i.e. both compounds administered at the same time starting 14 days post-tumor graft): TIC=136% and combination 3 (Taxol D14+UNBS5162 D35; i.e. treatment with UNBS5162 started after the Taxol treatment): TIC=150%. The results are depicted in FIG. 14

Conclusion:

Results obtained in this study show that UNBS5162 (10 mg/kg i.v.) in combination with Taxol (20 mg/kg i.v.) significantly increased the survival of A549 NSCLC. Moreover, no relevant changes in body weights were observed after treatment with UNBS5162 or Taxol or both compounds in combination, indicating that treatment was well tolerated by the mice orthotopic xenograft-bearing mice.

### Example 18

### UNBS5162 Acts as Radiosensitizer in Head and Neck Model

Ongoing In Vivo Study:

### Aim:

The purpose of the present study is to evaluate the in vivo efficacy of treatment with UNBS5162 after tumor irradiation on the syngeneic SCVII head and neck tumor after orthotopic inoculation into conventional mice.

### Project:

Analyse the effect of combining radiotherapy with UNBS5162.

The experiment comprises 6 groups of II mice. The treatments will start at Day 5 post-tumor graft.

Group I: Control; mice receiving the vehicle only.
Group 2: UNBS5162; mice receiving UNBS5162 dosed i.v. at 10 mg/kg (in lactic acid vehicle); 5 injections per week, three weeks of treatment (5×3).
Group 3: Radiotherapy 5 Gray; one single irradiation on Day 5 post-tumor graft. Radiotherapy will be done at Bordet Instut, Brussels, Belgium. Irradiation will be focalised in the tumor centre and to avoid to irradiate healthy tissue.
Group 4: Radiotherapy 10 Gray; one single irradiation on Day 5 post-tumor graft.
Group 5: Combination UNBS5162 and radiotherapy 5 Gray; one single irradiation on Day 5 post-tumor graft with UNBS5162 dosed i.v. at 10 mg/kg (in lactic acid vehicle); 5 injections per week, three weeks of treatment (5×3) with first injection on Day 5 post-tumor graft (just before the radiotherapy).
Group 6: Combination UNBS5162 and Radiotherapy 10 Gray; one single irradiation on Day 5 post-tumor graft with UNBS5162 dosed i.v. at 10 mg/kg (in lactic acid vehicle); 5 injections per week, three weeks of treatment (5×3) with first injection on Day 5 post-tumor graft ((just before the radiotherapy). The endpoint in this experiment is the survival period of the tumor-bearing mice following the treatments, as indicated above. Survival will be analyzed by means of Kaplan-Meier curves.

### Rationale:

Our data obtained on the syngeneic MXT-HI mammary tumor after subcutaneous inoculation into conventional mice revealed that irradiation (1×10 gray) combined with UNBS5162 treatment (10 mg/kg i.v.; 5i×3w) significantly increased the survival of subcutaneous MXT-HI-bearing mice (Kaplan-Meier (Log-rank statistics) analysis: p=0.001).

Tamatani et al. (Int J Oncol 31, 2007; Int J Cancer. 108, 2004) demonstrated that that the combination of radiotherapy and cepharanthin (a biscoclaurine alkaloid extracted from the roots of Stephania cepharantha hayata reported to be able to inhibit NF-kappa B activation and IL-6 and IL-8 (CXCL8) production) could enhance radiosensitivity in the treatment of human oral cancer.

Our data obtained on prostate cancer models revealed UNBS5162 as potent inhibitor of pro-angiogenic CXC chemokines, among which IL-8 (CXCL8).

Recent reports demonstrating chemokine involvement in radioresistance and their up-regulation by radiotherapy (Bastianutto et al. Cancer Res. 67, 2007; Klopp et al., Cancer Res. 67, 2007).

Comparable findings as found in the above examples are expected.

### Example 19

### UNBS5162 in the Treatment of Breast Cancer

The original MXT mammary tumor model is a transplantable subcutaneous model initially developed by Watson et al. (Cancer Research 37, 3344-3348, 1977). Briefly, the tumors were induced in female BD2F mice carrying a pituitary isograft under the kidney capsule between 4 and 16 weeks of age. Urethan (dissolved in distilled water) was injected i.p. 10 weeks between 6 and 15 weeks of age. Tumors appeared between 12 and 15 months of host age and were serially transplanted with the use of a trocar to implant pieces s.c. into syngeneic mice. The HI-MXT variant of mammary carcinoma model used here is a hormone-insensitive (HI) form of the MXT model (Kiss et al., Cancer Research 49, 2945-2951, 1989). The MXT-HI models used in our laboratory involve subcutaneously injecting MXT-HI tumor fragments into the flanks of B6D2F1 mice. Without treatment, the mice die between the 25th and 28th day following injection with MXT-HI tumor fragments.

The purpose of the present study was to evaluate the in vivo efficacy of treatment with UNBS5162 after tumor irradiation on the syngeneic MXT-HI mammary tumor after subcutaneously inoculation into conventional mice.

### Materials, Reagents and Equipment Used

Syringe Terumo with needle 1 ml VWR, Leuven, Belgium)
Saline solution (Baxter, Brussels, Belgium)
Trocar (13-gauge)
Petri dishes (Nunc, VWR, Leuven, Belgium)
Scalpel
Scissor
Electronic balance (Sartorius)
Electronic calliper (DIGIT-CAL, Capa system)
SLS Philips simulator
Linear accelerator (SL75, Elekta, Crawley, UK)

Body weight and tumor size measurements were automatically filed with the software PAC2000 (Viewpoint 1999).

### Origin of Cancer Fragments Used for Inoculation

The original MXT mammary tumor model is a transplantable subcutaneous model initially developed by Watson et al. (Cancer Research 37, 3344-3348, 1977). The tumors were induced in female BD2F mice carrying a pituitary isograft under the kidney capsule between 4 and 16 weeks of age. Urethan (dissolved in distilled water) was injected i.p. 10 weeks between 6 and 15 weeks of age. Tumors appeared between 12 and 15 months of host age and were serially transplanted with the use of a trocar to implant pieces s.c. into syngeneic mice. The HI-MXT variant of mammary carcinoma model used here is a hormone-insensitive (HI) form of the MXT model (Kiss et al., Cancer Research 49, 2945-2951, 1989). The MXT-HI models used in our laboratory involve subcutaneously injecting MXT-HI tumor fragments into the flanks of B6D2F1 mice. Without treatment, the mice die between the 25th and 28th day following injection with MXT-HI tumor fragments.

Cancer cells were grafted in animals to create “bank animals”. The transfer from one passage to the next is performed between day 20 and day 25. At each transfer, MXT-HI tumor is minced into 1-mm³ pieces, and these pieces are randomly s.c. inoculated into the right flanks of “new bank” mice by means of a trocar (13-gauge).

### Animals

Healthy 5-week-old female B6D2F1 mice (−20 g on arrival) were supplied by Charles River Laboratories (Brussels, Belgium) at least one week prior to inoculation. All the animals were housed in plastic cages in a room with controlled temperature (22±2° C.), light exposure (from 7:00 am to 7:00 pm), and 55±5% relative humidity. Animals had free access to laboratory chow and water. Animals were handled and maintained in accordance with Authorization No. LA 1230509 of the Animal Ethics Committee of the Federal Department of Health, Nutritional Safety and the Environment (Belgium).

Mice were twice daily observed for general health status and sacrificed (by Nembutal injection or by CO₂) according to the criteria of the Ethical Committee:

Body weight loss of more than 25%

Decreased mobility

Hunched back

Abnormal breathing

Convulsions

Ulcerating tumor

Bad general health status

This day of sacrifice is said to be the day of death of the mice.

### Inoculation

After the adaptation period, animals were inoculated subcutaneously with MXT-HI tumor. Briefly, “bank animals” were sacrificed and MXT-HI tumors were isolated from the animals, kept in saline solution and minced into 1 mm³ pieces. These pieces (one/animal) were randomly s.c. inoculated into the right flanks of mice by means of a trocar.

### Compound

UNBS5162 (batch 18)

### Formulations

UNBS5162: 1.35 equivalents of lactic acid in 0.9% NaCl, pH 5.75 (1 mg/mL; solution)

### Treatment Schedule

UNBS5162 was dosed intravenously in accordance with UNBS procedure #002. The dosing volume was fixed at 200 μL/mouse.
Group 1: Control (lactic acid vehicle) (51/week×3 weeks)
Group 2: UNBS5162 (10 mg/kg, 51/week×3 weeks)
Group 3: Irradiated (10 gray, 1 irradiation)
Group 4: Irradiated (10 gray, 1 irradiation)+UNBS5162 (10 mg/kg, 51/week×3 weeks)
For group 4, the first administration of UNBS5162 was performed at the morning of the day of irradiation (5 Dec. 2007).

### Irradiation

Briefly, animals were anaesthetized, using a syringe, with 150 μl of Rompun/Ketalar mixture (1 part Rompun/1 part Ketalar/4.5 parts saline) injected intraperitoneally. After approximately 5 minutes and after verification that animals were completely asleep, an irradiation simulation was performed first to focalise irradiation in the tumor centre and to avoid to irradiate healthy tissue. Simulation was performed individually on each animal to adapt irradiation to tumor size. In a second time, the tumor was irradiated with a dose of 10 gray (5 MV photons from a linear accelerator at a source skin distance of 1 m).

The irradiation was performed by Dr. Boterberg (department of Radiotherapy UZ Gent).

For technical reasons, irradiation was not performed the same day for all animals. Tumor irradiation was performed on day 14 post-graft for group 4 (irradiated+UNBS5162) and on day 16 post-graft for group 3 (irradiated alone).

Experimental Parameters

Body weights were recorded 3 times weekly on Monday, Wednesday and Friday as the first indication of potential side effects.

Tumor growths were recorded 3 times weekly on Monday, Wednesday and Friday.

Day of death.

### Data Analysis

Statistical comparisons between the control group and the treated groups were performed with the non-parametric Mann-Whitney U-test. Survival analysis was done either by calculating the % T/C-value or with Kaplan-Meier analysis and Log-rank statistics. All statistical analyses were carried out using Statistica software (Statsoft, Tulsa, Okla.). The cut-off level for statistical significance was set at a p-value of p<00.05.

The study (except irradiation) was performed by Fabrice Ribaucour and Gwenael Dielie who were responsible for experimental study conduct and data analysis, first data audit was performed by Ellen Van Der Aar, final data audit was performed by Robert Kiss and report writing was performed by Mischael Dehoux.

### Results

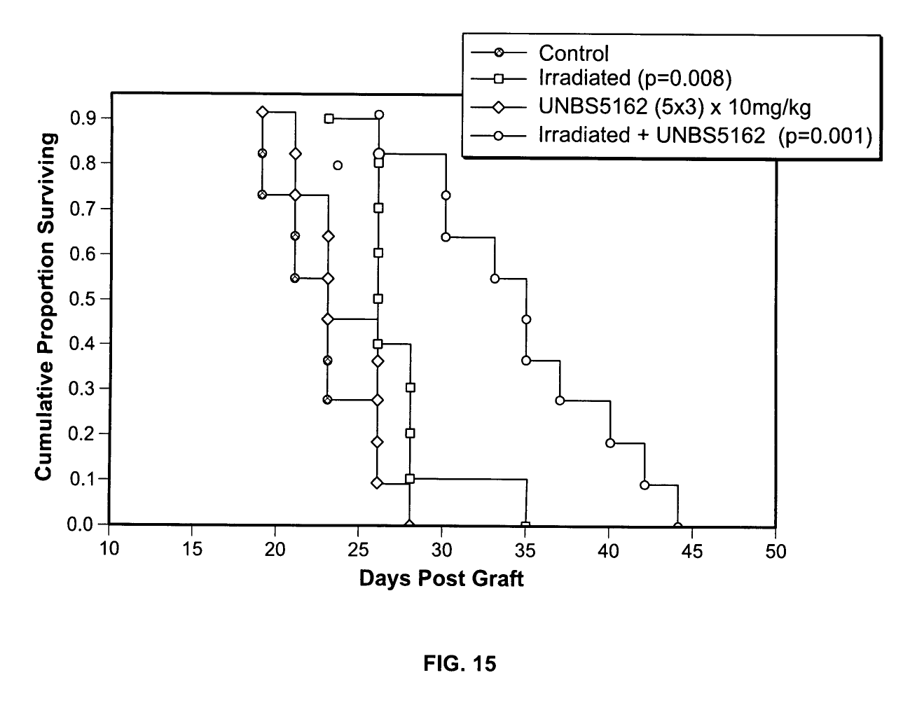

FIG. 15 shows the evolution of body weight in function of time and treatment. No significant changes in body weights were observed in groups 2 (UNBS5162 alone) and 3 (irradiation alone) indicating that treatment was well tolerated by the mice (compared to control group 1). A significant decrease in body weight was observed in group 4 (UNBS5162 and irradiation) compared to control. This, however, could be explained by the strong increase in body weight in the control group, which is likely due to the important development of tumor mass.

The related statistical analysis is shown in Table 14 and the mean (±SEM) of the body weights is presented in Table 15.

TABLE 14
P-values after statistical analysis (compared to Ctrl group)
of body weight changes versus time for female B6D2F1 mice
grafted subcutaneously with MXH-HI mammary tumor fragments
left untreated (control) or treated i.v. (5i × 3 w)
with UNBS5162 at a targeted dose level of 10 mg/kg and/or
irradiated (1 irradiation) at a targeted dose level of 10
gray (n.s. = not significant).

| | Irradiation 10 | UNBS5162 | Irradiation + |
|---|---|---|---|
| Days | gray | 10 mg/kg | UNBS5162 |
| 14 | n.s. | n.s. | n.s. |
| 16 | n.s. | n.s. | 0.02 |
| 19 | 0.01 | n.s. | 0.003 |
| 21 | n.s. | n.s. | 0.0007 |
| 23 | n.s. | n.s. | n.s. |
| 26 | n.s. | n.s. | 0.02 |

TABLE 15
Mean body weight changes versus time for female B6D2F1 mice grafted
subcutaneously with MXH-HI mammary tumor fragments left untreated (control) or
treated i.v. (5i × 3w) with UNBS5162 at a targeted dose level of 10 mg/kg and/or
irradiated (1 irradiation) at a targeted dose level of 10 gray.

| | | Day | | | | | | | | | | | |
|---|---|---|---|---|---|---|---|---|---|---|---|---|---|
| | | 14 | 16 | 19 | 21 | 23 | 26 | 28 | 30 | 33 | 35 | 37 | 40 |
| Control | Weight (g) | 21.65 | 23.05 | 25.45 | 24.84 | 24.80 | 27.93 | | | | | | |
| | SEM | 0.49 | 0.66 | 1.19 | 0.81 | 1.82 | 3.11 | | | | | | |
| Irradiation 10 | Weight (g) | 21.16 | 23.70 | 21.59 | 22.03 | 25.64 | 25.67 | 23.05 | | | | | |
| gray | SEM | 0.61 | 0.63 | 0.59 | 0.74 | 1.0.6 | 1.0.9 | 1.14 | | | | | |
| UNBS5162 | Weight (g) | 22.13 | 23.39 | 25.72 | 24.35 | 25.46 | 28.52 | | | | | | |
| (5i × 3w) × 10 Mg/kg | SEM | 0.49 | 0.53 | 0.81 | 0.87 | 1.00 | 2.33 | | | | | | |
| Irradiation 10 | Weight (g) | 22.18 | 20.89 | 20.44 | 20.97 | 21.88 | 22.18 | 22.51 | 25.17 | 27.18 | 28.75 | 28.68 | 23.95 |
| gray + | SEM | 0.49 | 0.46 | 0.61 | 0.46 | 0.62 | 0.49 | 0.62 | 0.77 | 1.40 | 1.70 | 1.97 | 1.85 |
| UNBS5162 | | | | | | | | | | | | | |
| (5i × 3w) × 10 Mg/kg | | | | | | | | | | | | | |

FIG. 16 shows the evolution of the tumor sizes in function of time and treatment. As can be seen, a significant decrease in tumor area was observed with irradiation combined with UNBS5162 treatment. In contrast, no relevant effects were observed with irradiation alone or UNBS5162 alone.

The related statistical analysis is shown in Table 16 and the mean (±SEM) of the tumor sizes is presented in Table 17.

TABLE 16
P-values after statistical analysis (compared to Ctrl group) of tumor
size evolution versus time for female 86D2F1 mice grafted
subcutaneously with MXH-HI mammary tumor fragments left untreated
(control) or treated i.v. (5i × 3 w) with UNBS5162 at a targeted
dose level of 10 mg/kg and/or irradiated (1 irradiation) at a targeted
dose level of 10 gray (n.s. = not significant).

| | Irradiation 10 | UNBS5162 | Irradiation + |
|---|---|---|---|
| Days | gray | 10 mg/kg | UNBS5162 |
| 14 | n.s. | n.s. | n.s. |
| 16 | n.s. | n.s. | n.s. |
| 19 | n.s. | n.s. | 0.01 |
| 21 | 0.02 | n.s. | 0.0005 |
| 23 | n.s. | n.s. | 0.03 |
| 26 | n.s. | n.s. | 0.03 |

TABLE 17
Mean tumor sizes (mm²) evolution versus time for female B6D2F1 mice grafted
subcutaneously with MXH-HI mammary tumor fragments left untreated (control) or treated
i.v. (5i × 3w) with UNBS5162 at a targeted dose level of 10 mg/kg and/or irradiated
(1 irradiation) at a targeted dose level of 10 gray.

| | | Day | | | | | | | | | | | |
|---|---|---|---|---|---|---|---|---|---|---|---|---|---|
| | | 14 | 16 | 19 | 21 | 23 | 26 | 28 | 30 | 33 | 35 | 37 | 40 |
| Control | Tumour | 169.43 | 245.39 | 395.42 | 466.25 | 448.77 | 572.32 | | | | | | |
| | size | | | | | | | | | | | | |
| | SEM | 30.34 | 32.21 | 50.86 | 43.53 | 60.62 | 79.94 | | | | | | |
| Irradiation | Tumour | 170.88 | 250.65 | 331.81 | 314.41 | 378.26 | 417.71 | 369.45 | | | | | |
| 10 gray | size | | | | | | | | | | | | |
| | SEM | 21.29 | 24.84 | 37.70 | 31.52 | 40.04 | 70.88 | 126.36 | | | | | |
| UNBS5162 | Tumour | 178.52 | 250.08 | 414.11 | 423.71 | 481.56 | 619.21 | | | | | | |
| (5i × 3w) | size (mm²) | | | | | | | | | | | | |
| Mg/kg | SEM | 21.42 | 24.19 | 38.67 | 27.91 | 42.33 | 85.37 | | | | | | |
| Irradiation | Tumour | 179.94 | 207.42 | 230.15 | 187.53 | 256.66 | 274.61 | 276.00 | 286.84 | 329.50 | 422.00 | 326.53 | 225.37 |
| 10 gray | size | | | | | | | | | | | | |
| UNBS5162 | SEM | 23.62 | 21.00 | 29.23 | 21.79 | 33.07 | 41.34 | 31.33 | 42.00 | 62.70 | 85.64 | 10870 | 207.81 |
| (5i × | | | | | | | | | | | | | |
| Mg/kg | | | | | | | | | | | | | |

Table 18 shows the day of death or sacrifice of the individual mice, whereas the TIC-values (expressed in %) are summarized below. The T/C-values were calculated by dividing the day of death or sacrifice of the median mouse in a treated group T by the day of death or sacrifice of the median mouse in the control group C, the latter said to be 100%. A TIC-value of 130% or higher is considered to be relevant. In contrast, a T/C-value of 75% or less is considered to indicate toxicity of treatment.

TABLE 18
Table of deaths: Number of female B6D2F1 mice grafted subcutaneously
with MXH-HI mammary tumor fragments left untreated (control) or treated
i.v. (5i × 3w) with UNBS5162 at a targeted dose level of 10 mg/kg
and/or irradiated (1 irradiation) at a targeted dose level of 10
gray. Mice were sacrificed according to the criteria of the Ethical
Committee (tumor size >500 mm2) or when moribund.

| DRUGS | | | DAYS POST GRAFT | | | | | | | | | | | | |
|---|---|---|---|---|---|---|---|---|---|---|---|---|---|---|---|
| | | Scheduled | | | | | | | | | | | | | T/C |
| Name | Route | treatment | 19 | 21 | 23 | 26 | 28 | 30 | 33 | 35 | 37 | 40 | 44 | | %) |
| Control | i.v. | (5 × 3) | 3 | 2 | 3 | 2 | 1 | / | | | | | | | |
| Irradiated | / | 1 × 10 | | | 1 | 5 | 3 | | | 1 | / | | | | 113 |
| | | gray | | | | | | | | | | | | | |
| UNBS5162 | i.v. | (5 × 3) × 10 mg/kg | 1 | 2 | 3 | 4 | 1 | / | | | | | | | 100 |
| Irradiated + | /+I.v. | 1 × 10 gray + | | | | 2 | | 2 | 1 | 2 | 1 | 2 | 1 | / | 152 |
| UNBS5162 | | (5 × 3) × 10 | | | | | | | | | | | | | |

Rem: Experiments were performed on 11 mice per group, except for the irradiated group, where n = 10.

A prolongation of survival was observed after irradiation combined with UNBS5162 treatment: T/C=152%.
No effects upon survival were observed after treatment with UNBS5162 alone (T/C=100%) or irradiation alone (T/C=113%).

FIG. 16 shows the Kaplan-Meier graph. The prolongation of survival after irradiation and treatment with UNBS5162 as evidenced by the % T/C-values, was confirmed with the Kaplan-Meier (Log-rank statistics) analysis (respectively p=0.001). Although irradiation alone (10 gray) was not considered as significant based on T/C values (T/C=113%), the Kaplan-Meier (Log-rank statistics) analysis shows a significant effect of irradiation alone on survival (p=0.008). No prolongation of survival was observed with UNBS5162 alone (compared to Control).

### Discussion

Naphthalimides, a class of compounds which bind to DNA by intercalation have shown high anticancer activity against a variety of murine and human tumor cells. In the present study, it has been demonstrated that compound UNBS5162, a novel proprietary naphthalimide derivative, is a potential novel anti-cancer compound.

Results obtained in this study show that irradiation (1×10 gray) combined with UNBS5162 treatment (10 mg/kg i.v.; 5i×3w) significantly increased the survival of subcutaneous MXT-HI-bearing mice.

A significant decrease in body weight was observed compared to the control group. This could be explained by the strong increase in body weight in the control group, which is likely due to the important development of tumor mass.

### Conclusion:

Results obtained in this study allow concluding that compound UNBS5162 when combined with irradiation is effective in the MXT-HI mammary cancer model. A clear-cut synergistic effect in temms of therapeutic benefits is observed when treating MXT-HI tumor-bearing mice with UNBS5162 after irradiation.

Many other variations of the present invention will be apparent to those skilled in the art and are meant to be within the scope of the claims appended hereto, including but not limited to treatment of particular proliferative diseases utilizing the compositions and methods of treatment recited herein as well as other numerical parameters described in the examples, and any combination thereof.

The disclosures of all of the publications recited herein are hereby incorporated by reference in their entireties.

1. A method of treating esophageal cancer comprising administering to a patient in need thereof, N-{2-[2-(dimethylamino)ethyl]-1,3-dioxo-2,3-dihydro-1H-benzo[de]isoquinolin-5-yl}urea or a pharmaceutically acceptable salt thereof and/or a metabolite thereof in an amount effective to down-regulate one or more esophageal cancer cell pro-angiogenic chemokines.

2. The method of claim 1 wherein the chemokine is selected from the group consisting of CXCL-1, CXCL-2, CXCL-8 and combinations thereof.

3. The method of claim 1 further comprising administering an antineoplastic agent.

4. The method of claim 3 wherein the antineoplastic agent is selected from the group consisting of taxol, temodal, dacarbazine, and pharmaceutically acceptable salts thereof and/or metabolites thereof.

5. The method of claim 3 wherein the antineoplastic agent is cisplatin.

6. The method of claim 5 wherein the esophageal tumor growth is slowed.

7. The method of claim 5 wherein the esophageal tumor growth is stopped.

8. The method of claim 5 wherein the esophageal tumor size is decreased.

9. The method of claim 5 wherein the patient experiences less hematotoxicity compared to treatment with a therapeutically equivalent amount of amonafide.

10. The method of claim 3 wherein the antineoplastic agent is pro-autophagic.

11. The method of claim 3 wherein the antineoplastic agent is pro-apoptotic.

12. The method of claim 1 wherein the effective amount is about at least about 10 mg/kg.

13. The method of claim 1 wherein one or more courses of treatment comprises administering one daily dose for at least about 5 consecutive days.

14. The method of claim 1 wherein one or more courses of treatment comprises administering one daily dose for at least about 3 times a week for a duration selected from the group consisting of (i) at least about 3 weeks, (ii) at least about 5 weeks and (iii) at least about 9 weeks.

15. The method of claim 5 wherein the dose of cisplatin is at least about 5 mg/kg.

16. The method of claim 15 wherein one or more courses of treatment comprises administering one daily dose per week for 5 weeks.

17. The method of claim 3 wherein the antineoplastic agent is temodal, or pharmaceutically acceptable salts thereof and/or metabolites thereof administered at a dose of at least about 80 mg/kg at least about 3 injections per week for about 3 weeks.

18. The method of claim 3 wherein the antineoplastic agent is temodal, or pharmaceutically acceptable salts thereof and/or metabolites thereof administered at a dose of 80 mg/kg at 3 injections per week for 9 weeks.

19. The method of claim 3 wherein the antineoplastic agent is dacarbazine, or pharmaceutically acceptable salts thereof and/or metabolites thereof administered at a dose of about 80 mg/kg at 3 injections per week for 3 weeks.

20. The method of claim 3 wherein the antineoplastic agent is dacarbazine, or pharmaceutically acceptable salts thereof and/or metabolites thereof administered at a dose of 80 mg/kg at 3 injections per week for 9 weeks.

21. The method of claim 1 wherein the antineoplastic agent is dacarbazine, or pharmaceutically acceptable salts thereof and/or metabolites thereof administered at a dose of about 10 mg/kg at 3 injections per week for 3 weeks.

22. The method of claim 3 wherein the antineoplastic agent is dacarbazine, or pharmaceutically acceptable salts thereof and/or metabolites thereof administered at a dose of 10 mg/kg at 3 injections per week for 9 weeks.

23. A method of treating esophageal cancer comprising administering to a patient in need thereof, a substituted naphthalimide derivative represented by the structural formula (I) 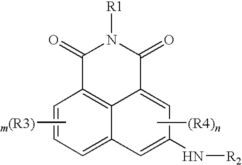
wherein:
R₁ is mono- or diC₁₋₄ alkylamino-C₁₋₄ alkyl;
each of R₃ and R₄ is independently selected from the group consisting of hydrogen, halogen, C₁₋₄ alkyl, C₁₋₄ alkoxy, C₁₋₄ alkylthio, nitro, cyano, amino, protected amino and halo C₁₋₄ alkyl;
m is the number of substituents R₃ and ranges from 0 to 3;
n is the number of substituents R₄ and ranges from 0 to 2; and
R₂ is CONH₂
and/or a pharmaceutically acceptable salt thereof and/or a solvate thereof and/or a metabolite thereof in an amount effective to down-regulate one or more esophageal cancer cell pro-angiogenic chemokines.

24. A pharmaceutical composition for injection comprising a therapeutically effective amount of N-{2-[2-(dimethylamino)ethyl]-1,3-dioxo-2,3-dihydro-1H-benzo[de]isoquinolin-5-yl}urea for the treatment of esophageal cancer in a pharmaceutically acceptable carrier comprising a liquid comprising an amount of lactic acid suitable for parenteral administration.
